(19) United States
(12) Patent Application Publication
(10) Pub. No.: US 2024/0294604 A1
(43) Pub. Date: Sep. 5, 2024

(54) sBCMA Variants and FC Fusion Proteins Thereof

(71) Applicant: The Board of Trustees of the Leland Stanford Junior University, Stanford, CA (US)

(72) Inventors: Amato J. Giaccia, Stanford, CA (US); Yu Miao, Stanford, CA (US); Kaushik Thakkar, Stanford, CA (US); Kazue Mizuno, Stanford, CA (US)

(73) Assignee: The Board of Trustees of the Leland Stanford Junior University, Stanford, CA (US)

(21) Appl. No.: 18/041,527

(22) PCT Filed: Aug. 11, 2021
(86) PCT No.: PCT/US2021/045615

§ 371 Date: Feb. 13, 2023

Related U.S. Application Data
(60) Provisional application No. 63/064,880, filed on Aug. 12, 2020.

(51) Int. Cl. C07K 14/705; A61K 38/00; A61P 35/00

(52) U.S. Cl. CPC C07K 14/70578; A61P 35/00; A61K 38/00; C07K 2319/30

(57) ABSTRACT

The invention is directed to novel sBCMA variants and sBCMA variant—Fc fusion proteins, polynucleotides encoding the sBCMA variants and/or sBCMA variant—Fc fusion proteins, methods of making the sBCMA variants and/or sBCMA variant—Fc fusion proteins, and methods of using compositions comprising the sBCMA variants and/or sBCMA variant—Fc fusion proteins, for example, in treating diseases such as tumors/cancers, immunoregulatory disorders, etc.

### SEQUENCE LISTING

The instant application contains a Sequence Listing which has been submitted electronically in ASCII format and is hereby incorporated by reference in its entirety. Said ASCII copy, created on Aug. 11, 2020, is named 121076-5004-PR_ST25.txt and is 60.0 kilobytes in size.

### I. FIELD OF THE INVENTION

This invention relates to soluble B-cell maturation antigen (sBCMA) variants and sBCMA variant—Fc fusion proteins, polynucleotides encoding the sBCMA variants and/or sBCMA variant—Fc fusion proteins, methods of making the sBCMA variants and/or sBCMA variant—Fc fusion proteins, and methods of using compositions comprising the sBCMA variants and/or sBCMA variant—Fc fusion proteins, for example, in treating diseases such as tumors/cancers, immunoregulatory disorders, fibrosis, etc.

### II. BACKGROUND OF THE INVENTION

The Tumor Necrosis Factor (“TNF”) family consists of pairs of ligands and their specific receptors referred to as TNF family ligands and TNF family receptors (Bazzoni et al. N. Engl. J. Med. 1996, 334(26):1717). The family is involved in the regulation of the immune system and possibly other non-immunological systems. The regulation of TNF family signaling can result in a large number of subsequent events. TNF can initiate the general protective inflammatory response of an organism to foreign invasion that involves the altered display of adhesion molecules involved in cell trafficking, chemokine production to drive specific cells into specific compartments, and the priming of various effector cells. As such, the regulation of these pathways has clinical potential (U.S. Pat. No. 9,650,430 B2).

B-cell maturation antigen (BCMA) is a member of the tumor necrosis factor receptor superfamily member. The amino acid sequence of the extracellular domain of BCMA is shown in FIG. 7. For example, BCMA is a receptor for A Proliferation Inducing Ligand (APRIL) and j-cell Activating Factor of the TNF family (BAFF). Anti-BCMA antibodies, including an antibody drug conjugate (ADC), have shown initial success in treating cancer in early testing, as have BCMA bispecific T cell engaging antibodies and CAR-T constructs using BCMA.

APRIL is previously described in WO 99 12965 and U.S. Pat. No. 7,276,241 B2, which are incorporated by reference herein. The amino acid sequence of the extracellular domain of APRIL is shown in FIG. 7. APRIL expression and functional studies suggest that this protein is utilized by tumor cells to induce rapid proliferation. In addition, APRIL may act in other disease settings, for example, in cell proliferative diseases, such as those that occur in connection with some autoimmune diseases (e.g., lupus) or in inflammatory diseases where cell populations expand rapidly (e.g. bacterial sepsis) (U.S. Pat. No. 7,276,241B2).

BAFF is previously described in WO/0012964 and U.S. Pat. No. 9,650,430 B2, which are incorporated by reference herein. The amino acid sequence of the extracellular domain of BAFF is shown in FIG. 7. BAFF is a cell survival and maturation factor for B cells, and overproduction of BAFF is associated with systemic autoimmune disease. In humans, high levels of BAFF are detectable in the blood of a proportion of patients with autoimmune rheumatic diseases, particularly systemic lupus erythematosus and Sjögren's syndrome (Groom et al. J. Clin. Invest., 2002, 109:59; Zhang et al. J. Immunol., 2001, 166:6; Cheema et al. Arthritis Rheum. 2001, 44:1313, which are all incorporated by reference herein). BAFF is also an effective costimulator for T cells, and this costimulation occurs entirely through BAFF-R (Ng et al. J. Immunol., 2004, 173:807, incorporated by reference herein).

Transmembrane activator and CAML interactor (TACI) also known as tumor necrosis factor receptor superfamily member 13B (TNFRSF13B) is a type III transmembrane protein. Several proteins (BAFF/BLys, APRIL, Syndecan-2) have been identified as TACI ligands. The interaction of TACI with its ligands induces activation of the transcription factors NFAT, AP1, and NF-κ B and plays a crucial role in humoral immunity by regulation of B cell proliferation and survival. TACI activation of B cells leads to their differentiation and maturation, including antibody isotype switch, and T cell-independent antibody production (Chinen et al. J. Allergy Clin Immunol. 2011, 127(6): 1579, incorporated by reference herein).

APRIL and BAFF can bind to receptors, such as BCMA, BAFF-receptor (BAFFR) and TACI, and thus neutralizing APRIL and/or BAFF can be used for treating the diseases, e.g. cancers, autoimmune diseases and fibrosis arising from altered signaling pathways through BCMA, BAFFR and/or TACI.

Current treatments for cancer, immunomodulatory and fibrotic disorders are inadequate for many disease types, due to poor efficacy, low impact on survivorship, toxicity that causes severe side effects, or combinations thereof. Therefore, there is a need to identify and develop additional methods for treating cancers and/or immunomodulatory disorders which can provide efficacy without inducing severe side effects. The present invention satisfies this and other needs.

It is an object of the present invention to provide sBCMA variants or sBCMA variant—Fc fusion proteins having improved properties (e.g. increased binding affinity for APRIL and/or BAFF, etc.) as well as methods of making and using such sBCMA variants and/or Fc fusion proteins thereof in treating patients with cancers and/or immunomodulatory disorders.

### III. BRIEF SUMMARY OF THE INVENTION

The present invention provides, inter alia, sBCMA variants and Fc fusion proteins thereof, polynucleotides encoding the sBCMA variants and/or Fc fusion proteins thereof, methods of making the sBCMA variants and/or Fc fusion proteins thereof, and methods of using the sBCMA variants and/or Fc fusion proteins thereof, particularly for treating diseases such as cancers or immunomodulatory disorders. In some embodiments, the sBCMA variants are used to treat cancers or immunomodulatory applications. In some embodiments, the sBCMA variant—Fc fusion proteins are used to treat cancers or immunomodulatory applications.

In one aspect, the invention provides compositions comprising a variant soluble B-cell maturation antigen (sBCMA) comprising at least one amino acid substitution as compared to SEQ ID NO:1, wherein said amino acid substitution is at a position number selected from the group consisting of 1, 2, 3, 4, 5, 6, 7, 9, 10, 11, 12, 14, 16, 19, 20, 22, 23, 25, 26, 29, 31, 32, 35, 36, 38, 39, 42, 43, 44, 45, 46, 47, 48, 49, 50, 51, 52, 53, and 54, wherein the numbering is according to the EU index.

In an additional aspect, the invention provides compositions as described herein, wherein said variant sBCMA has at least 80%, at least 85%, at least 90%, or at least 95% sequence identity to SEQ ID NO:1.

In a further aspect, the invention provides compositions as described herein, wherein said amino acid substitution(s) occur at one of said positions, two of said positions, three of said positions, four of said positions, five of said positions, six of said positions, seven of said positions, eight of said positions, or nine of said positions.

In an additional aspect, the invention provides compositions as described herein, wherein said amino acid substitution(s) is selected from the group consisting of M1A, M1C, M1I, M1R, M1T, M1V, L2C, L2S, Q3P, Q3R, M4E, M4I, M4T, M4V, A5T, G6E, Q7R, S9A, S9F, S9P, Q10H, Q10P, Q10R, N11D, N11S, E12K, F14L, S16G, S16N, S16R, H19L, H19Y, A20V, A20T, I22M, I22V, P23S, Q25R, L26F, S29A, N31D, N31S, T32A, T32I, T32P, L35S, L35P, T36A, T36I, T36P, Q38R, R39H, N42D, N42R, N42S, A43T, A43V, S44D, S44G, S44N, S44R, V45A, V45M, T46A, T46I, N47D, N47K, N47R, N47S, S48L, S48P, S48T, V49A, V49M, K50E, K50G, K50R, K50T, G51E, T52A, T52M, N53D, N53K, N53S, A54V, and A54T.

In a further aspect, the invention provides compositions as described herein, wherein said amino acid substitution(s) is selected from the group consisting of M1V, L2S, Q3P, M4T, S9P, N11D, S16G, H19Y, N31S, N31D, T32I, T36A, R39H, N47S, K50E, and N53E.

In an additional aspect, the invention provides compositions as described herein, wherein said amino acid substitution(s) is selected from the group consisting of S16G, H19Y and T36A.

In a further aspect, the invention provides compositions as described herein, wherein said amino acid substitutions are selected from the group consisting of L2S/S9P/E12K/N31D/T36A/N42S/N53S, M1V/T32P/T36A/T46I/N53D/A54V, Q3R/S16N/T36A/A43T, F14L/S16G/T36A/V45A/N47D, M1T/M4V/S9F/S16G/T32A/Q38R, M1A/S9A/Q38R, G6E/Q25R/Q38R, M1V/M4I/G6E/S9P/N11D/V49M/T52M/A54V, N11D/S16G/N31S, N11D/H9Y/I22M/T32P/N47S/N53S, G6E/Q7R/H19Y/L35S, H19Y/N42D/S48P/T52A, M1V/N31D/T32I/T36A, M1V/A5T/H19L/T36A, M1T/N31D/T32A/T36A/Q38R/S44D/V49A/K50E, M1V/T36A/Q38R/A43V, M1V/L2S/S9P/Q10H/T36A/Q38R/K50G, T36A/Q38R/N53S, M1T/L2S/L35P/T36A/Q38R/T46A/K50R, A5T/A20V/T36A/Q38R, M1T/S16G/I22V/T36A/S44G/T46A/V49A, S16G/T36A, M1I/N11D/S16G/I22M/S29A/T36A/S44G/K50R, M1C/L2C/Q3R/M4E/N11D/S16G/T36P, M1I/N11D/S16G/I22M/S29A/T36A/S44G/K50R, N11D/N31D/T32I/T36A/S44N/N47D/N53D, M1R/L2C/Q3R, H19Y/T36A/S44G, H19Y/T32I/T36A/V49A, H19Y/N31S/T36A/V45A, H19Y/N31S/T36A, H19Y/T36P/T52A, H19Y/N31D/T52M, M1V/H19Y/V45M, S16G/H19Y/N47D, S16G/H19Y/K50T, S16G/H19Y/S44N/K50R, N11D/H19Y/S48T, S9P/NI1D/S16R/T32A/Q38R/S44G/T46I/T52A/N53D/A54T, N11D/S16G/S44R, H19L/T32A/S44G/G51E/T52A, S16N/H19Y/T36A/K50R, M1V/H19Y/T36A/R39H/T46A, M1V/H19Y/T36A, H19Y/T36A/N42D/N47S/S48P, M1V/H19Y/T36A/S44G/N47D, M1V/H19Y/T36A/N42R/N53S, H19Y/L35P/T36A/N42D/T46I/V49A, Q3P/S9P/H19Y/N31S/T36A/R39H/N47R/K50E, M1V/H19Y/T36A/N42R/N53S, M1T/H19Y/T36A, M1V/S16N/H19Y/I22M/T36A, M1T/N11D/H19Y/T36A/N42S/V45A/N53S, N11D/S16G/H19Y/T36A/N47S/N53D, M1V/S9P/Q10P/S16G/H19Y/L26F/T36A/A43V/N53D, S16G/H19Y/T36A/V49A/N53D, S16G/T36A/A43T/S44G/V45M, M4V/S9P/S16G/T36A/Q38R, S9P/N11S/S16G/T36A/Q38R, N11D/E12K/S16R/T36A/T52M, M4V/T32I/T36A/Q38R/A43T/V45A/S48P, S9P/N11D/S16G/Q25R, M1T/A5T/S9P/S16G/Q25R/N31D/V49M, L2S/S9P/S16G/A20T/T32I/Q38R/N42D/T46A/S48L, S16G/Q25R/T46A, G6E/S9A/S16G/Q25R/N31D/N47S/T52M, H19Y/Q38R/T52M, N11D/H19Y/I22M/T32P/N47S/N53S, S16G/H19Y/T36A, S16G/H19Y/T36A/N53D, S9P/N11D/S16G/H19Y/T36A/N47S/N53D, Q3P/S9P/H19Y/N31S/T36A/R39H/N47R/K50E, M1V/L2S/M4T/N11D/H19Y/T36A, M1V/L2S/M4T/N11D/T36A, M1V/L2S/M4T/H19Y/T36I/V45A/V49M, M1V/L2S/M4T/N11D/H19Y/T36A, M1V/L2S/M4T/S9P/Q10R/H19Y/T36A/T46A/N47S, M1V/L2S/M4T/S16G/N31D/T32I/T36A, M1V/M4T/T36A/Q38R/N53K, M1T/N11D/H19Y/T36A/N42S/V45A/N53S, M1T/N31D/T32A/T36A/A38R/S44D/V49A/K50E, M1T/S9P/P23S/Q38R/N42S/S48P/V49A/A54V, H19Y/T36A/S44G, H19Y/T36A, and M4T/T36A/Q38R/N42S/S44G/T46A/N47K/S48P/T52A.

In an additional aspect, the invention provides compositions as described herein, wherein said variant sBCMA comprises the amino acid substitutions S16G/H19Y/T36A, and at least one further amino acid substitution selected from the group consisting of M1A, M1C, M1I, M1R, M1T, M1V, L2C, L2S, Q3P, Q3R, M4E, M4I, M4T, M4V, A5T, G6E, Q7R, S9A, S9F, S9P, Q10H, Q10P, Q10R, N11D, N11S, E12K, F14L, S16N, S16R, H19L, A20V, A20T, I22M, I22V, P23S, Q25R, L26F, S29A, N31D, N31S, T32A, T32I, T32P, L35S, L35P, T36I, T36P, Q38R, R39H, N42D, N42R, N42S, A43T, A43V, S44D, S44G, S44N, S44R, V45A, V45M, T46A, T46I, N47D, N47K, N47R, N47S, S48L, S48P, S48T, V49A, V49M, K50E, K50G, K50R, K50T, G51E, T52A, T52M, N53D, N53K, N53S, A54V, and A54T.

In a further aspect, the invention provides compositions as described herein, wherein said variant sBCMA comprises the amino acid substitutions S16G/H19Y/T36A/N53D, and at least one further amino acid substitution selected from the group consisting of M1A, M1C, M1I, M1R, M1T, M1V, L2C, L2S, Q3P, Q3R, M4E, M4I, M4T, M4V, A5T, G6E, Q7R, S9A, S9F, S9P, Q10H, Q10P, Q10R, N11D, N11S, E12K, F14L, S16N, S16R, H19L, A20V, A20T, I22M, I22V, P23S, Q25R, L26F, S29A, N31D, N31S, T32A, T32I, T32P, L35S, L35P, T36I, T36P, Q38R, R39H, N42D, N42R, N42S, A43T, A43V, S44D, S44G, S44N, S44R, V45A, V45M, T46A, T46I, N47D, N47K, N47R, N47S, S48L, S48P, S48T, V49A, V49M, K50E, K50G, K50R, K50T, G51E, T52A, T52M, N53K, N53S, A54V, and A54T.

In an additional aspect, the invention provides compositions as described herein, wherein said variant sBCMA comprises the amino acid substitutions S9P/N11D/S16G/H19Y/T36A/N47S/N53D, and at least one further amino acid substitution selected from the group consisting of M1A, MIC, M1I, M1R, M1T, M1V, L2C, L2S, Q3P, Q3R, M4E, M4I, M4T, M4V, A5T, G6E, Q7R, S9A, S9F, Q10H, Q10P, Q10R, N11S, E12K, F14L, S16N, S16R, H19L, A20V, A20T, I22M, I22V, P23S, Q25R, L26F, S29A, N31D, N31S, T32A, T32I, T32P, L35S, L35P, T36I, T36P, Q38R, R39H, N42D, N42R, N42S, A43T, A43V, S44D, S44G, S44N, S44R, V45A, V45M, T46A, T46I, N47D, N47K, N47R, S48L, S48P, S48T, V49A, V49M, K50E, K50G, K50R, K50T, G51E, T52A, T52M, N53K, N53S, A54V, and A54T.

In a further aspect, the invention provides compositions as described herein, wherein said variant sBCMA comprises the amino acid substitutions Q3P/S9P/H19Y/N31S/T36A/R39H/N47R/K50E, and at least one further amino acid substitution selected from the group consisting of M1A, M1C, M1I, M1R, M1T, M1V, L2C, L2S, Q3R, M4E, M4I, M4T, M4V, A5T, G6E, Q7R, S9A, S9F, Q10H, Q10P, Q10R, N11D, N11S, E12K, F14L, S16G, S16N, S16R, H19L, A20V, A20T, I22M, I22V, P23S, Q25R, L26F, S29A, N31D, T32A, T32I, T32P, L35S, L35P, T36I, T36P, Q38R, N42D, N42R, N42S, A43T, A43V, S44D, S44G, S44N, S44R, V45A, V45M, T46A, T46I, N47D, N47K, N47S, S48L, S48P, S48T, V49A, V49M, K50G, K50R, K50T, G51E, T52A, T52M, N53D, N53K, N53S, A54V, and A54T.

In an additional aspect, the invention provides compositions as described herein, wherein said variant sBCMA comprises the amino acid substitutions M1V/L2S/M4T/S16G/N31D/T32I/T36A, and at least one further amino acid substitution selected from the group consisting of M1A, M1C, M1I, M1R, M1T, L2C, Q3P, Q3R, M4E, M4I, M4V, A5T, G6E, Q7R, S9A, S9F, S9P, Q10H, Q10P, Q10R, N11D, N11S, E12K, F14L, S16N, S16R, H19L, H19Y, A20V, A20T, I22M, I22V, P23S, Q25R, L26F, S29A, N31S, T32A, T32P, L35S, L35P, T36I, T36P, Q38R, R39H, N42D, N42R, N42S, A43T, A43V, S44D, S44G, S44N, S44R, V45A, V45M, T46A, T46I, N47D, N47K, N47R, N47S, S48L, S48P, S48T, V49A, V49M, K50E, K50G, K50R, K50T, G51E, T52A, T52M, N53D, N53K, N53S, A54V, and A54T.

In a further aspect, the invention provides compositions as described herein, wherein said variant sBCMA has at least 90% sequence identity to SEQ ID NO: 67.

In an additional aspect, the invention provides compositions as described herein, wherein said variant sBCMA has at least 90% sequence identity to SEQ ID NO: 68.

In a further aspect, the invention provides compositions as described herein, wherein said variant sBCMA has at least 90% sequence identity to SEQ ID NO: 69.

In an additional aspect, the invention provides compositions as described herein, wherein said variant sBCMA has at least 90% sequence identity to SEQ ID NO: 49.

In a further aspect, the invention provides compositions as described herein, wherein said variant sBCMA has at least 90% sequence identity to SEQ ID NO: 74.

In an additional aspect, the invention provides compositions as described herein, wherein said variant sBCMA has the amino acid sequence of SEQ ID NO: 67.

In a further aspect, the invention provides compositions as described herein, wherein said variant sBCMA has the amino acid sequence of SEQ ID NO: 68.

In an additional aspect, the invention provides compositions as described herein, wherein said variant sBCMA has the amino acid sequence of SEQ ID NO: 69.

In a further aspect, the invention provides compositions as described herein, wherein said variant sBCMA has the amino acid sequence of SEQ ID NO: 49.

In an additional aspect, the invention provides compositions as described herein, wherein said variant sBCMA has the amino acid sequence of SEQ ID NO: 74.

In a further aspect, the invention provides compositions as described herein, wherein said variant sBCMA exhibits enhanced binding affinity for A Proliferation Inducing Ligand (APRIL) or B-cell Activating Factor of the TNF family (BAFF) as compared to SEQ ID NO:1.

In an additional aspect, the invention provides compositions as described herein, wherein said variant sBCMA exhibits enhanced binding affinity for APRIL and BAFF as compared to SEQ ID NO:1.

In a further aspect, the invention provides nucleic acids encoding said variant sBCMA proteins as described herein.

In an additional aspect, the invention provides expression vectors comprising the nucleic acids as described herein.

In a further aspect, the invention provides host cells comprising the nucleic acids or expression vectors as described herein.

In an additional aspect, the invention provides a method of making a variant sBCMA protein comprising: a) culturing the host cell as described herein under conditions wherein said Fc fusion protein is expressed; and b) recovering said variant sBCMA protein.

In a further aspect, the invention provides a composition comprising an sBCMA variant—Fc fusion protein comprising:
a) a variant sBCMA domain comprising at least one amino acid substitution as compared to SEQ ID NO:1, wherein said amino acid substitution is at a position number selected from the group consisting of 1, 2, 3, 4, 5, 6, 7, 9, 10, 11, 12, 14, 16, 19, 20, 22, 23, 25, 26, 29, 31, 32, 35, 36, 38, 39, 42, 43, 44, 45, 46, 47, 48, 49, 50, 51, 52, 53, and 54, wherein the numbering is according to the EU index;
b) an optional linker; and
c) an Fc domain.

In an additional aspect, the invention provides the composition as described herein, wherein said fusion protein comprises, from N- to C-terminal:
a) said variant sBCMA domain;
b) said optional linker; and
c) said Fc domain.

In a further aspect, the invention provides the composition as described herein, wherein said fusion protein comprises, from N- to C-terminal:
a) said Fc domain;
b) said optional linker; and
c) said variant sBCMA domain.

In an additional aspect, the invention provides the composition as described herein, wherein said variant sBCMA domain has at least 80%, at least 85%, at least 90%, or at least 95% sequence identity to SEQ ID NO:1.

In a further aspect, the invention provides the composition as described herein, wherein said amino acid substitution(s) occur at one of said positions, two of said positions, three of said positions, four of said positions, five of said positions, six of said positions, seven of said positions, eight of said positions, or nine of said positions.

In an additional aspect, the invention provides the composition as described herein, wherein said amino acid substitution(s) is selected from the group consisting of M1A, M1C, M1I, M1R, M1T, M1V, L2C, L2S, Q3P, Q3R, M4E, M4I, M4T, M4V, A5T, G6E, Q7R, S9A, S9F, S9P, Q10H, Q10P, Q10R, N11D, N11S, E12K, F14L, S16G, S16N, S16R, H19L, H19Y, A20V, A20T, I22M, I22V, P23S, Q25R, L26F, S29A, N31D, N31S, T32A, T32I, T32P, L35S, L35P, T36A, T36I, T36P, Q38R, R39H, N42D, N42R, N42S, A43T, A43V, S44D, S44G, S44N, S44R, V45A, V45M, T46A, T46I, N47D, N47K, N47R, N47S, S48L, S48P, S48T, V49A, V49M, K50E, K50G, K50R, K50T, G51E, T52A, T52M, N53D, N53K, N53S, A54V, and A54T.

In a further aspect, the invention provides the composition as described herein, wherein said amino acid substitution(s) is selected from the group consisting of M1V, L2S, Q3P, M4T, S9P, N11D, S16G, H19Y, N31S, N31D, T32I, T36A, R39H, N47S, K50E, and N53E.

In an additional aspect, the invention provides the composition as described herein, wherein said amino acid substitution(s) is selected from the group consisting of S16G, H19Y and T36A.

In a further aspect, the invention provides the composition as described herein, wherein said amino acid substitutions are selected from the group consisting of L2S/S9P/E12K/N31D/T36A/N42S/N53S, M1V/T32P/T36A/T46I/N53D/A54V, Q3R/S16N/T36A/A43T, F14L/S16G/T36A/V45A/N47D, M1T/M4V/S9F/S16G/T32A/Q38R, M1A/S9A/Q38R, G6E/Q25R/Q38R, M1V/M4I/G6E/S9P/N11D/V49M/T52M/A54V, N11D/S16G/N31S, N11D/H19Y/I22M/T32P/N47S/N53S, G6E/Q7R/H19Y/L35S, H19Y/N42D/S48P/T52A, M1V/N31D/T32I/T36A, M1V/A5T/H19L/T36A, M1T/N31D/T32A/T36A/Q38R/S44D/V49A/K50E, M1V/T36A/Q38R/A43V, M1V/L2S/S9P/Q10H/T36A/Q38R/K50G, T36A/Q38R/N53S, M1T/L2S/L35P/T36A/Q38R/T46A/K50R, A5T/A20V/T36A/Q38R, M1T/S16G/I22V/T36A/S44G/T46A/V49A, S16G/T36A, M1I/N11D/S16G/I22M/S29A/T36A/S44G/K50R, M1C/L2C/Q3R/M4E/N11D/S16G/T36P, M1I/N11D/S16G/I22M/S29A/T36A/S44G/K50R, N11D/N31D/T32I/T36A/S44N/N47D/N53D, M1R/L2C/Q3R, H19Y/T36A/S44G, H19Y/T32I/T36A/V49A, H19Y/N31S/T36A/V45A, H19Y/N31S/T36A, H19Y/T36P/T52A, H19Y/N31D/T52M, M1V/H19Y/V45M, S16G/H19Y/N47D, S16G/H19Y/K50T, S16G/H19Y/S44N/K50R, N11D/H19Y/S48T, S9P/N11D/S16R/T32A/Q38R/S44G/T46I/T52A/N53D/A54T, N11D/S16G/S44R, H19L/T32A/S44G/G51E/T52A, S16N/H19Y/T36A/K50R, M1V/H19Y/T36A/R39H/T46A, M1V/H19Y/T36A, H19Y/T36A/N42D/N47S/S48P, M1V/H19Y/T36A/S44G/N47D, M1V/H19Y/T36A/N42R/N53S, H19Y/L35P/T36A/N42D/T46I/V49A, Q3P/S9P/H19Y/N31S/T36A/R39H/N47R/K50E, M1V/H19Y/T36A/N42R/N53S, M1T/H19Y/T36A, M1V/S16N/H19Y/I22M/T36A, M1T/N11D/H19Y/T36A/N42S/V45A/N53S, N11D/S16G/H19Y/T36A/N47S/N53D, M1V/S9P/Q10P/S16G/H19Y/L26F/T36A/A43V/N53D, S16G/H19Y/T36A/V49A/N53D, S16G/T36A/A43T/S44G/V45M, M4V/S9P/S16G/T36A/Q38R, S9P/N11S/S16G/T36A/Q38R, N11D/E12K/S16R/T36A/T52M, M4V/T32I/T36A/Q38R/A43T/V45A/S48P, S9P/N11D/S16G/Q25R, M1T/A5T/S9P/S16G/Q25R/N31D/V49M, L2S/S9P/S16G/A20T/T32I/Q38R/N42D/T46A/S48L, S16G/Q25R/T46A, G6E/S9A/S16G/Q25R/N31D/N47S/T52M, H19Y/Q38R/T52M, N11D/H19Y/I22M/T32P/N47S/N53S, S16G/H19Y/T36A, S16G/H19Y/T36A/N53D, S9P/N11D/S16G/H19Y/T36A/N47S/N53D, Q3P/S9P/H19Y/N31S/T36A/R39H/N47R/K50E, M1V/L2S/M4T/N11D/H19Y/T36A, M1V/L2S/M4T/N11D/T36A, M1V/L2S/M4T/H19Y/T36I/V45A/V49M, M1V/L2S/M4T/N11D/H19Y/T36A, M1V/L2S/M4T/S9P/Q10R/H19Y/T36A/T46A/N47S, M1V/L2S/M4T/S16G/N31D/T32I/T36A, M1V/M4T/T36A/Q38R/N53K, M1T/N11D/H19Y/T36A/N42S/V45A/N53S, M1T/N31D/T32A/T36A/A38R/S44D/V49A/K50E, M1T/S9P/P23S/Q38R/N42S/S48P/V49A/A54V, H19Y/T36A/S44G, H19Y/T36A, and M4T/T36A/Q38R/N42S/S44G/T46A/N47K/S48P/T52A.

In an additional aspect, the invention provides the composition as described herein, wherein said variant sBCMA domain comprises the amino acid substitutions S16G/H19Y/T36A, and at least one further amino acid substitution selected from the group consisting of M1A, M1C, M1I, M1R, M1T, M1V, L2C, L2S, Q3P, Q3R, M4E, M4I, M4T, M4V, A5T, G6E, Q7R, S9A, S9F, S9P, Q10H, Q10P, Q10R, N11D, N11S, E12K, F14L, S16N, S16R, H19L, A20V, A20T, I22M, I22V, P23S, Q25R, L26F, S29A, N31D, N31S, T32A, T32I, T32P, L35S, L35P, T36I, T36P, Q38R, R39H, N42D, N42R, N42S, A43T, A43V, S44D, S44G, S44N, S44R, V45A, V45M, T46A, T46I, N47D, N47K, N47R, N47S, S48L, S48P, S48T, V49A, V49M, K50E, K50G, K50R, K50T, G51E, T52A, T52M, N53D, N53K, N53S, A54V, and A54T.

In a further aspect, the invention provides the composition as described herein, wherein said variant sBCMA domain comprises the amino acid substitutions S16G/H19Y/T36A/N53D, and at least one further amino acid substitution selected from the group consisting of M1A, M1C, M1I, M1R, M1T, M1V, L2C, L2S, Q3P, Q3R, M4E, M4I, M4T, M4V, A5T, G6E, Q7R, S9A, S9F, S9P, Q10H, Q10P, Q10R, N11D, N11S, E12K, F14L, S16N, S16R, H19L, A20V, A20T, I22M, I22V, P23S, Q25R, L26F, S29A, N31D, N31S, T32A, T32I, T32P, L35S, L35P, T36I, T36P, Q38R, R39H, N42D, N42R, N42S, A43T, A43V, S44D, S44G, S44N, S44R, V45A, V45M, T46A, T46I, N47D, N47K, N47R, N47S, S48L, S48P, S48T, V49A, V49M, K50E, K50G, K50R, K50T, G51E, T52A, T52M, N53K, N53S, A54V, and A54T.

In an additional aspect, the invention provides the composition as described herein, wherein said variant sBCMA domain comprises the amino acid substitutions S9P/N11D/S16G/H19Y/T36A/N47S/N53D, and at least one further amino acid substitution selected from the group consisting of M1A, MIC, M1I, M1R, M1T, M1V, L2C, L2S, Q3P, Q3R, M4E, M4I, M4T, M4V, A5T, G6E, Q7R, S9A, S9F, Q10H, Q10P, Q10R, N11S, E12K, F14L, S16N, S16R, H19L, A20V, A20T, I22M, I22V, P23S, Q25R, L26F, S29A, N31D, N31S, T32A, T32I, T32P, L35S, L35P, T36I, T36P, Q38R, R39H, N42D, N42R, N42S, A43T, A43V, S44D, S44G, S44N, S44R, V45A, V45M, T46A, T46I, N47D, N47K, N47R, S48L, S48P, S48T, V49A, V49M, K50E, K50G, K50R, K50T, G51E, T52A, T52M, N53K, N53S, A54V, and A54T.

In a further aspect, the invention provides the composition as described herein, wherein said variant sBCMA domain comprises the amino acid substitutions Q3P/S9P/H19Y/N31S/T36A/R39H/N47R/K50E, and at least one further amino acid substitution selected from the group consisting of M1A, M1C, M1I, M1R, M1T, M1V, L2C, L2S, Q3R, M4E, M4I, M4T, M4V, A5T, G6E, Q7R, S9A, S9F, Q10H, Q10P, Q10R, N11D, N11S, E12K, F14L, S16G, S16N, S16R, H19L, A20V, A20T, I22M, I22V, P23S, Q25R, L26F, S29A, N31D, T32A, T32I, T32P, L35S, L35P, T36I, T36P, Q38R, N42D, N42R, N42S, A43T, A43V, S44D, S44G, S44N, S44R, V45A, V45M, T46A, T46I, N47D, N47K, N47S, S48L, S48P, S48T, V49A, V49M, K50G, K50R, K50T, G51E, T52A, T52M, N53D, N53K, N53S, A54V, and A54T.

In an additional aspect, the invention provides the composition as described herein, wherein said variant sBCMA domain comprises the amino acid substitutions M1V/L2S/M4T/S16G/N31D/T32I/T36A, and at least one further amino acid substitution selected from the group consisting of M1A, M1C, M1I, M1R, M1T, L2C, Q3P, Q3R, M4E, M4I, M4V, A5T, G6E, Q7R, S9A, S9F, S9P, Q10H, Q10P, Q10R, N11D, N11S, E12K, F14L, S16N, S16R, H19L, H19Y, A20V, A20T, I22M, I22V, P23S, Q25R, L26F, S29A, N31S, T32A, T32P, L35S, L35P, T36I, T36P, Q38R, R39H, N42D, N42R, N42S, A43T, A43V, S44D, S44G, S44N, S44R, V45A, V45M, T46A, T46I, N47D, N47K, N47R, N47S, S48L, S48P, S48T, V49A, V49M, K50E, K50G, K50R, K50T, G51E, T52A, T52M, N53D, N53K, N53S, A54V, and A54T.

In a further aspect, the invention provides the composition as described herein, wherein said variant sBCMA domain has at least 90% sequence identity to SEQ ID NO: 67.

In an additional aspect, the invention provides the composition as described herein, wherein said variant sBCMA domain has at least 90% sequence identity to SEQ ID NO: 68.

In a further aspect, the invention provides the composition as described herein, wherein said variant sBCMA domain has at least 90% sequence identity to SEQ ID NO: 69.

In an additional aspect, the invention provides the composition as described herein, wherein said variant sBCMA domain has at least 90% sequence identity to SEQ ID NO: 49.

In a further aspect, the invention provides the composition as described herein, wherein said variant sBCMA domain has at least 90% sequence identity to SEQ ID NO: 74.

In an additional aspect, the invention provides the composition as described herein, wherein said variant sBCMA domain has the amino acid sequence of SEQ ID NO: 67.

In a further aspect, the invention provides the composition as described herein, wherein said variant sBCMA domain has the amino acid sequence of SEQ ID NO: 68.

In an additional aspect, the invention provides the composition as described herein, wherein said variant sBCMA domain has the amino acid sequence of SEQ ID NO: 69.

In a further aspect, the invention provides the composition as described herein, wherein said variant sBCMA domain has the amino acid sequence of SEQ ID NO: 49.

In an additional aspect, the invention provides the composition as described herein, wherein said variant sBCMA domain has the amino acid sequence of SEQ ID NO: 74.

In a further aspect, the invention provides the composition as described herein, wherein said variant sBCMA domain exhibits enhanced binding affinity for APRIL or BAFF as compared to SEQ ID NO: 1.

In an additional aspect, the invention provides the composition as described herein, wherein said variant sBCMA domain exhibits enhanced binding affinity for APRIL and BAFF as compared to SEQ ID NO:1.

In a further aspect, the invention provides the composition as described herein, wherein said Fc domain is a human IgG Fc domain or a variant human IgG Fc domain.

In an additional aspect, the invention provides the composition as described herein, wherein said human IgG Fc domain comprises the hinge-CH2-CH3 of human IgG1.

In a further aspect, the invention provides the composition as described herein, wherein said Fc domain is a variant human IgG Fc domain.

In a further aspect, the invention provides the composition as described herein, wherein said linker is IEGRMD (SEQ ID NO:87).

In an additional aspect, the invention provides the composition as described herein, wherein said linker is selected from the group consisting of (GS)n, (GSGGS)n, (GGGGS)n, and (GGGS)n, wherein n is selected from the group consisting of 1, 2, 3, 4 and 5.

In a further aspect, the invention provides the composition as described herein, wherein said linker is GGGGS (SEQ ID NO:88).

In an additional aspect, the invention provides the composition as described herein, wherein the sBCMA variant—Fc fusion protein has the amino acid sequence of SEQ ID NO:80.

In a further aspect, the invention provides the composition as described herein, wherein the sBCMA variant—Fc fusion protein has the amino acid sequence of SEQ ID NO:81.

In an additional aspect, the invention provides the composition as described herein, wherein the sBCMA variant—Fc fusion protein has the amino acid sequence of SEQ ID NO:82.

In a further aspect, the invention provides the composition as described herein, wherein the sBCMA variant—Fc fusion protein has the amino acid sequence of SEQ ID NO:83.

In an additional aspect, the invention provides the composition as described herein, wherein the sBCMA variant—Fc fusion protein has the amino acid sequence of SEQ ID NO:84.

In an additional aspect, the invention provides a nucleic acid encoding said fusion protein as described herein.

In a further aspect, the invention provides an expression vector comprising said nucleic acid as described herein.

In an additional aspect, the invention provides a host cell comprising said nucleic acid as described herein or said expression vector as described herein.

In a further aspect, the invention provides a method of making an sBCMA variant—Fc fusion protein comprising: a) culturing said host cell as described herein under conditions wherein said fusion protein is expressed; and b) recovering said fusion protein.

In an additional aspect, the invention provides a method of treating, reducing or preventing metastasis or invasion of a tumor that expresses APRIL in a subject, said method comprising administering to the subject a therapeutically effective dose of one or more said fusion proteins as described herein. In some embodiments, the tumor as disclosed herein is a hematologic cancer. In some embodiments, the hematologic cancer as disclosed herein is multiple myeloma.

In a further aspect, the invention provides a method of inhibiting the activity of APRIL in a subject having a tumor that expresses APRIL, said method comprising administering to the subject a therapeutically effective dose of one or more said fusion proteins as described herein. In some embodiments, the tumor as disclosed herein is a hematologic cancer. In some embodiments, the hematologic cancer as disclosed herein is multiple myeloma.

In an additional aspect, the invention provides a method of inhibiting B-cell growth, immunoglobulin production, or both in a subject, said method comprising administering to the subject a therapeutically effective dose of one or more said fusion proteins as described herein, wherein said variant sBCMA domain binds to BAFF.

In a further aspect, the invention provides a method of treating an autoimmune disease expressing at least one receptor selected from the group consisting of BCMA, BAFFR, TACI and other receptor(s) that are activated through binding to BAFF in a subject, said method comprising administering to the subject a therapeutically effective dose of one or more said fusion proteins as described herein.

In an additional aspect, the invention provides a method of treating an autoimmune disease expressing BAFF and/or APRIL in a subject, said method comprising administering to the subject a therapeutically effective dose of one or more said fusion proteins as described herein.

In an additional aspect, the invention provides a method of treating fibrosis expressing BCMA, BAFFR and/or TACI in a subject, said method comprising administering to the subject a therapeutically effective dose of one or more said fusion proteins as described herein.

In a further aspect, the invention provides a method of treating fibrosis expressing BAFF and/or APRIL in a subject, said method comprising administering to the subject a therapeutically effective dose of one or more said fusion proteins as described herein.

### IV. BRIEF DESCRIPTION OF THE DRAWINGS

FIG. 1 shows the binding kinetics of WT-BCMA to APRIL. Binding assay for WT-BCMA binding to APRIL shows a Kd of 32 pM.

FIG. 2 shows various sorting conditions and gates for BCMA yeast display library.

FIGS. 3A-3D show the sequences of sBCMA variant clones as compared to the sequence of the extracellular domain of wild-type human BCMA as set forth in SEQ ID NO:1. FIG. 3A shows the sequences of S3 clones #1-12. FIG. 3B shows the sequences of S4 clones #13-41. FIG. 3C shows the sequences of S5 clones #42-74. FIG. 3D shows the sequences of S6 clones #75-118.

FIG. 4 shows binding curves for various sBCMA variant clones to APRIL.

FIG. 5 shows binding curves for various sBCMA variant clones to BAFF.

FIG. 6 shows the amino acid sequences of sBCMA variant—Fc fusion proteins as set forth in SEQ ID NO:80, SEQ ID NO:81, SEQ ID NO:82, SEQ ID NO:83, and SEQ ID NO:84. The variant sBCMA domain is underlined, the linker domain is bolded and the human IgG1 Fc domain is italic.

FIG. 7 shows the amino acid sequences of the extracellular domain of wild type human BCMA (SEQ ID NO:1), the extracellular domain of APRIL (SEQ ID NO:85), the extracellular domain of BAFF (SEQ ID NO:86), a linker domain (SEQ ID NO:87) and another linker domain (SEQ ID NO:88).

FIGS. 8A and 8B show tumor growth curves and terminal tumor weights in the tumor efficacy study based on the MM1.R Multiple Myeloma mouse model (Example 5). FIG. 8A shows tumor growth curves of MM1.R Multiple Myeloma mouse models treated with 1 mg/kg and 10 mg/kg of sBCMA variant—Fc fusion protein or 1 mg/kg and 10 mg/kg of wild type sBCMA Fc. FIG. 8B shows terminal tumor weights in each of the treated groups.

### V. DETAILED DESCRIPTION OF THE INVENTION

In order to more clearly and concisely point out the subject matter of the claimed invention, the following definitions are provided for specific terms used in the following written description and appended claims.

### A. Introduction

The present invention is directed to the use of soluble forms of human BCMA that contain amino acid modifications, e.g. variant sBCMA proteins. These variant sBCMA proteins bind to either one or both of the BCMA ligands, human BAFF and/or human APRIL, with tighter affinity than wild type human BCMA. APRIL and BAFF can bind to receptors, such as BCMA, BAFFR and TACI, and thus neutralizing APRIL and/or BAFF can be used for treating diseases arising from altered signaling pathways through BCMA, BAFFR and/or TAC. These diseases include cancers, autoimmune diseases and fibrosis. Neutralizing APRIL alone can be effective in treating cancers, autoimmune diseases and fibrosis expressing high levels of BCMA and TACI or other receptors that are activated through binding to APRIL. Neutralizing BAFF alone can be effective in treating B cell malignancies and autoimmune diseases expressing BCMA, BAFFR and TACI or other receptors that are activated through binding to BAFF. Therefore, the variant sBCMA as described herein can be used to treat cancers, immunomodulatory disorders and/or fibrotic diseases expressing BCMA, BAFFR, TACI and/or any other receptors that are activated through binding to APRIL and/or BAFF by binding more tightly, and thus preferentially, to the ligand(s) e.g. APRIL and/or BAFF and thus altering the normal receptor signaling that would otherwise occur between BCMA, BAFFR and/or TACI on the surface of a cell with APRIL or BAFF.

Additionally, in some embodiments, since the sBCMA variants are small proteins that generally are cleared rapidly from the bloodstream, the invention provides fusion proteins that link the sBCMA variant to a human or variant Fc domain as discussed herein. Since Fc domains, through binding to the FcRn receptor, confer extended half-life in serum, the creation of an sBCMA variant-Fc domain fusion proteins results in improved therapies. Thus, the invention provides sBCMA domain-Fc domain fusion proteins, referred sometimes herein as “fusion proteins”. In some embodiments, the sBCMA variant or the variant sBCMA domain of the fusion protein as described herein exhibits enhanced binding affinity for APRIL as compared to SEQ ID NO:1. In some embodiments, the sBCMA variant or the variant sBCMA domain of the fusion protein as described herein exhibits enhanced binding affinity for BAFF as compared to SEQ ID NO:1. In some embodiments, the sBCMA variant or the variant sBCMA domain of the fusion protein as described herein exhibits enhanced binding affinity for APRIL and BAFF as compared to SEQ ID NO:1.

### B. Definitions

As used herein, the following terms have the meanings ascribed to them unless specified otherwise.

The terms “a”, “an”, or “the” as used herein not only include aspects with one member, but also include aspects with more than one member. For instance, the singular forms “a”, “an”, and “the” include plural referents unless the context clearly dictates otherwise. Thus, for example, reference to “a cell” includes a plurality of such cells and reference to “the agent” includes reference to one or more agents known to those skilled in the art, and so forth.

As used herein, “protein” herein is meant at least two covalently attached amino acids, which includes proteins, polypeptides, oligopeptides and peptides.

The term “isolated” refers to a molecule that is substantially free of its natural environment and devoid of other proteins. For instance, an isolated protein is substantially free of cellular material or other proteins from the cell or tissue source from which it is derived. The term “isolated” also refers to preparations where the isolated protein is sufficiently pure to be administered as a pharmaceutical composition, or at least about 70-80%, 80-90%, or 90-95% (w/w) pure, or at least about 95%, 96%, 97%, 98%, 99%, or 100% (w/w) pure. In particular, it is preferred that the polypeptides are in “essentially pure form”, i.e., that the polypeptide preparation is essentially free of other polypeptide material with which it is natively associated. This can be accomplished, for example, by preparing the polypeptide by means of well-known recombinant methods or by classical purification methods.

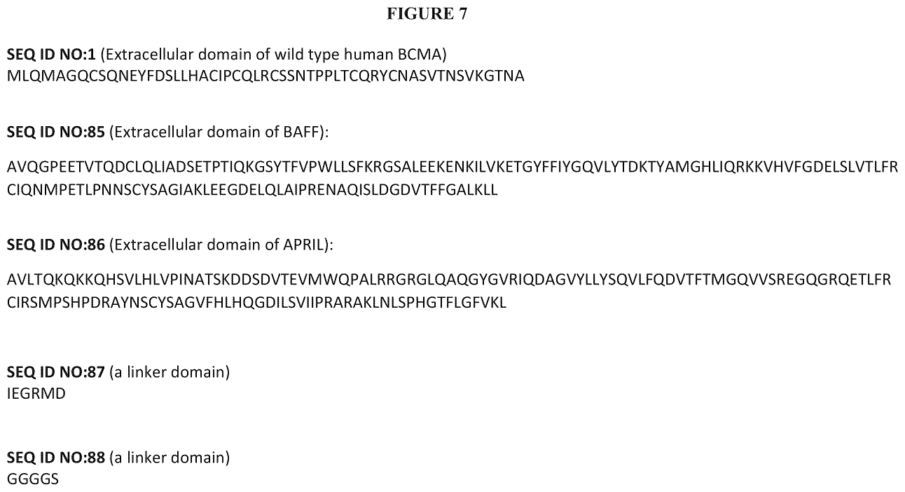

The term B-cell maturation antigen “BCMA” refers to the protein for B cell maturation as described in Gras et al. International Immunology, 1995, 7:1093; Y. Laabi et al. EMBO J, 1992, 11:3897. BCMA is a member of the TNF-receptor superfamily. For example, BCMA is a receptor for APRIL and BAFF. The amino acid sequence of the extracellular domain of the wild type human BCMA (SEQ ID NO:1) is shown in FIG. 7 and Table 3.

The term “ligand” refers to a biomolecule that is able to bind to and form a complex with a second biomolecule such as a receptor present on the surface of target cells to serve a biological purpose. A ligand is generally an effector molecule that binds to a site on a target protein, e.g., by intermolecular forces such as ionic bonds, hydrogen bonds, hydrophobic interactions, dipole-dipole bonds, or Van der Waals forces. In the present invention, APRIL and BAFF are ligand proteins.

The term “receptor” refers to a biomolecule present on the surface of a target cell that is able to bind to and form a complex with a second biomolecule such as a ligand. A receptor generally activates a specific signal transduction pathway. For example, BCMA is a receptor for APRIL and BAFF, members of the TNF family.

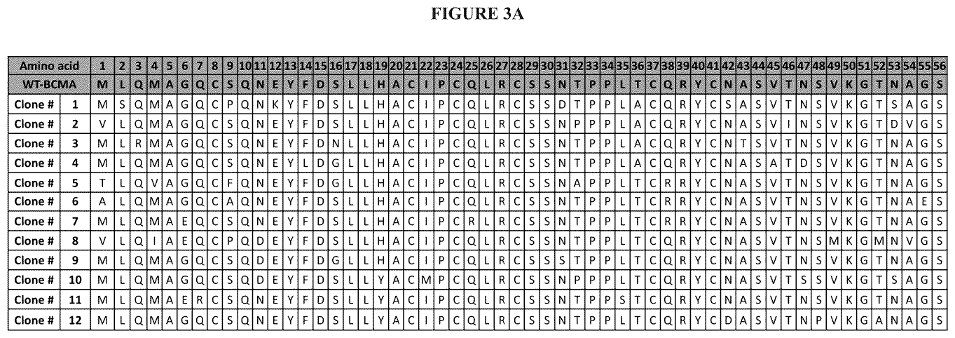

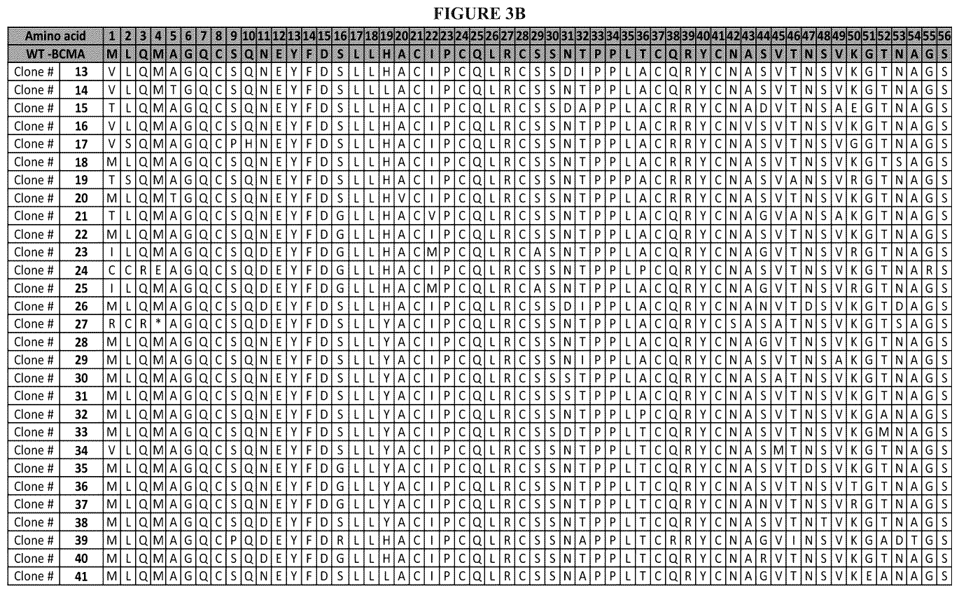

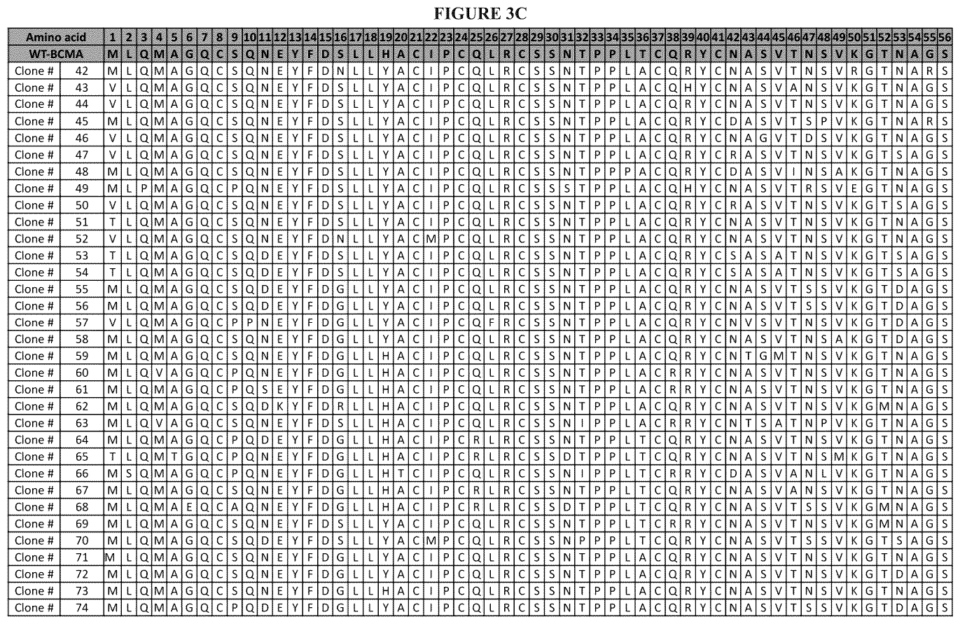

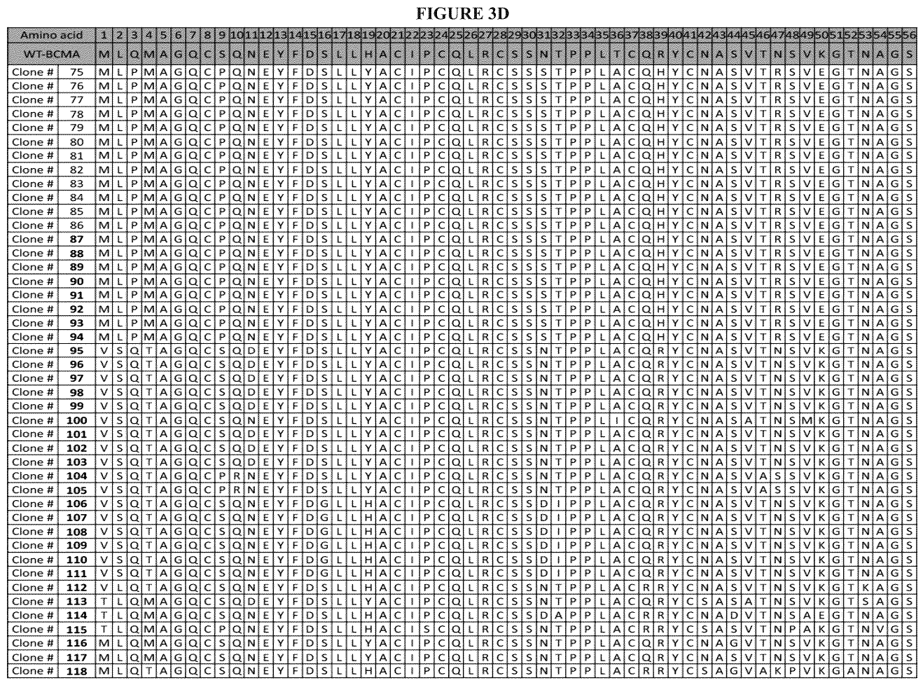

By “position” as used herein is meant a location in the sequence of a protein. In some embodiments of the present invention, positions are numbered sequentially starting with the first amino acid of the mature protein, for example for the human BCMA protein shown in FIG. 3. In some cases, for example for the Fc domain portion of the fusion proteins described herein, the Fc domain positions may be numbered sequentially, or according to an established format, for example the EU index. The EU index or EU index as in Kabat or EU numbering scheme refers to the EU numbering (see SEQUENCES OF IMMUNOLOGICAL INTEREST, 5th edition, NIH publication, No. 91-3242, E. A. Kabat et al., entirely incorporated by reference; and see also Edelman et al., 1969, Proc Natl Acad Sci USA 63:78-85, hereby entirely incorporated by reference).

By “amino acid modification” or “amino acid sequence modification” herein is meant an amino acid substitution, insertion, and/or deletion in a polypeptide sequence.

By “parent protein” as used herein is meant a starting protein that is subsequently modified to generate a variant. The parent protein may be a naturally occurring protein, or a variant or engineered version of a naturally occurring protein. Parent protein may refer to the protein itself, compositions that comprise the parent protein, or the amino acid sequence that encodes it. In this context, a “parent Fc domain” will be relative to the recited variant; thus, a “variant human IgG Fc domain” is compared to the parent Fc domain of human IgG, for example, a “variant human IgG1 Fc domain” is compared to the parent Fc domain of human IgG1, etc.

By “wild type” or “WT” herein is meant an amino acid sequence or a nucleotide sequence that is found in nature, including allelic variations. A WT protein has an amino acid sequence or a nucleotide sequence that has not been intentionally modified into a non-naturally occurring sequence.

By “variant protein” or “protein variant”, or “variant” as used herein is meant a protein with an amino acid sequence which differs from that of a parent protein by virtue of at least one amino acid sequence modification. For example, “variant sBCMA” or “sBCMA variant” as used herein is meant a protein with an amino acid sequence which differs from that of a parent sBCMA protein by virtue of at least one amino acid sequence modification yet still retains the ability to bind to a cognate ligand, as outlined below. In some embodiments, the parent proteins are human wild type sequences. In some embodiments, the parent proteins are human sequences with variants. Protein variant may refer to the protein itself, a composition comprising the protein, or the amino sequence that encodes it. In some embodiments, the protein variant has amino acid substitution(s) at one position, two positions, three positions, four positions, five positions, six positions, seven positions, eight positions, nine positions or ten positions. The protein variant sequence herein will possess at least about 80%, 85%, 90%, 91%, 92%, 93%, 94%, 95%, 96%, 97%, or 98% sequence identity with a parent protein sequence, and preferably at least about 85%, 86%, 88%, 90%, 93% or 95% sequence identity. The relatedness between two amino acid sequences or between two nucleotide sequences is described by the parameter “sequence identity” or “identity”. The degree of identity between an amino acid sequence of the present invention (“invention sequence”) and the parent amino acid sequence referred to in the claims (e.g. SEQ ID NO:1) is calculated as the number of exact matches in an alignment of the two sequences, divided by the length of the “invention sequence,” or the length of the parent amino acid sequence, whichever is the shortest. The result is expressed in percent identity as calculated below.

For purposes of the present invention, the extracellular domain of sBCMA as set forth in SEQ ID NO:1 is used as a parent protein to determine the corresponding amino acid sequence modification in sBCMA variants of the present invention. The amino acid sequence of an sBCMA variant protein is aligned with the amino acid sequence of SEQ ID NO:1, and based on the alignment, the amino acid position number corresponding to any amino acid residue as disclosed in SEQ ID NO:1 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

Identification of the corresponding amino acid residue in another sBCMA variant can be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 51 1-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), EMBOSS EMMA employing ClustalW (1.83 or later; Thompson et al., 1994, Nucleic Acids Research 22: 4673-4680), and EMBL-EBI employing Clustal Omega (Sievers and Higgins, 2014, Methods Mol Biol. 2014; 1079:105-16), using their respective default parameters.

When the other variant polypeptides have diverged from the wild type sBCMA such that traditional sequence-based comparison fails to detect their relationship (Lindahl and Elofsson, 2000, J. Mol. Biol. 295: 613-615), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example, the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homologs (e.g., Holm and Park, 2000, Bioinformatics 16: 566-567).

In describing the variants of the present invention, the nomenclature described below is adapted for ease of reference. The standardly accepted IUPAC single letter or three letter amino acid abbreviation is employed.

For an amino acid substitution, the following nomenclature is used herein: Original amino acid, position, substituted amino acid. Accordingly, the substitution of alanine at position 43 with valine is designated as “Ala43Val” or “A43V”. Multiple mutations are separated by forward slash marks (“/”), e.g., “N11D/S16G/N31S”, representing substitutions at positions 11, 16 and 31, respectively. The name, 3-letter abbreviation, and 1-letter abbreviation for each of the 20 amino acids is shown in Table 1.

TABLE 1
The name, 3-letter abbreviation, and 1-letter
abbreviation for each of the 20 amino acids.

| | 3-Letter | 1-Letter |
|---|---|---|
| Amino Acid | Code | Code |
| Alanine | Ala | A |
| Cysteine | Cys | C |
| Aspartic acid or aspartate | Asp | D |
| Glutamic acid or glutamate | Glu | E |
| Phenylalanine | Phe | F |
| Glycine | Gly | G |
| Histidine | His | H |
| Isoleucine | Ile | I |
| Lysine | Lys | K |
| Leucine | Leu | L |
| Methionine | Met | M |
| Asparagine | Asn | N |
| Proline | Pro | P |
| Glutamine | Gln | Q |
| Arginine | Arg | R |
| Serine | Ser | S |
| Threonine | Thr | T |
| Valine | Val | V |
| Tryptophan | Trp | W |
| Tyrosine | Tyr | Y |

The term “nucleic acid construct” refers to a nucleic acid molecule, either single-stranded or double-stranded, which is isolated from a naturally occurring gene or is modified to contain segments of nucleic acids in a manner that would not otherwise exist in nature or which is synthetic, and which comprises one or more control sequences.

The term “operably linked” refers to a configuration in which a control sequence is placed at an appropriate position relative to the coding sequence of a polynucleotide such that the control sequence directs expression of the coding sequence.

“Fc variant” or “variant Fc” as used herein is meant a protein comprising at least one amino acid sequence modification as compared to a parental Fc domain. In some embodiments, the parent Fc domain, is a human wild type Fc sequence, such as the Fc region from IgG1, IgG2, or IgG3. In some embodiments, the parent Fc domains are human Fc sequences with variants. For all positions discussed in the present invention that relate to the Fc domain of a human IgG, unless otherwise noted, amino acid position numbering is according to the EU index. The modification can be an addition, deletion, substitution or any combination thereof as outlined herein. Alternatively, the variant Fc domains can have from 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. Additionally, as discussed herein, the variant Fc domains herein still retain the ability to form a dimer with another Fc domain as well as bind to the FcRn receptor as measured using known techniques as described herein, such as non-denaturing gel electrophoresis.

The term “soluble BCMA” or “sBCMA” herein is meant a soluble portion of BCMA containing the extracellular domain (ECD) or a fragment or truncated version thereof, but not the entirety of the transmembrane domain or the cytoplasmic (intracellular) domain of BCMA. The ECD of human wild type sBCMA is shown as SEQ ID NO:1. In some embodiments, the parent wild type sBCMA domain can have N-terminal and/or C terminal truncations as long as the truncated wild type sBCMA retains biological activity, e.g. binding to APRIL and/or BAFF, as discussed below.

The term “sBCMA variant” or “variant sBCMA” refers to a variant of a parent sBCMA protein by virtue of at least one amino acid sequence modification. In some embodiments, the parent protein is a human wild type sBCMA. In some embodiments, the sBCMA variant retains specific binding to TGF family member(s), such as APRIL and/or BAFF, but has amino acid sequence modifications, e.g. amino acid substitutions, and can have N- or C-terminal truncations as compared to wild type sBCMA. Specific binding in this case is determined by any appropriate binding assay, such as ELISA, Biacore, Sapidyne KinExA, or Flow Cytometry binding analysis, which assays can also be used to determine binding affinity as outlined below. As discussed herein, sBCMA variants may have, in some instances, increased binding affinity for TGF family members (e.g. APRIL and/or BAFF) as compared to wild type sBCMA.

The term “binding affinity” refers to the ability of a ligand or variant thereof to form coordinated bonds with a protein, e.g., a receptor or a variant thereof. The binding affinity between a ligand and protein can be represented by an equilibrium dissociation constant (Kd), a ratio of koff/kon between the ligand and the protein (e.g., receptor or a variant thereof). Kd and binding affinity are inversely related. For instance, the Kd value relates the concentration of the sBCMA variant needed to bind to a TGF family member, and a lower Kd value (lower sBCMA variant concentration) corresponds to a higher binding affinity for the TGF family member. A high binding affinity corresponds to a greater intermolecular force between the ligand and the protein. A low binding affinity corresponds to a lower intermolecular force between the ligand and the protein. In some cases, an increase in ligand binding affinity can be represented as a decrease of the off-rate by, for example, at least 1.4-fold, at least 1.6-fold, at least 1.8-fold, at least 2-fold, at least 3-fold, at least 4-fold, at least 5-fold, at least 6-fold, at least 7-fold, at least 8-fold, at least 9-fold, at least 10-fold, at least 20-fold, at least 50-fold, at least 100-fold, at least 200-fold, at least 500-fold, or more.

“Specific binding” or “specifically binds to” or is “specific for” a particular ligand or variant thereof means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target. In some embodiments, the binding affinity is measured using any appropriate assay as would be understood by those skilled in the art as discussed above, such as a standard Biacore assay.

Specific binding for a particular ligand or variant thereof can be exhibited, for example, by a protein having a Kd for another ligand protein of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, at least about 10⁻¹⁵ M, or greater, where Kd refers to a dissociation rate of a particular protein-ligand interaction. In some embodiments, the variant sBCMA(s) of the present invention bind(s) a ligand with a binding affinity that is 1.5-, 2-, 3-, 4-, 5-, 6-, 7-, 8-, 9-, 10-, 15-, 20-, 50-, 100-, 200-, 500-, 1000-, 5,000-, 10,000- or more times greater as compared with a control molecule.

By “residue” as used herein is meant a position in a protein and its associated amino acid identity. For example, Asparagine 297 (also referred to as Asn297 or N297) is a residue at position 297 in the human antibody IgG1.

By “hinge” or “hinge region” or “antibody hinge region” or “hinge domain” herein is meant the flexible polypeptide comprising the amino acids between the first and second constant domains of an antibody. Structurally, the IgG CH1 domain ends at EU position 215, and the IgG CH2 domain begins at residue EU position 231. Thus for IgG, the antibody hinge is herein defined to include positions 216 (E216 in IgG1) to 230 (p230 in IgG1), wherein the numbering is according to the EU index as in Kabat. In some cases, a “hinge fragment” is used, which contains fewer amino acids at either or both of the N- and C-termini of the hinge domain. As outlined herein, in some cases, Fe domains inclusive of the hinge are used, with the hinge generally being used as a flexible linker. (Additionally, as further described herein, additional flexible linker components can be used either with or without the hinge).

By “Fc” or “Fc region” or “Fc domain” as used herein is meant the polypeptide comprising the CH2-CH3 domains of an IgG molecule, and in some cases, inclusive of the hinge. In EU numbering for human IgG1, the CH2-CH3 domain comprises amino acids 231 to 447, and the hinge is 216 to 230. Thus the definition of “Fc domain” includes both amino acids 231-447 (CH2-CH3) or 216-447 (hinge-CH2-CH3), or fragments thereof. Thus Fc refers to the last two constant region immunoglobulin domains of IgA, IgD, and IgG, the last three constant region immunoglobulin domains of IgE and IgM, and in some cases, includes the flexible hinge N-terminal to these domains. For IgA and IgM, Fc may include the J chain. For IgG, the Fc domain comprises immunoglobulin domains Cγ2 and Cγ3 and in some cases, includes the lower hinge region between Cγ1 and Cγ2. An “Fc fragment” in this context may contain fewer amino acids from either or both of the N- and C-termini but still retains the ability to form a dimer with another Fc domain or Fc fragment as can be detected using standard methods, generally based on size (e.g. non-denaturing chromatography, size exclusion chromatography, etc). Human IgG Fc domains are of particular use in the present invention, and can be the Fc domain from human IgG1, IgG2, or IgG3. In general, IgG1 and IgG2 are used more frequently than IgG3. In some embodiments, amino acid sequence modifications are made to the Fc region, for example to alter binding to one or more FcγR receptors or to the FcRn receptor, and/or to increase the half-life in vivo.

By “IgG subclass modification” or “isotype modification” as used herein is meant an amino acid sequence modification that exchanges one amino acid of one IgG isotype to the corresponding amino acid in a different, aligned IgG isotype. For example, because IgG1 comprises a tyrosine and IgG2 comprises a phenylalanine at EU position 296, a F296Y substitution in IgG2 is considered an IgG subclass modification. Similarly, because IgG1 has a proline at position 241 and IgG4 has a serine, an IgG4 molecule with a S241P is considered an IgG subclass modification. Note that subclass modifications are considered amino acid substitutions herein.

By “amino acid” and “amino acid identity” as used herein is meant one of the 20 naturally occurring amino acids that are coded for by DNA and RNA.

By “effector function” as used herein is meant a biochemical event that results from the interaction of an antibody Fc region with an Fc receptor or ligand. Effector functions include but are not limited to antibody-dependent cellular cytotoxicity (ADCC), antibody-dependent cellular phagocytosis (ADCP), and complement-dependent cytotoxicity (CDC). In many cases, it is desirable to ablate most or all effector functions using either different IgG isotypes (e.g. IgG4) or amino acid substitutions in the Fc domain; however, preserving binding to the FcRn receptor is desirable, as this contributes to the half-life of the fusion protein in human serum.

By “FcRn” or “neonatal Fc Receptor” as used herein is meant a protein that binds the IgG antibody Fc region and is encoded at least in part by an FcRn gene.

By “target cell” as used herein is meant a cell that expresses a target polypeptide or protein.

By “host cell” in the context of producing the variant sBCMA or the sBCMA variant—Fc fusion proteins according to the invention herein is meant a cell that contains the exogenous nucleic acids encoding the components of the variant sBCMA or the sBCMA variant—Fc fusion protein, and is capable of expressing such variant sBCMA or Fc fusion protein under suitable conditions. Suitable host cells are described below.

By “improved activity” or “improved function” herein meant a desirable change of at least one biochemical property. An improved function in this context can be measured as a percentage increase or decrease of a particular activity, or as a “fold” change, with increases of desirable properties (e.g. increased binding affinity and/or specificity for APRIL and/or BAFF, increased protein stability of the, increased half-life in vivo, etc.). In general, percentage changes are used to describe changes in biochemical activity of less than 100%, and fold-changes are used to describe changes in biochemical activity of greater than 100% (as compared to the parent protein). In the present invention, percentage changes (usually increases) of biochemical activity of at least about 10%, 20%, 30%, 40%, 50%, 60%, 70%, 80%, 90%, 95%, 98% and 99% can be accomplished. In the present invention, a “fold increase” (or decrease) is measured as compared to the parent protein. In many embodiments, the improvement is at least 1.4 fold, 1.5 fold, 1.6 fold, 1.8 fold, 2 fold, 3 fold, 4 fold, 5 fold, 6 fold, 7 fold, 8 fold, 9 fold, 10 fold, 50 fold, 100 fold, 200 fold or higher.

### C. sBCMA Variant—Fc Fusion Proteins

The sBCMA variant—Fc fusion proteins of the present invention include a composition comprising a variant sBCMA domain, an Fc domain, and optionally a linker linking the variant sBCMA domain with the Fc domain.

In some embodiments, the present invention provides the composition as described herein, wherein said fusion protein comprises, from N- to C-terminal:
a) said variant sBCMA domain;
b) said optional linker; and
c) said Fc domain.

In some embodiments, the present invention provides the composition as described herein, wherein said fusion protein comprises, from N- to C-terminal:
a) said Fc domain;
b) said optional linker; and
c) said variant sBCMA domain.

### D. Variant sBCMA Proteins and Domains

The invention provides variant sBCMA proteins both independently and as fusion protein constructs as an sBCMA domain fused with Fe domains. Variant sBCMA proteins of the present invention include at least a portion of the soluble ECD of human BCMA, generally the entire ECD domain (SEQ ID NO:1) as shown in FIG. 7, with amino acid variants. In some embodiments, variant sBCMA proteins or sBCMA variants exhibits increased binding affinity and/or specificity for APRIL and/or BAFF as compared to wild-type sBCMA as determined by binding affinity assays in the art and discussed below, such as Biacore or Octet assays.

In some embodiments, variant sBCMA proteins (either as isolated proteins or as sBCMA domains of the fusion proteins herein) are antagonists that bind to APRIL and/or BAFF to mitigate or to block their interaction with endogenous BCMA, BAFFR, and TACI receptors. Variant sBCMA proteins as antagonists can be used in treating conditions associated with altered signaling pathways through BCMA, BAFFR, TACI and/or other receptors that are activated through binding to APRIL and/or BAFF, in particular tumor therapy/chemotherapy, immunomodulatory and/or fibrotic diseases.

In one embodiment, variant sBCMA proteins (either as isolated proteins or as sBCMA domains of the fusion proteins herein) can be used in a method of treating, reducing or preventing metastasis or invasion of a tumor that expresses APRIL in a subject, said method comprising administering to the subject a therapeutically effective dose of one or more said variant sBCMA proteins as disclosed herein.

In one embodiment, variant sBCMA proteins (either as isolated proteins or as sBCMA domains of the fusion proteins herein) can be used in a method of treating, reducing or preventing metastasis or invasion of a tumor that expresses BCMA, TACI and/or other receptors that are activated through binding to APRIL in a subject, said method comprising administering to the subject a therapeutically effective dose of one or more said variant sBCMA proteins as disclosed herein. In some embodiments, the tumor as disclosed herein is a B-cell malignant disease. In some embodiments, the B-cell malignant disease as disclosed herein is selected from the group consisting of multiple myeloma, diffuse large B-cell lymphoma, primary mediastinal B-cell lymphoma, follicular lymphoma, chronic lymphocytic leukemia, mantle cell lymphoma, extranodal marginal zone B-cell lymphoma—mucosa-associated lymphoid tissue (MALT) lymphoma, nodal marginal zone B-cell lymphoma, splenic marginal zone B-cell lymphoma, Burkitt lymphoma, lymphoplasmacytic lymphoma (Waldenstrom macroglobulinemia), hairy cell leukemia, primary central nervous system (CNS) lymphoma, and primary intraocular lymphoma (lymphoma of the eye). In some embodiments, the tumor as disclosed herein is a hematologic cancer. In some embodiments, the hematologic cancer as disclosed herein is multiple myeloma.

In a further embodiment, variant sBCMA proteins (either as isolated proteins or as sBCMA domains of the fusion proteins herein) can be used in a method of inhibiting the activity of APRIL in a subject having a tumor that expresses APRIL, said method comprising administering to the subject a therapeutically effective dose of one or more said variant sBCMA proteins as disclosed herein. In some embodiments, the tumor as disclosed herein is a B-cell malignant disease. In some embodiments, the B-cell malignant disease as disclosed herein is selected from the group consisting of multiple myeloma, diffuse large B-cell lymphoma, primary mediastinal B-cell lymphoma, follicular lymphoma, chronic lymphocytic leukemia, mantle cell lymphoma, extranodal marginal zone B-cell lymphoma—mucosa-associated lymphoid tissue (MALT) lymphoma, nodal marginal zone B-cell lymphoma, splenic marginal zone B-cell lymphoma, Burkitt lymphoma, lymphoplasmacytic lymphoma (Waldenstrom macroglobulinemia), hairy cell leukemia, primary central nervous system (CNS) lymphoma, and primary intraocular lymphoma (lymphoma of the eye). In some embodiments, the tumor as disclosed herein is a hematologic cancer. In some embodiments, the hematologic cancer as disclosed herein is multiple myeloma.

In a further embodiment, variant sBCMA proteins (either as isolated proteins or as sBCMA domains of the fusion proteins herein) can be used in a method of inhibiting the activity of APRIL in a subject having a tumor that expresses BCMA, TACI and/or other receptors that are activated through binding to APRIL, said method comprising administering to the subject a therapeutically effective dose of one or more said variant sBCMA proteins as disclosed herein. In some embodiments, the tumor as disclosed herein is a B-cell malignant disease. In some embodiments, the B-cell malignant disease as disclosed herein is selected from the group consisting of multiple myeloma, diffuse large B-cell lymphoma, primary mediastinal B-cell lymphoma, follicular lymphoma, chronic lymphocytic leukemia, mantle cell lymphoma, extranodal marginal zone B-cell lymphoma—mucosa-associated lymphoid tissue (MALT) lymphoma, nodal marginal zone B-cell lymphoma, splenic marginal zone B-cell lymphoma, Burkitt lymphoma, lymphoplasmacytic lymphoma (Waldenstrom macroglobulinemia), hairy cell leukemia, primary central nervous system (CNS) lymphoma, and primary intraocular lymphoma (lymphoma of the eye). In some embodiments, the tumor as disclosed herein is a hematologic cancer. In some embodiments, the hematologic cancer as disclosed herein is multiple myeloma.

In an additional embodiment, variant sBCMA proteins (either as isolated proteins or as sBCMA domains of the fusion proteins herein) can be used in a method of inhibiting the activity of APRIL in a subject having an autoimmune disease that expresses APRIL, said method comprising administering to the subject a therapeutically effective dose of one or more said variant sBCMA proteins as disclosed herein.

In an additional embodiment, variant sBCMA proteins (either as isolated proteins or as sBCMA domains of the fusion proteins herein) can be used in a method of inhibiting the activity of APRIL in a subject having an autoimmune disease that expresses BCMA, TACI and/or other receptors that are activated through binding to APRIL, said method comprising administering to the subject a therapeutically effective dose of one or more said variant sBCMA proteins as disclosed herein.

In a further embodiment, variant sBCMA proteins (either as isolated proteins or as sBCMA domains of the fusion proteins herein) can be used in a method of inhibiting the activity of APRIL in a subject having fibrosis that expresses APRIL, said method comprising administering to the subject a therapeutically effective dose of one or more said variant sBCMA proteins as disclosed herein.

In a further embodiment, variant sBCMA proteins (either as isolated proteins or as sBCMA domains of the fusion proteins herein) can be used in a method of inhibiting the activity of APRIL in a subject having fibrosis that expresses BCMA, TACI and/or other receptors that are activated through binding to APRIL, said method comprising administering to the subject a therapeutically effective dose of one or more said variant sBCMA proteins as disclosed herein.

In an additional embodiment, variant sBCMA proteins (either as isolated proteins or as sBCMA domains of the fusion proteins herein) can be used in a method of inhibiting B-cell growth, immunoglobulin production, or both in a subject, where the variant sBCMA protein binds to BAFF, said method comprising administering to the subject a therapeutically effective dose of one or more said variant sBCMA proteins as disclosed herein.

In a further embodiment, variant sBCMA proteins (either as isolated proteins or as sBCMA domains of the fusion proteins herein) can be used in a method of inhibiting the activity of BAFF in a subject having B cell hyperplasia or an autoimmune disease expressing BCMA, BAFFR, TACI and/or other receptors that are activated through binding to BAFF, said method comprising administering to the subject a therapeutically effective dose of one or more said variant sBCMA proteins as disclosed herein.

In an additional embodiment, variant sBCMA proteins (either as isolated proteins or as sBCMA domains of the fusion proteins herein) can be used in a method of treating an autoimmune disease expressing at least one receptor selected from the group consisting of BCMA, BAFFR, TACI and other receptor(s) that are activated through binding to BAFF in a subject, said method comprising administering to the subject a therapeutically effective dose of one or more said variant sBCMA proteins as disclosed herein.

In a further embodiment, variant sBCMA proteins (either as isolated proteins or as sBCMA domains of the fusion proteins herein) can be used in a method of treating an autoimmune disease expressing BAFF and/or APRIL in a subject, said method comprising administering to the subject a therapeutically effective dose of one or more said variant sBCMA proteins as disclosed herein.

In an additional embodiment, variant sBCMA proteins (either as isolated proteins or as sBCMA domains of the fusion proteins herein) can be used in a method of treating fibrosis expressing BCMA, BAFFR and/or TACI in a subject, said method comprising administering to the subject a therapeutically effective dose of one or more said variant sBCMA proteins as disclosed herein.

In an additional embodiment, variant sBCMA proteins (either as isolated proteins or as sBCMA domains of the fusion proteins herein) can be used in a method of treating fibrosis expressing BAFF and/or APRIL in a subject, said method comprising administering to the subject a therapeutically effective dose of one or more said variant sBCMA proteins as disclosed herein.

In some embodiments, variant sBCMA proteins include amino acid substitutions, deletions or insertions or any combination thereof as compared to the wild type sBCMA, and increase their binding activity to either APRIL, BAFF or both as compared to the wild-type sBCMA.

The present disclosure provides variant sBCMA protein(s) comprising at least one amino acid substitution at one or more (e.g., 2, 3, 4, 5, 6, 7, 8, 9 or 10) positions as compared to a parent sBCMA. In some embodiments, a variant sBCMA has at least 80%, at least 85%, at least 90%, at least 95%, at least 96%, at least 97%, at least 98%, or at least 99%, but less than 100% sequence identity to the parent sBCMA. In some embodiments, a parent sBCMA domain is human wild-type sBCMA. In some embodiments, a parent sBCMA domain has the amino acid sequence of SEQ ID NO:1. In some embodiments, a variant sBCMA has at least 90%, at least 91%, at least 92%, at least 93%, at least 94%, at least 95%, at least 96%, at least 97%, at least 98%, or at least 99%, but less than 100% sequence identity to SEQ ID NO:1. In some embodiments, as noted herein, a variant sBCMA can have N-terminal and/or C terminal truncations compared to wild type sBCMA as long as the truncated variant sBCMA retains biological activity (e.g. binding to APRIL and/or BAFF), as measured by one of the binding assays outlined herein. To be clear, the variant BCMA of the present invention has at least one amino acid substitution as compared to SEQ ID NO:1, and thus is not SEQ ID NO:1.

In some embodiments, a variant sBCMA described herein has a binding affinity for TGF family member (i.e., APRIL and/or BAFF) that is stronger than the wild-type sBCMA polypeptide/domain. In some embodiments, the variant sBCMA has a binding affinity for APRIL and/or BAFF that is at least 1.4-fold, 1.5-fold, 1.6-fold, 1.8-fold, 2-fold, 3-fold, 4-fold, 5-fold, 10-fold, 50-fold, 100-fold, 200-fold or greater than that of the wild-type sBCMA.

In certain embodiments, the binding affinity of the variant sBCMA for APRIL and/or BAFF is increased by at least about 0.5%, 1%, 2%, 3%, 4%, 5%, 6%, 7%, 8%, 9%, 10%, 15%, 20%, 25%, 30%, 35%, 40%, 45%, 50% or higher as compared to that of the wild-type sBCMA. In other embodiments, the variant sBCMA proteins of the present invention have a Kd value of less than about 1×10⁻⁸ M, 1×10⁻⁹ M, 1×10⁻¹⁰ M, 1×10⁻¹² M or 1×10⁻¹⁵ M for binding with APRIL and/or BAFF. In yet other embodiments, sBCMA variants inhibit or compete with wild-type sBCMA in binding to APRIL and/or BAFF either in vivo, in vitro or both.

### I. Specific Variant sBCMA Proteins

The present invention provides a composition comprising a variant sBCMA comprising at least one amino acid substitution as compared to SEQ ID NO:1, wherein said amino acid substitution is at a position number selected from the group consisting of 1, 2, 3, 4, 5, 6, 7, 9, 10, 11, 12, 14, 16, 19, 20, 22, 23, 25, 26, 29, 31, 32, 35, 36, 38, 39, 42, 43, 44, 45, 46, 47, 48, 49, 50, 51, 52, 53, and 54, wherein the numbering is according to the EU index.

In some embodiments, the variant sBCMA as described herein has at least 80%, at least 85%, at least 90%, or at least 95% sequence identity to SEQ ID NO:1.

In some embodiments, the variant sBCMA as described herein comprises an amino acid substitution of the methionine at position 1 with the position numbering starting from the mature region. In some embodiments, the substitution is with any other of the 19 naturally occurring amino acids, serine, threonine, asparagine, glutamic acid, glutamine, aspartic acid, lysine, arginine, histidine, cysteine, glycine, alanine, isoleucine, leucine, proline, phenylalanine, tryptophan, valine and tyrosine, with some embodiments not utilizing proline (due to steric effects). In some embodiments, the amino acid substitution is selected from M1A, M1C, M1I, M1R, M1T, and M1V.

In some embodiments, the variant sBCMA as described herein comprises an amino acid substitution of the leucine at position 2 with the position numbering starting from the mature region. In some embodiments, the substitution is with any other of the 19 naturally occurring amino acids, serine, threonine, asparagine, glutamic acid, glutamine, aspartic acid, lysine, arginine, histidine, cysteine, glycine, alanine, isoleucine, methionine, proline, phenylalanine, tryptophan, valine and tyrosine, with some embodiments not utilizing proline (due to steric effects). In some embodiments, the amino acid substitution is L2C or L2S.

In some embodiments, the variant sBCMA as described herein comprises an amino acid substitution of the glutamine at position 3 with the position numbering starting from the mature region. In some embodiments, the substitution is with any other of the 19 naturally occurring amino acids, serine, threonine, asparagine, glutamic acid, aspartic acid, lysine, arginine, histidine, cysteine, glycine, alanine, isoleucine, leucine, methionine, proline, phenylalanine, tryptophan, valine and tyrosine, with some embodiments not utilizing cysteine (due to possible disulfide formation). In some embodiments, the amino acid substitution is Q3P or Q3R.

In some embodiments, the variant sBCMA as described herein comprises an amino acid substitution of the methionine at position 4 with the position numbering starting from the mature region. In some embodiments, the substitution is with any other of the 19 naturally occurring amino acids, serine, threonine, asparagine, glutamic acid, glutamine, aspartic acid, lysine, arginine, histidine, cysteine, glycine, alanine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is selected from M4E, M4I, M4T, and M4V.

In some embodiments, the variant sBCMA as described herein comprises an amino acid substitution of the alanine at position 5 with the position numbering starting from the mature region. In some embodiments, the substitution is with any other of the 19 naturally occurring amino acids, serine, threonine, asparagine, glutamic acid, glutamine, aspartic acid, lysine, arginine, histidine, cysteine, glycine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is A5T.

In some embodiments, the variant sBCMA as described herein comprises an amino acid substitution of the glycine at position 6 with the position numbering starting from the mature region. In some embodiments, the substitution is with any other of the 19 naturally occurring amino acids, serine, threonine, asparagine, glutamic acid, glutamine, aspartic acid, lysine, arginine, histidine, cysteine, alanine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is G6E.

In some embodiments, the variant sBCMA as described herein comprises an amino acid substitution of the glutamine at position 7 with the position numbering starting from the mature region. In some embodiments, the substitution is with any other of the 19 naturally occurring amino acids, serine, threonine, asparagine, glutamic acid, aspartic acid, lysine, arginine, histidine, cysteine, glycine, alanine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is Q7R.

In some embodiments, the variant sBCMA as described herein comprises an amino acid substitution of the serine at position 9 with the position numbering starting from the mature region. In some embodiments, the substitution is with any other of the 19 naturally occurring amino acids, threonine, asparagine, glutamic acid, glutamine, aspartic acid, lysine, arginine, histidine, cysteine, glycine, alanine, isoleucine, leucine, methionine, proline, phenylalanine, tryptophan, valine and tyrosine, with some embodiments not utilizing cysteine (due to possible disulfide formation). In some embodiments, the amino acid substitution is selected from S9A, S9F and S9P.

In some embodiments, the variant sBCMA as described herein comprises an amino acid substitution of the glutamine at position 10 with the position numbering starting from the mature region. In some embodiments, the substitution is with any other of the 19 naturally occurring amino acids, serine, threonine, asparagine, glutamic acid, aspartic acid, lysine, arginine, histidine, cysteine, glycine, alanine, isoleucine, leucine, methionine, proline, phenylalanine, tryptophan, valine and tyrosine, with some embodiments not utilizing cysteine (due to possible disulfide formation). In some embodiments, the amino acid substitution is selected from Q10H, Q10P and Q10R.

In some embodiments, the variant sBCMA as described herein comprises an amino acid substitution of the asparagine at position 11 with the position numbering starting from the mature region. In some embodiments, the substitution is with any other of the 19 naturally occurring amino acids, serine, threonine, glutamic acid, glutamine, aspartic acid, lysine, arginine, histidine, cysteine, glycine, alanine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is Ni 1D or N11S.

In some embodiments, the variant sBCMA as described herein comprises an amino acid substitution of the glutamic acid at position 12 with the position numbering starting from the mature region. In some embodiments, the substitution is with any other of the 19 naturally occurring amino acids, serine, threonine, asparagine, glutamine, aspartic acid, lysine, arginine, histidine, cysteine, glycine, alanine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is E12K.

In some embodiments, the variant sBCMA as described herein comprises an amino acid substitution of the phenylalanine at position 14 with the position numbering starting from the mature region. In some embodiments, the substitution is with any other of the 19 naturally occurring amino acids, serine, threonine, asparagine, glutamic acid, glutamine, aspartic acid, lysine, arginine, histidine, cysteine, glycine, alanine, isoleucine, leucine, methionine, proline, tryptophan, valine and tyrosine, with some embodiments not utilizing cysteine (due to possible disulfide formation) or proline (due to steric effects). In some embodiments, the amino acid substitution is F14L.

In some embodiments, the variant sBCMA as described herein comprises an amino acid substitution of the serine at position 16 with the position numbering starting from the mature region. In some embodiments, the substitution is with any other of the 19 naturally occurring amino acids, threonine, asparagine, glutamic acid, glutamine, aspartic acid, lysine, arginine, histidine, cysteine, glycine, alanine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is selected from S16G, S16N, and S16R.

In some embodiments, the variant sBCMA as described herein comprises an amino acid substitution of the histidine at position 19 with the position numbering starting from the mature region. In some embodiments, the substitution is with any other of the 19 naturally occurring amino acids, serine, threonine, asparagine, glutamic acid, glutamine, aspartic acid, lysine, arginine, cysteine, glycine, alanine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is H19L or H19Y.

In some embodiments, the variant sBCMA as described herein comprises an amino acid substitution of the alanine at position 20 with the position numbering starting from the mature region. In some embodiments, the substitution is with any other of the 19 naturally occurring amino acids, serine, threonine, asparagine, glutamic acid, glutamine, aspartic acid, lysine, arginine, histidine, cysteine, glycine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is A20V or A20T.

In some embodiments, the variant sBCMA as described herein comprises an amino acid substitution of the isoleucine at position 22 with the position numbering starting from the mature region. In some embodiments, the substitution is with any other of the 19 naturally occurring amino acids, serine, threonine, asparagine, glutamic acid, glutamine, aspartic acid, lysine, arginine, histidine, cysteine, glycine, alanine, leucine, methionine, proline, phenylalanine, tryptophan, valine and tyrosine, with some embodiments not utilizing cysteine (due to possible disulfide formation) or proline (due to steric effects). In some embodiments, the amino acid substitution is I22M or I22V.

In some embodiments, the variant sBCMA as described herein comprises an amino acid substitution of the proline at position 23 with the position numbering starting from the mature region. In some embodiments, the substitution is with any other of the 19 naturally occurring amino acids, serine, threonine, asparagine, glutamic acid, glutamine, aspartic acid, lysine, arginine, histidine, cysteine, glycine, alanine, isoleucine, leucine, methionine, phenylalanine, tryptophan, valine and tyrosine, with some embodiments not utilizing cysteine (due to possible disulfide formation) or proline (due to steric effects). In some embodiments, the amino acid substitution is P23S.

In some embodiments, the variant sBCMA as described herein comprises an amino acid substitution of the glutamine at position 25 with the position numbering starting from the mature region. In some embodiments, the substitution is with any other of the 19 naturally occurring amino acids, serine, threonine, asparagine, glutamic acid, aspartic acid, lysine, arginine, histidine, cysteine, glycine, alanine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is Q25R.

In some embodiments, the variant sBCMA as described herein comprises an amino acid substitution of the leucine at position 26 with the position numbering starting from the mature region. In some embodiments, the substitution is with any other of the 19 naturally occurring amino acids, serine, threonine, asparagine, glutamic acid, glutamine, aspartic acid, lysine, arginine, histidine, cysteine, glycine, alanine, isoleucine, methionine, proline, phenylalanine, tryptophan, valine and tyrosine, with some embodiments not utilizing cysteine (due to possible disulfide formation) or proline (due to steric effects). In some embodiments, the amino acid substitution is L26F.

In some embodiments, the variant sBCMA as described herein comprises an amino acid substitution of the serine at position 29 with the position numbering starting from the mature region. In some embodiments, the substitution is with any other of the 19 naturally occurring amino acids, threonine, asparagine, glutamic acid, glutamine, aspartic acid, lysine, arginine, histidine, cysteine, glycine, alanine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is S29A.

In some embodiments, the variant sBCMA as described herein comprises an amino acid substitution of the asparagine at position 31 with the position numbering starting from the mature region. In some embodiments, the substitution is with any other of the 19 naturally occurring amino acids, serine, threonine, glutamic acid, glutamine, aspartic acid, lysine, arginine, histidine, cysteine, glycine, alanine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is N31D or N31S.

In some embodiments, the variant sBCMA as described herein comprises an amino acid substitution of the threonine at position 32 with the position numbering starting from the mature region. In some embodiments, the substitution is with any other of the 19 naturally occurring amino acids, serine, asparagine, glutamic acid, glutamine, aspartic acid, lysine, arginine, histidine, cysteine, glycine, alanine, isoleucine, leucine, methionine, proline, phenylalanine, tryptophan, valine and tyrosine, with some embodiments not utilizing cysteine (due to possible disulfide formation). In some embodiments, the amino acid substitution is selected from T32A, T32I and T32P.

In some embodiments, the variant sBCMA as described herein comprises an amino acid substitution of the leucine at position 35 with the position numbering starting from the mature region. In some embodiments, the substitution is with any other of the 19 naturally occurring amino acids, serine, threonine, asparagine, glutamic acid, glutamine, aspartic acid, lysine, arginine, histidine, cysteine, glycine, alanine, isoleucine, methionine, proline, phenylalanine, tryptophan, valine and tyrosine, with some embodiments not utilizing cysteine (due to possible disulfide formation). In some embodiments, the amino acid substitution is L35S or L35P.

In some embodiments, the variant sBCMA as described herein comprises an amino acid substitution of the threonine at position 36 with the position numbering starting from the mature region. In some embodiments, the substitution is with any other of the 19 naturally occurring amino acids, serine, asparagine, glutamic acid, glutamine, aspartic acid, lysine, arginine, histidine, cysteine, glycine, alanine, isoleucine, leucine, methionine, proline, phenylalanine, tryptophan, valine and tyrosine, with some embodiments not utilizing cysteine (due to possible disulfide formation). In some embodiments, the amino acid substitution is selected from T36A, T36I, and T36P.

In some embodiments, the variant sBCMA as described herein comprises an amino acid substitution of the glutamine at position 38 with the position numbering starting from the mature region. In some embodiments, the substitution is with any other of the 19 naturally occurring amino acids, serine, threonine, asparagine, glutamic acid, aspartic acid, lysine, arginine, histidine, cysteine, glycine, alanine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is Q38R.

In some embodiments, the variant sBCMA as described herein comprises an amino acid substitution of the arginine at position 39 with the position numbering starting from the mature region. In some embodiments, the substitution is with any other of the 19 naturally occurring amino acids, serine, threonine, asparagine, glutamic acid, glutamine, aspartic acid, lysine, histidine, cysteine, glycine, alanine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is R39H.

In some embodiments, the variant sBCMA as described herein comprises an amino acid substitution of the asparagine at position 42 with the position numbering starting from the mature region. In some embodiments, the substitution is with any other of the 19 naturally occurring amino acids, serine, threonine, glutamic acid, glutamine, aspartic acid, lysine, arginine, histidine, cysteine, glycine, alanine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is selected from N42D, N42R and N42S.

In some embodiments, the variant sBCMA as described herein comprises an amino acid substitution of the alanine at position 43 with the position numbering starting from the mature region. In some embodiments, the substitution is with any other of the 19 naturally occurring amino acids, serine, threonine, asparagine, glutamic acid, glutamine, aspartic acid, lysine, arginine, histidine, cysteine, glycine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is A43T or A43V.

In some embodiments, the variant sBCMA as described herein comprises an amino acid substitution of the serine at position 44 with the position numbering starting from the mature region. In some embodiments, the substitution is with any other of the 19 naturally occurring amino acids, threonine, asparagine, glutamic acid, glutamine, aspartic acid, lysine, arginine, histidine, cysteine, glycine, alanine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is selected from S44D, S44G, S44N and S44R.

In some embodiments, the variant sBCMA as described herein comprises an amino acid substitution of the valine at position 45 with the position numbering starting from the mature region. In some embodiments, the substitution is with any other of the 19 naturally occurring amino acids, serine, threonine, asparagine, glutamic acid, glutamine, aspartic acid, lysine, arginine, histidine, cysteine, glycine, alanine, isoleucine, leucine, methionine, proline, phenylalanine, tryptophan and tyrosine, with some embodiments not utilizing cysteine (due to possible disulfide formation) or proline (due to steric effects). In some embodiments, the amino acid substitution is V45A or V45M.

In some embodiments, the variant sBCMA as described herein comprises an amino acid substitution of the threonine at position 46 with the position numbering starting from the mature region. In some embodiments, the substitution is with any other of the 19 naturally occurring amino acids, serine, asparagine, glutamic acid, glutamine, aspartic acid, lysine, arginine, histidine, cysteine, glycine, alanine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is T46A or T46I.

In some embodiments, the variant sBCMA as described herein comprises an amino acid substitution of the asparagine at position 47 with the position numbering starting from the mature region. In some embodiments, the substitution is with any other of the 19 naturally occurring amino acids, serine, threonine, glutamic acid, glutamine, aspartic acid, lysine, arginine, histidine, cysteine, glycine, alanine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is selected from N47D, N47K, N47R and N47S.

In some embodiments, the variant sBCMA as described herein comprises an amino acid substitution of the serine at position 48 with the position numbering starting from the mature region. In some embodiments, the substitution is with any other of the 19 naturally occurring amino acids, threonine, asparagine, glutamic acid, glutamine, aspartic acid, lysine, arginine, histidine, cysteine, glycine, alanine, isoleucine, leucine, methionine, proline, phenylalanine, tryptophan, valine and tyrosine, with some embodiments not utilizing cysteine (due to possible disulfide formation). In some embodiments, the amino acid substitution is selected from S48L, S48P and S48T.

In some embodiments, the variant sBCMA as described herein comprises an amino acid substitution of the valine at position 49 with the position numbering starting from the mature region. In some embodiments, the substitution is with any other of the 19 naturally occurring amino acids, serine, threonine, asparagine, glutamic acid, glutamine, aspartic acid, lysine, arginine, histidine, cysteine, glycine, alanine, isoleucine, leucine, methionine, proline, phenylalanine, tryptophan, and tyrosine, with some embodiments not utilizing cysteine (due to possible disulfide formation) or proline (due to steric effects). In some embodiments, the amino acid substitution is V49A or V49M.

In some embodiments, the variant sBCMA as described herein comprises an amino acid substitution of the lysine at position 50 with the position numbering starting from the mature region. In some embodiments, the substitution is with any other of the 19 naturally occurring amino acids, serine, threonine, asparagine, glutamic acid, glutamine, aspartic acid, arginine, histidine, cysteine, glycine, alanine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is selected from K50E, K50G, K50R and K50T.

In some embodiments, the variant sBCMA as described herein comprises an amino acid substitution of the glycine at position 51 with the position numbering starting from the mature region. In some embodiments, the substitution is with any other of the 19 naturally occurring amino acids, serine, threonine, asparagine, glutamic acid, glutamine, aspartic acid, lysine, arginine, histidine, cysteine, alanine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is G51E.

In some embodiments, the variant sBCMA as described herein comprises an amino acid substitution of the threonine at position 52 with the position numbering starting from the mature region. In some embodiments, the substitution is with any other of the 19 naturally occurring amino acids, serine, asparagine, glutamic acid, glutamine, aspartic acid, lysine, arginine, histidine, cysteine, glycine, alanine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is T52A or T52M.

In some embodiments, the variant sBCMA as described herein comprises an amino acid substitution of the asparagine at position 53 with the position numbering starting from the mature region. In some embodiments, the substitution is with any other of the 19 naturally occurring amino acids, serine, threonine, glutamic acid, glutamine, aspartic acid, lysine, arginine, histidine, cysteine, glycine, alanine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is selected from N53D, N53K and N53S.

In some embodiments, the variant sBCMA as described herein comprises an amino acid substitution of the alanine at position 54 with the position numbering starting from the mature region. In some embodiments, the substitution is with any other of the 19 naturally occurring amino acids, serine, threonine, asparagine, glutamic acid, glutamine, aspartic acid, lysine, arginine, histidine, cysteine, glycine, isoleucine, leucine, methionine, proline, phenylalanine, tryptophan, valine and tyrosine, with some embodiments not utilizing cysteine (due to possible disulfide formation) or proline (due to steric effects). In some embodiments, the amino acid substitution is A54V or A54T.

In some embodiments, the variant sBCMA as described herein comprises amino acid substitution(s) selected from the group consisting of M1A, M1C, M1I, M1R, M1T, M1V, L2C, L2S, Q3P, Q3R, M4E, M4I, M4T, M4V, A5T, G6E, Q7R, S9A, S9F, S9P, Q10H, Q10P, Q10R, N11D, N11S, E12K, F14L, S16G, S16N, S16R, H19L, H19Y, A20V, A20T, I22M, I22V, P23S, Q25R, L26F, S29A, N31D, N31S, T32A, T32I, T32P, L35S, L35P, T36A, T36I, T36P, Q38R, R39H, N42D, N42R, N42S, A43T, A43V, S44D, S44G, S44N, S44R, V45A, V45M, T46A, T46I, N47D, N47K, N47R, N47S, S48L, S48P, S48T, V49A, V49M, K50E, K50G, K50R, K50T, G51E, T52A, T52M, N53D, N53K, N53S, A54V, and A54T.

In some embodiments, the variant sBCMA as described herein comprises amino acid substitution(s) selected from the group consisting of M1V, L2S, Q3P, M4T, S9P, N11D, S16G, H19Y, N31S, N31D, T32I, T36A, R39H, N47S, K50E, and N53E.

In some embodiments, the variant sBCMA as described herein comprises amino acid substitution(s) selected from the group consisting of S16G, H19Y and T36A.

In some embodiments, the variant sBCMA as described herein comprises amino acid substitutions selected from the group consisting of L2S/S9P/E12K/N31D/T36A/N42S/N53S, M1V/T32P/T36A/T46I/N53D/A54V, Q3R/S16N/T36A/A43T, F14L/S16G/T36A/V45A/N47D, M1T/M4V/S9F/S16G/T32A/Q38R, M1A/S9A/Q38R, G6E/Q25R/Q38R, M1V/M4I/G6E/S9P/N11D/V49M/T52M/A54V, N11D/S16G/N31S, N11D/H9Y/I22M/T32P/N47S/N53S, G6E/Q7R/H19Y/L35S, H19Y/N42D/S48P/T52A, M1V/N31D/T32I/T36A, M1V/A5T/H19L/T36A, M1T/N31D/T32A/T36A/Q38R/S44D/V49A/K50E, M1V/T36A/Q38R/A43V, M1V/L2S/S9P/Q10H/T36A/Q38R/K50G, T36A/Q38R/N53S, M1T/L2S/L35P/T36A/Q38R/T46A/K50R, A5T/A20V/T36A/Q38R, M1T/S16G/I22V/T36A/S44G/T46A/V49A, S16G/T36A, M1I/N11D/S16G/I22M/S29A/T36A/S44G/K50R, M1C/L2C/Q3R/M4E/N11D/S16G/T36P, M1I/N11D/S16G/I22M/S29A/T36A/S44G/K50R, N11D/N31D/T32I/T36A/S44N/N47D/N53D, M1R/L2C/Q3R, H19Y/T36A/S44G, H19Y/T32I/T36A/V49A, H19Y/N31S/T36A/V45A, H19Y/N31S/T36A, H19Y/T36P/T52A, H19Y/N31D/T52M, M1V/H19Y/V45M, S16G/H19Y/N47D, S16G/H19Y/K50T, S16G/H19Y/S44N/K50R, N11D/H19Y/S48T, S9P/N11D/S6R/T32A/Q38R/S44G/T46I/T52A/N53D/A54T, N11D/S16G/S44R, H19L/T32A/S44G/G51E/T52A, S16N/H19Y/T36A/K50R, M1V/H19Y/T36A/R39H/T46A, M1V/H19Y/T36A, H19Y/T36A/N42D/N47S/S48P, M1V/H19Y/T36A/S44G/N47D, M1V/H19Y/T36A/N42R/N53S, H19Y/L35P/T36A/N42D/T46I/V49A, Q3P/S9P/H19Y/N31S/T36A/R39H/N47R/K50E, M1V/H19Y/T36A/N42R/N53S, M1T/H19Y/T36A, M1V/S16N/H19Y/I22M/T36A, M1T/N11D/H19Y/T36A/N42S/V45A/N53S, N11D/S16G/H19Y/T36A/N47S/N53D, M1V/S9P/Q10P/S16G/H19Y/L26F/T36A/A43V/N53D, S16G/H19Y/T36A/V49A/N53D, S16G/T36A/A43T/S44G/V45M, M4V/S9P/S16G/T36A/Q38R, S9P/N11S/S16G/T36A/Q38R, N11D/E12K/S16R/T36A/T52M, M4V/T32I/T36A/Q38R/A43T/V45A/S48P, S9P/N11D/S16G/Q25R, M1T/A5T/S9P/S16G/Q25R/N31D/V49M, L2S/S9P/S16G/A20T/T32I/Q38R/N42D/T46A/S48L, S16G/Q25R/T46A, G6E/S9A/S16G/Q25R/N31D/N47S/T52M, H19Y/Q38R/T52M, N11D/H19Y/I22M/T32P/N47S/N53S, S16G/H19Y/T36A, S16G/H19Y/T36A/N53D, S9P/N11D/S16G/H19Y/T36A/N47S/N53D, Q3P/S9P/H19Y/N31S/T36A/R39H/N47R/K50E, M1V/L2S/M4T/N11D/H19Y/T36A, M1V/L2S/M4T/N11D/T36A, M1V/L2S/M4T/H19Y/T36I/V45A/V49M, M1V/L2S/M4T/N11D/H19Y/T36A, M1V/L2S/M4T/S9P/Q10R/H19Y/T36A/T46A/N47S, M1V/L2S/M4T/S16G/N31D/T32I/T36A, M1V/M4T/T36A/Q38R/N53K, M1T/N11D/H19Y/T36A/N42S/V45A/N53S, M1T/N31D/T32A/T36A/A38R/S44D/V49A/K50E, M1T/S9P/P23S/Q38R/N42S/S48P/V49A/A54V, H19Y/T36A/S44G, H19Y/T36A, and M4T/T36A/Q38R/N42S/S44G/T46A/N47K/S48P/T52A.

In some embodiments, the variant sBCMA as described herein comprises amino acid substitutions S16G/H19Y/T36A, and at least one further amino acid substitution selected from the group consisting of M1A, M1C, M1I, M1R, M1T, M1V, L2C, L2S, Q3P, Q3R, M4E, M4I, M4T, M4V, A5T, G6E, Q7R, S9A, S9F, S9P, Q10H, Q10P, Q10R, N11D, N11S, E12K, F14L, S16N, S16R, H19L, A20V, A20T, I22M, I22V, P23S, Q25R, L26F, S29A, N31D, N31S, T32A, T32I, T32P, L35S, L35P, T36I, T36P, Q38R, R39H, N42D, N42R, N42S, A43T, A43V, S44D, S44G, S44N, S44R, V45A, V45M, T46A, T46I, N47D, N47K, N47R, N47S, S48L, S48P, S48T, V49A, V49M, K50E, K50G, K50R, K50T, G51E, T52A, T52M, N53D, N53K, N53S, A54V, and A54T.

In some embodiments, the variant sBCMA as described herein comprises amino acid substitutions S16G/H19Y/T36A/N53D, and at least one further amino acid substitution selected from the group consisting of M1A, M1C, M1I, M1R, M1T, M1V, L2C, L2S, Q3P, Q3R, M4E, M4I, M4T, M4V, A5T, G6E, Q7R, S9A, S9F, S9P, Q10H, Q10P, Q10R, N11D, N11S, E12K, F14L, S16N, S16R, H19L, A20V, A20T, I22M, I22V, P23S, Q25R, L26F, S29A, N31D, N31S, T32A, T32I, T32P, L35S, L35P, T36I, T36P, Q38R, R39H, N42D, N42R, N42S, A43T, A43V, S44D, S44G, S44N, S44R, V45A, V45M, T46A, T46I, N47D, N47K, N47R, N47S, S48L, S48P, S48T, V49A, V49M, K50E, K50G, K50R, K50T, G51E, T52A, T52M, N53K, N53S, A54V, and A54T.

In some embodiments, the variant sBCMA as described herein comprises amino acid substitutions S9P/N11D/S16G/H19Y/T36A/N47S/N53D, and at least one further amino acid substitution selected from the group consisting of M1A, MIC, M1I, M1R, M1T, M1V, L2C, L2S, Q3P, Q3R, M4E, M4I, M4T, M4V, A5T, G6E, Q7R, S9A, S9F, Q10H, Q10P, Q10R, N11S, E12K, F14L, S16N, S16R, H19L, A20V, A20T, I22M, I22V, P23S, Q25R, L26F, S29A, N31D, N31S, T32A, T32I, T32P, L35S, L35P, T36I, T36P, Q38R, R39H, N42D, N42R, N42S, A43T, A43V, S44D, S44G, S44N, S44R, V45A, V45M, T46A, T46I, N47D, N47K, N47R, S48L, S48P, S48T, V49A, V49M, K50E, K50G, K50R, K50T, G51E, T52A, T52M, N53K, N53S, A54V, and A54T.

In some embodiments, the variant sBCMA as described herein comprises amino acid substitutions Q3P/S9P/H19Y/N31S/T36A/R39H/N47R/K50E, and at least one further amino acid substitution selected from the group consisting of M1A, M1C, M1I, M1R, M1T, M1V, L2C, L2S, Q3R, M4E, M4I, M4T, M4V, A5T, G6E, Q7R, S9A, S9F, Q10H, Q10P, Q10R, N11D, N11S, E12K, F14L, S16G, S16N, S16R, H19L, A20V, A20T, I22M, I22V, P23S, Q25R, L26F, S29A, N31D, T32A, T32I, T32P, L35S, L35P, T36I, T36P, Q38R, N42D, N42R, N42S, A43T, A43V, S44D, S44G, S44N, S44R, V45A, V45M, T46A, T46I, N47D, N47K, N47S, S48L, S48P, S48T, V49A, V49M, K50G, K50R, K50T, G51E, T52A, T52M, N53D, N53K, N53S, A54V, and A54T.

In some embodiments, the variant sBCMA as described herein comprises amino acid substitutions M1V/L2S/M4T/S16G/N31D/T32I/T36A, and at least one further amino acid substitution selected from the group consisting of M1A, MIC, M1I, M1R, M1T, L2C, Q3P, Q3R, M4E, M4I, M4V, A5T, G6E, Q7R, S9A, S9F, S9P, Q10H, Q10P, Q10R, N11D, N11S, E12K, F14L, S16N, S16R, H19L, H19Y, A20V, A20T, I22M, I22V, P23S, Q25R, L26F, S29A, N31S, T32A, T32P, L35S, L35P, T36I, T36P, Q38R, R39H, N42D, N42R, N42S, A43T, A43V, S44D, S44G, S44N, S44R, V45A, V45M, T46A, T46I, N47D, N47K, N47R, N47S, S48L, S48P, S48T, V49A, V49M, K50E, K50G, K50R, K50T, G51E, T52A, T52M, N53D, N53K, N53S, A54V, and A54T.

In some embodiments, the variant sBCMA as described herein has at least 90% sequence identity to SEQ ID NO: 67.

In some embodiments, the variant sBCMA as described herein has at least 90% sequence identity to SEQ ID NO: 68.

In some embodiments, the variant sBCMA as described herein has at least 90% sequence identity to SEQ ID NO: 69.

In some embodiments, the variant sBCMA as described herein has at least 90% sequence identity to SEQ ID NO: 49.

In some embodiments, the variant sBCMA as described herein has at least 90% sequence identity to SEQ ID NO: 74.

In some embodiments, the variant sBCMA as described herein has the amino acid sequence of SEQ ID NO: 67.

In some embodiments, the variant sBCMA as described herein has the amino acid sequence of SEQ ID NO: 68.

In some embodiments, the variant sBCMA as described herein has the amino acid sequence of SEQ ID NO: 69.

In some embodiments, the variant sBCMA as described herein has the amino acid sequence of SEQ ID NO: 49.

In some embodiments, the variant sBCMA as described herein has the amino acid sequence of SEQ ID NO: 74.

The clone Nos., amino acid substitutions as compared to the amino acid sequence of SEQ ID NO:1, and assigned SEQ ID NOs of exemplary variant sBCMA proteins are shown in Table 2.

TABLE 2
The clone numbers, amino acid substitutions as compared to
the amino acid sequence of SEQ ID NO: 1, and the assigned
SEQ ID NOs. of exemplary variant sBCMA proteins/domains.

| | | SEQ ID |
|---|---|---|
| Clone Nos. | Amino Acid Substitutions as compared to SEQ ID NO: 1 | Nos. |
| # 1 | L2S/S9P/E12K/N31D/T36A/N42S/N53S | SEQ ID |
| | | NO: 2 |
| # 2 | M1V/T32P/T36A/T46I/N53D/A54V | SEQ ID |
| | | NO: 3 |
| # 3 | Q3R/S16N/T36A/A43T | SEQ ID |
| | | NO: 4 |
| # 4 | F14L/S16G/T36A/V45A/N47D | SEQ ID |
| | | NO: 5 |
| # 5 | M1T/M4V/S9F/S16G/T32A/Q38R | SEQ ID |
| | | NO: 6 |
| # 6 | M1A/S9A/Q38R | SEQ ID |
| | | NO: 7 |
| # 7 | G6E/Q25R/Q38R | SEQ ID |
| | | NO: 8 |
| # 8 | M1V/M4I/G6E/S9P/N11D/V49M/T52M/A54V | SEQ ID |
| | | NO: 9 |
| # 9 | N11D/S16G/N31S | SEQ ID |
| | | NO: 10 |
| # 10 and 70 | N11D/H19Y/I22M/T32P/N47S/N53S | SEQ ID |
| | | NO: 11 |
| # 11 | G6E/Q7R/H19Y/L35S | SEQ ID |
| | | NO: 12 |
| # 12 | H19Y/N42D/S48P/T52A | SEQ ID |
| | | NO: 13 |
| # 13 | M1V/N31D/T32I/T36A | SEQ ID |
| | | NO: 14 |
| # 14 | M1V/A5T/H19L/T36A | SEQ ID |
| | | NO: 15 |
| # 15 | M1T/N31D/T32A/T36A/Q38R/S44D/V49A/K50E | SEQ ID |
| | | NO: 16 |
| # 16 | M1V/T36A/Q38R/A43V | SEQ ID |
| | | NO: 17 |
| # 17 | M1V/L2S/S9P/Q10H/T36A/Q38R/K50G | SEQ ID |
| | | NO: 18 |
| # 18 | T36A/Q38R/N53S | SEQ ID |
| | | NO: 19 |
| # 19 | M1T/L2S/L35P/T36A/Q38R/T46A/K50R | SEQ ID |
| | | NO: 20 |
| # 20 | A5T/A20V/T36A/Q38R | SEQ ID |
| | | NO: 21 |
| # 21 | M1T/S16G/I22V/T36A/S44G/T46A/V49A | SEQ ID |
| | | NO: 22 |
| # 22 & 73 | S16G/T36A | SEQ ID |
| | | NO: 23 |
| # 23 and 25 | M1I/N11D/S16G/I22M/S29A/T36A/S44G/K50R | SEQ ID |
| | | NO: 24 |
| # 24 | M1C/L2C/Q3R/M4E/N11D/S16G/T36P | SEQ ID |
| | | NO: 25 |
| # 26 | N11D/N31D/T32I/T36A/S44N/N47D/N53D | SEQ ID |
| | | NO: 26 |
| # 27 | M1R/L2C/Q3R/N11D/H19Y/T36A/N42S/V45A/N53S | SEQ ID |
| | | NO: 27 |
| # 28 and | H19Y/T36A/S44G | SEQ ID |
| 116 | | NO: 28 |
| # 29 | H19Y/T32I/T36A/V49A | SEQ ID |
| | | NO: 29 |
| # 30 | H19Y/N31S/T36A/V45A | SEQ ID |
| | | NO: 30 |
| # 31 | H19Y/N31S/T36A | SEQ ID |
| | | NO: 31 |
| # 32 | H19Y/T36P/T52A | SEQ ID |
| | | NO: 32 |
| # 33 | H19Y/N31D/T52M | SEQ ID |
| | | NO: 33 |
| # 34 | M1V/H19Y/V45M | SEQ ID |
| | | NO: 34 |
| # 35 | S16G/H19Y/N47D | SEQ ID |
| | | NO: 35 |
| # 36 | S16G/H19Y/K50T | SEQ ID |
| | | NO: 36 |
| # 37 | S16G/H19Y/S44N/K50R | SEQ ID |
| | | NO: 37 |
| # 38 | N11D/H19Y/S48T | SEQ ID |
| | | NO: 38 |
| # 39 | S9P/N11D/S16R/T32A/Q38R/S44G/T46I/T52A/N53D/A54T | SEQ ID |
| | | NO: 39 |
| # 40 | N11D/S16G/S44R | SEQ ID |
| | | NO: 40 |
| # 41 | H19L/T32A/S44G/G51E/T52A | SEQ ID |
| | | NO: 41 |
| # 42 | S16N/H19Y/T36A/K50R | SEQ ID |
| | | NO: 42 |
| # 43 | M1V/H19Y/T36A/R39H/T46A | SEQ ID |
| | | NO: 43 |
| # 44 | M1V/H19Y/T36A | SEQ ID |
| | | NO: 44 |
| # 45 | H19Y/T36A/N42D/N47S/S48P | SEQ ID |
| | | NO: 45 |
| # 46 | M1V/H19Y/T36A/S44G/N47D | SEQ ID |
| | | NO: 46 |
| # 47 and 50 | M1V/H19Y/T36A/N42R/N53S | SEQ ID |
| | | NO: 47 |
| # 48 | H19Y/L35P/T36A/N42D/T46I/V49A | SEQ ID |
| | | NO: 48 |
| # 49, 75-94 | Q3P/S9P/H19Y/N31S/T36A/R39H/N47R/K50E | SEQ ID |
| | | NO: 49 |
| # 51 | M1T/H19Y/T36A | SEQ ID |
| | | NO: 50 |
| # 52 | M1V/S16N/H19Y/I22M/T36A | SEQ ID |
| | | NO: 51 |
| # 53, 54 and | M1T/N11D/H19Y/T36A/N42S/V45A/N53S | SEQ ID |
| 113 | | NO: 52 |
| # 55 and 56 | N11D/S16G/H19Y/T36A/N47S/N53D | SEQ ID |
| | | NO: 53 |
| # 57 | M1V/S9P/Q10P/S16G/H19Y/L26F/T36A/A43V/N53D | SEQ ID |
| | | NO: 54 |
| # 58 | S16G/H19Y/T36A/V49A/N53D | SEQ ID |
| | | NO: 55 |
| # 59 | S16G/T36A/A43T/S44G/V45M | SEQ ID |
| | | NO: 56 |
| # 60 | M4V/S9P/S16G/T36A/Q38R | SEQ ID |
| | | NO: 57 |
| # 61 | S9P/N11S/S16G/T36A/Q38R | SEQ ID |
| | | NO: 58 |
| # 62 | N11D/E12K/S16R/T36A/T52M | SEQ ID |
| | | NO: 59 |
| # 63 | M4V/T32I/T36A/Q38R/A43T/V45A/S48P | SEQ ID |
| | | NO: 60 |
| # 64 | S9P/N11D/S16G/Q25R | SEQ ID |
| | | NO: 61 |
| # 65 | M1T/A5T/S9P/S16G/Q25R/N31D/V49M | SEQ ID |
| | | NO: 62 |
| # 66 | L2S/S9P/S16G/A20T/T32I/Q38R/N42D/T46A/S48L | SEQ ID |
| | | NO: 63 |
| # 67 | S16G/Q25R/T46A | SEQ ID |
| | | NO: 64 |
| # 68 | G6E/S9A/S16G/Q25R/N31D/N47S/T52M | SEQ ID |
| | | NO: 65 |
| # 69 | H19Y/Q38R/T52M | SEQ ID |
| | | NO: 66 |
| # 71 | S16G/H19Y/T36A/N53D | SEQ ID |
| | | NO: 67 |
| # 72 | S16G/H19Y/T36A | SEQ ID |
| | | NO: 68 |
| # 74 | S9P/N11D/S16G/H19Y/T36A/N47S/N53D | SEQ ID |
| | | NO: 69 |
| # 95, and | M1V/L2S/M4T/N11D/H19Y/T36A | SEQ ID |
| 101-103 | | NO: 70 |
| # 96-99 | M1V/L2S/M4T/N11D/T36A | SEQ ID |
| | | NO: 71 |
| # 100 | M1V/L2S/M4T/H19Y/T36I/V45A/V49M | SEQ ID |
| | | NO: 72 |
| # 104 and | M1V/L2S/M4T/S9P/Q10R/H19Y/T36A/T46A/N47S | SEQ ID |
| 105 | | NO: 73 |
| # 106-111 | M1V/L2S/M4T/S16G/N31D/T32I/T36A | SEQ ID |
| | | NO: 74 |
| # 112 | M1V/M4T/T36A/Q38R/N53K | SEQ ID |
| | | NO: 75 |
| # 114 | M1T/N31D/T32A/T36A/A38R/S44D/V49A/K50E | SEQ ID |
| | | NO: 76 |
| # 115 | M1T/S9P/P23S/Q38R/N42S/S48P/V49A/A54V | SEQ ID |
| | | NO: 77 |
| # 117 | H19Y/T36A | SEQ ID |
| | | NO: 78 |
| # 118 | M4T/T36A/Q38R/N42S/S44G/T46A/N47K/S48P/T52A | SEQ ID |
| | | NO: 79 |

In some embodiments, the variant sBCMA as described herein exhibits enhanced binding affinity for APRIL or BAFF as compared to SEQ ID NO: 1.

In some embodiments, the variant sBCMA as described herein exhibits enhanced binding affinity for APRIL and BAFF as compared to SEQ ID NO:1.

In some embodiments, the sBCMA variant—Fc fusion protein as described herein has the amino acid sequence of SEQ ID NO:80.

In some embodiments, the sBCMA variant—Fc fusion protein as described herein has the amino acid sequence of SEQ ID NO:81.

In some embodiments, the sBCMA variant—Fc fusion protein as described herein has the amino acid sequence of SEQ ID NO:82.

In some embodiments, the sBCMA variant—Fc fusion protein as described herein has the amino acid sequence of SEQ ID NO:83.

In some embodiments, the sBCMA variant—Fc fusion protein as described herein has the amino acid sequence of SEQ ID NO:84.

### 2. Assays to Measure Binding Affinity

As outlined herein, the present invention provides sBCMA variants and fusion proteins comprising these variants that exhibit increased binding affinity for either or both of human APRIL and/or human BAFF. In this context, increased binding affinity is compared to the human wild type BCMA or SEQ ID NO:1 in vitro or ex vivo studies as outlined below. In some embodiments, the variant sBCMA domain as described herein has a binding affinity for TGF family member (e.g., APRIL and/or BAFF) that is stronger than the wild-type sBCMA polypeptide/domain and/or SEQ ID NO:1. In some embodiments, the variant sBCMA domain has a binding affinity for APRIL and/or BAFF that is at least 1.4-fold, 1.5-fold, 1.6-fold, 1.7-fold, 1.8-fold, 1.9-fold, 2-fold, 3-fold, 4-fold, 5-fold, 10-fold, 50-fold, 100-fold, 200-fold or greater than that of the wild-type sBCMA and/or SEQ ID NO:1.

The ability of an sBCMA variant to bind to APRIL and/or BAFF can be determined, for example, by the ability of the putative ligand to bind to APRIL and/or BAFF coated on an assay plate. Alternatively, binding affinity of an sBCMA (variant) for APRIL and/or BAFF can be determined by displaying the sBCMA (variant) on a microbial cell surface, e.g., a yeast cell surface and detecting the bound complex by, for example, flow cytometry (see, Example 3). The binding affinity of sBCMA (variant) for APRIL and/or BAFF can be measured using any appropriate method as would be understood by those skilled in the art including, but not limited to, radioactive ligand binding assays, non-radioactive (fluorescent) ligand binding assays, surface plasmon resonance (SPR), such as Biacore™, Octet™, plasmon-waveguide resonance (PWR), thermodynamic binding assays, whole cell ligand-binding assays, and structure-based ligand binding assays.

### 3. Formats of the Fusion Proteins

As described herein, the format of the fusion protein can take on several configurations, with the component domains switching order in the protein (from N- to C-terminal). In one embodiment, a fusion protein comprises, from N- to C-terminus, a variant sBCMA domain-domain linker-Fc domain. In some embodiments, a fusion protein comprises, from N- to C-terminus, Fc domain-domain linker—variant sBCMA domain. In some embodiments, a linker is not used, in which case the fusion protein comprises from N- to C-terminus, either variant sBCMA domain-Fc domain or Fc domain—variant sBCMA domain. Note that in some cases, the same fusion protein can be labeled somewhat differently. For example, in the case in which the Fc domain includes a hinge domain, a fusion protein comprising variant sBCMA domain-Fc domain still includes a linker in the form of the hinge domain. Alternatively, this same protein may not have the hinge domain included in the Fc domain, in which case the fusion protein comprises variant sBCMA domain-CH2-CH3.

Thus, in some embodiments, the present disclosure provides a variant sBCMA—Fc fusion protein as described herein, where the Fc domain comprises a hinge domain and the variant sBCMA domain is linked with the Fc domain by the hinge domain: variant sBCMA domain-hinge domain-CH2-CH3.

In some embodiments, the present disclosure provides a variant sBCMA—Fc fusion proteins as described above, where the Fc domain comprises a hinge domain and the variant sBCMA domain is linked with the Fc domain by an additional linker as described herein. That is, the fusion protein can be, from N- to C-terminal: variant sBCMA domain-domain linker-hinge domain-CH2-CH3; variant sBCMA domain-domain linker-CH2-CH3; hinge domain-CH2-CH3-domain linker-variant sBCMA domain or CH2-CH3-domain linker-variant sBCMA domain.

In some embodiments, the present disclosure provides variant sBCMA—Fc fusion proteins as described above, where the Fc domain does not comprise a hinge domain and the variant sBCMA domain is linked with the Fc domain by a domain linker (e.g. non-hinge) as described herein.

In some embodiments, the present disclosure provides a composition comprising an sBCMA variant—Fc fusion protein comprising:
a) a variant sBCMA domain comprising at least one amino acid substitution as compared to SEQ ID NO:1, wherein said amino acid substitution is at a position number selected from the group consisting of 1, 2, 3, 4, 5, 6, 7, 9, 10, 11, 12, 14, 16, 19, 20, 22, 23, 25, 26, 29, 31, 32, 35, 36, 38, 39, 42, 43, 44, 45, 46, 47, 48, 49, 50, 51, 52, 53, and 54, wherein the numbering is according to the EU index;
b) an optional linker; and
c) an Fc domain.

In some embodiments, the sBCMA variant—Fc fusion protein as described herein comprises, from N- to C-terminal:
a) said variant sBCMA domain;
b) said optional linker; and
c) said Fc domain.

In some embodiments, the sBCMA variant—Fc fusion protein as described herein comprises, from N- to C-terminal:
a) said Fc domain;
b) said optional linker; and
c) said variant sBCMA domain.

In some embodiments, a variant sBCMA domain of the sBCMA variant—Fc fusion protein as described herein serves to increase the binding affinity for APRIL and/or BAFF. In various embodiments, a (variant) Fc domain of the sBCMA variant—Fc fusion protein as described herein increases the half-life of the fusion protein. In a number of embodiments, fusion proteins are used to treat tumors/cancers, fibrosis and/or immunomodulatory diseases.

The names of the designated proteins/protein domains and corresponding amino acid sequences are listed in Table 3, respectively.

TABLE 3
SEQ ID numbers, descriptions and
corresponding amino acid sequences.

| SEQ ID NO | |
|---|---|
| (Description) | Amino Acid Sequence |
| SEQ ID NO: 1 | MLQMAGQCSQNEYFDSLLHACIPCQLRCSSNTP |
| (sBCMA WT ECD) | PLTCQRYCNASVTNSVKGTNA |
| SEQ ID NO: 87 | IEGRMD |
| (domain linker) | |
| SEQ ID NO: 88 | GGGGS |
| (domain linker) | |

In some embodiments, the variant sBCMA domain as described herein includes amino acid substitution(s), deletion(s) or insertion(s) or any combination thereof to the amino acid sequence of SEQ ID NO:1 that increases its binding activity to either APRIL, BAFF or both as compared to wild-type sBCMA.

The present disclosure provides variant sBCMA domains comprising at least one amino acid substitution at one or more (e.g., 2, 3, 4, 5, 6, 7, 8, 9 or 10) positions as compared to the amino acid sequence of SEQ ID NO:1. In some embodiments, the variant sBCMA domain has at least 80%, at least 85%, at least 90%, at least 95%, at least 96%, at least 97%, at least 98%, or at least 99%, but less than 100% sequence identity to the parent sBCMA domain. In some embodiments, a parent sBCMA domain has the amino acid sequence of SEQ ID NO:1. In some embodiments, a variant sBCMA domain has at least 90%, at least 91%, at least 92%, at least 93%, at least 94%, at least 95%, at least 96%, at least 97%, at least 98%, or at least 99%, but less than 100% sequence identity to SEQ ID NO:1. In some embodiments, as noted herein, a variant sBCMA domain can have N-terminal and/or C terminal truncations compared to wild type sBCMA as long as the truncated variant sBCMA retains biological activity (e.g. binding to APRIL and/or BAFF), as measured by one of the binding assays outlined herein. To be clear, the variant BCMA domain of the present invention has at least one amino acid substitution and thus is not the amino acid sequence of SEQ ID NO:1.

In some embodiments, the variant sBCMA domain as described herein has amino acid substitution(s) at one position, two positions, three positions, four positions, five positions, six positions, seven positions, eight positions, nine positions, or ten positions.

In certain embodiments, the binding affinity of the variant sBCMA domain as described herein for APRIL and/or BAFF is increased by at least about 0.4%, 0.5%, 1%, 2%, 3%, 4%, 5%, 6%, 7%, 8%, 9%, 10%1, 15%, 20%, 25%, 30%, 35%, 40%, 45%, 50% or higher as compared to that of the wild-type sBCMA. In other embodiments, variant BCMA domains of the present invention have a binding affinity of less than about 1×10⁻⁸ M, 1×10⁻⁹ M, 1×10⁻¹⁰ M, 1×10⁻¹² M or 1×10⁻¹⁵ M for APRIL and/or BAFF. In yet other embodiments, variant BCMA domains as described herein inhibit or compete with wild-type sBCMA binding to APRIL and/or BAFF either in vivo, in vitro or both.

In some embodiments, the variant sBCMA domain as described herein comprises at least one amino acid substitution as compared to SEQ ID NO:1, wherein said amino acid substitution is at a position number selected from the group consisting of 1, 2, 3, 4, 5, 6, 7, 9, 10, 11, 12, 14, 16, 19, 20, 22, 23, 25, 26, 29, 31, 32, 35, 36, 38, 39, 42, 43, 44, 45, 46, 47, 48, 49, 50, 51, 52, 53, and 54, wherein the numbering is according to the EU index.

In some embodiments, the variant sBCMA domain as described herein has at least 80%, at least 85%, at least 90%, or at least 95% sequence identity to SEQ ID NO: 1.

In some embodiments, the variant sBCMA domain as described herein comprises amino acid substitution(s) selected from the group consisting of M1A, M1C, M1I, M1R, M1T, M1V, L2C, L2S, Q3P, Q3R, M4E, M4I, M4T, M4V, A5T, G6E, Q7R, S9A, S9F, S9P, Q10H, Q10P, Q10R, N11D, N11S, E12K, F14L, S16G, S16N, S16R, H19L, H19Y, A20V, A20T, I22M, I22V, P23S, Q25R, L26F, S29A, N31D, N31S, T32A, T32I, T32P, L35S, L35P, T36A, T36I, T36P, Q38R, R39H, N42D, N42R, N42S, A43T, A43V, S44D, S44G, S44N, S44R, V45A, V45M, T46A, T46I, N47D, N47K, N47R, N47S, S48L, S48P, S48T, V49A, V49M, K50E, K50G, K50R, K50T, G51E, T52A, T52M, N53D, N53K, N53S, A54V, and A54T.

In some embodiments, the variant sBCMA domain as described herein comprises amino acid substitution(s) selected from the group consisting of M1V, L2S, Q3P, M4T, S9P, N11D, S16G, H19Y, N31S, N31D, T32I, T36A, R39H, N47S, K50E, and N53E.

In some embodiments, the variant sBCMA domain as described herein comprises amino acid substitution(s) selected from the group consisting of S16G, H19Y and T36A.

In some embodiments, the variant sBCMA domain as described herein comprises amino acid substitutions selected from the group consisting of L2S/S9P/E12K/N31D/T36A/N42S/N53S, M1V/T32P/T36A/T46I/N53D/A54V, Q3R/S16N/T36A/A43T, F14L/S16G/T36A/V45A/N47D, M1T/M4V/S9F/S16G/T32A/Q38R, M1A/S9A/Q38R, G6E/Q25R/Q38R, M1V/M4I/G6E/S9P/N11D/V49M/T52M/A54V, N11D/S16G/N31S, N11D/H19Y/I22M/T32P/N47S/N53S, G6E/Q7R/H19Y/L35S, H19Y/N42D/S48P/T52A, M1V/N31D/T32I/T36A, M1V/A5T/H19L/T36A, M1T/N31D/T32A/T36A/Q38R/S44D/V49A/K50E, M1V/T36A/Q38R/A43V, M1V/L2S/S9P/Q10H/T36A/Q38R/K50G, T36A/Q38R/N53S, M1T/L2S/L35P/T36A/Q38R/T46A/K50R, A5T/A20V/T36A/Q38R, M1T/S16G/I22V/T36A/S44G/T46A/V49A, S16G/T36A, M1I/N11D/S16G/I22M/S29A/T36A/S44G/K50R, M1C/L2C/Q3R/M4E/N11D/S16G/T36P, M1I/N11D/S16G/I22M/S29A/T36A/S44G/K50R, N11D/N31D/T32I/T36A/S44N/N47D/N53D, M1R/L2C/Q3R, H19Y/T36A/S44G, H19Y/T32I/T36A/V49A, H19Y/N31S/T36A/V45A, H19Y/N31S/T36A, H19Y/T36P/T52A, H19Y/N31D/T52M, M1V/H19Y/V45M, S16G/H19Y/N47D, S16G/H19Y/K50T, S16G/H19Y/S44N/K50R, N11D/H19Y/S48T, S9P/N11D/S16R/T32A/Q38R/S44G/T46I/T52A/N53D/A54T, N11D/S16G/S44R, H19L/T32A/S44G/G51E/T52A, S16N/H19Y/T36A/K50R, M1V/H19Y/T36A/R39H/T46A, M1V/H19Y/T36A, H19Y/T36A/N42D/N47S/S48P, M1V/H19Y/T36A/S44G/N47D, M1V/H19Y/T36A/N42R/N53S, H19Y/L35P/T36A/N42D/T46I/V49A, Q3P/S9P/H19Y/N31S/T36A/R39H/N47R/K50E, M1V/H19Y/T36A/N42R/N53S, M1T/H19Y/T36A, M1V/S16N/H19Y/I22M/T36A, M1T/N11D/H19Y/T36A/N42S/V45A/N53S, N11D/S16G/H19Y/T36A/N47S/N53D, M1V/S9P/Q10P/S16G/H19Y/L26F/T36A/A43V/N53D, S16G/H19Y/T36A/V49A/N53D, S16G/T36A/A43T/S44G/V45M, M4V/S9P/S16G/T36A/Q38R, S9P/N11S/S16G/T36A/Q38R, N11D/E12K/S16R/T36A/T52M, M4V/T32I/T36A/Q38R/A43T/V45A/S48P, S9P/N11D/S16G/Q25R, M1T/A5T/S9P/S16G/Q25R/N31D/V49M, L2S/S9P/S16G/A20T/T32I/Q38R/N42D/T46A/S48L, S16G/Q25R/T46A, G6E/S9A/S16G/Q25R/N31D/N47S/T52M, H19Y/Q38R/T52M, N11D/H19Y/I22M/T32P/N47S/N53S, S16G/H19Y/T36A, S16G/H19Y/T36A/N53D, S9P/N11D/S16G/H19Y/T36A/N47S/N53D, Q3P/S9P/H19Y/N31S/T36A/R39H/N47R/K50E, M1V/L2S/M4T/N11D/H19Y/T36A, M1V/L2S/M4T/N11D/T36A, M1V/L2S/M4T/H19Y/T36I/V45A/V49M, M1V/L2S/M4T/N11D/H19Y/T36A, M1V/L2S/M4T/S9P/Q10R/H19Y/T36A/T46A/N47S, M1V/L2S/M4T/S16G/N31D/T32I/T36A, M1V/M4T/T36A/Q38R/N53K, M1T/N11D/H19Y/T36A/N42S/V45A/N53S, M1T/N31D/T32A/T36A/A38R/S44D/V49A/K50E, M1T/S9P/P23S/Q38R/N42S/S48P/V49A/A54V, H19Y/T36A/S44G, H19Y/T36A, and M4T/T36A/Q38R/N42S/S44G/T46A/N47K/S48P/T52A.

In some embodiments, the variant sBCMA domain as described herein comprises amino acid substitutions S16G/H19Y/T36A, and at least one further amino acid substitution selected from the group consisting of M1A, MIC, M1I, M1R, M1T, M1V, L2C, L2S, Q3P, Q3R, M4E, M4I, M4T, M4V, A5T, G6E, Q7R, S9A, S9F, S9P, Q10H, Q10P, Q10R, N11D, N11S, E12K, F14L, S16N, S16R, H19L, A20V, A20T, I22M, I22V, P23S, Q25R, L26F, S29A, N31D, N31S, T32A, T32I, T32P, L35S, L35P, T36I, T36P, Q38R, R39H, N42D, N42R, N42S, A43T, A43V, S44D, S44G, S44N, S44R, V45A, V45M, T46A, T46I, N47D, N47K, N47R, N47S, S48L, S48P, S48T, V49A, V49M, K50E, K50G, K50R, K50T, G51E, T52A, T52M, N53D, N53K, N53S, A54V, and A54T.

In some embodiments, the variant sBCMA domain as described herein comprises amino acid substitutions S16G/H19Y/T36A/N53D, and at least one further amino acid substitution selected from the group consisting of M1A, M1C, M1I, M1R, M1T, M1V, L2C, L2S, Q3P, Q3R, M4E, M4I, M4T, M4V, A5T, G6E, Q7R, S9A, S9F, S9P, Q10H, Q10P, Q10R, N11D, N11S, E12K, F14L, S16N, S16R, H19L, A20V, A20T, I22M, I22V, P23S, Q25R, L26F, S29A, N31D, N31S, T32A, T32I, T32P, L35S, L35P, T36I, T36P, Q38R, R39H, N42D, N42R, N42S, A43T, A43V, S44D, S44G, S44N, S44R, V45A, V45M, T46A, T46I, N47D, N47K, N47R, N47S, S48L, S48P, S48T, V49A, V49M, K50E, K50G, K50R, K50T, G51E, T52A, T52M, N53K, N53S, A54V, and A54T.

In some embodiments, the variant sBCMA domain as described herein comprises amino acid substitutions S9P/N11D/S16G/H19Y/T36A/N47S/N53D, and at least one further amino acid substitution selected from the group consisting of M1A, M1C, M1I, M1R, M1T, M1V, L2C, L2S, Q3P, Q3R, M4E, M4I, M4T, M4V, A5T, G6E, Q7R, S9A, S9F, Q10H, Q10P, Q10R, N11S, E12K, F14L, S16N, S16R, H19L, A20V, A20T, I22M, I22V, P23S, Q25R, L26F, S29A, N31D, N31S, T32A, T32I, T32P, L35S, L35P, T36I, T36P, Q38R, R39H, N42D, N42R, N42S, A43T, A43V, S44D, S44G, S44N, S44R, V45A, V45M, T46A, T46I, N47D, N47K, N47R, S48L, S48P, S48T, V49A, V49M, K50E, K50G, K50R, K50T, G51E, T52A, T52M, N53K, N53S, A54V, and A54T.

In some embodiments, the variant sBCMA domain as described herein comprises amino acid substitutions Q3P/S9P/H19Y/N31S/T36A/R39H/N47R/K50E, and at least one further amino acid substitution selected from the group consisting of M1A, M1C, M1I, M1R, M1T, M1V, L2C, L2S, Q3R, M4E, M4I, M4T, M4V, A5T, G6E, Q7R, S9A, S9F, Q10H, Q10P, Q10R, N11D, N11S, E12K, F14L, S16G, S16N, S16R, H19L, A20V, A20T, I22M, I22V, P23S, Q25R, L26F, S29A, N31D, T32A, T32I, T32P, L35S, L35P, T36I, T36P, Q38R, N42D, N42R, N42S, A43T, A43V, S44D, S44G, S44N, S44R, V45A, V45M, T46A, T46I, N47D, N47K, N47S, S48L, S48P, S48T, V49A, V49M, K50G, K50R, K50T, G51E, T52A, T52M, N53D, N53K, N53S, A54V, and A54T.

In some embodiments, the variant sBCMA domain as described herein comprises amino acid substitutions M1V/L2S/M4T/S16G/N31D/T32I/T36A, and at least one further amino acid substitution selected from the group consisting of M1A, M1C, M1I, M1R, M1T, L2C, Q3P, Q3R, M4E, M4I, M4V, A5T, G6E, Q7R, S9A, S9F, S9P, Q10H, Q10P, Q10R, N11D, N11S, E12K, F14L, S16N, S16R, H19L, H19Y, A20V, A20T, I22M, I22V, P23S, Q25R, L26F, S29A, N31S, T32A, T32P, L35S, L35P, T36I, T36P, Q38R, R39H, N42D, N42R, N42S, A43T, A43V, S44D, S44G, S44N, S44R, V45A, V45M, T46A, T46I, N47D, N47K, N47R, N47S, S48L, S48P, S48T, V49A, V49M, K50E, K50G, K50R, K50T, G51E, T52A, T52M, N53D, N53K, N53S, A54V, and A54T.

In some embodiments, the variant sBCMA domain as described herein has at least 90% sequence identity to SEQ ID NO: 67.

In some embodiments, the variant sBCMA domain as described herein has at least 90% sequence identity to SEQ ID NO: 68.

In some embodiments, the variant sBCMA domain as described herein has at least 90% sequence identity to SEQ ID NO: 69.

In some embodiments, the variant sBCMA domain as described herein has at least 90% sequence identity to SEQ ID NO: 49.

In some embodiments, the variant sBCMA domain as described herein has at least 90% sequence identity to SEQ ID NO: 74.

In some embodiments, the variant sBCMA domain as described herein has SEQ ID NO: 67.

In some embodiments, the variant sBCMA domain as described herein has SEQ ID NO: 68.

In some embodiments, the variant sBCMA domain as described herein has SEQ ID NO: 69.

In some embodiments, the variant sBCMA domain as described herein has SEQ ID NO: 49.

In some embodiments, the variant sBCMA domain as described herein has SEQ ID NO: 74.

The Clone Nos., amino acid substitutions as compared to the amino acid sequence of SEQ ID NO:1 and assigned SEQ ID NOs of exemplary variant sBCMA domains are shown in Table 2.

In some embodiments, the variant sBCMA domain as described herein exhibits enhanced binding affinity for APRIL as compared to SEQ ID NO:1.

In some embodiments, the variant sBCMA domain as described herein exhibits enhanced binding affinity for BAFF as compared to SEQ ID NO:1.

In some embodiments, the variant sBCMA domain as described herein exhibits enhanced binding affinity for APRIL and BAFF as compared to SEQ ID NO: 1.

### 4. Fc Domains

As discussed herein, in addition to sBCMA variant domains described above, the fusion proteins of the invention also include Fc domains of antibodies that generally are based on the IgG class, which has several subclasses, including, but not limited to IgG1, IgG2, and IgG3. As described herein, an Fc domain optionally includes the hinge domain of an IgG antibody.

Human IgG Fe domains are of particular use in the present invention, and can be derived from the Fc domain from human IgG1, IgG2, or IgG3. In general, IgG1 and IgG2 are used more frequently than IgG3.

An Fc domain of a human IgG protein included in the fusion protein of the present invention can confer a significant increase in half-life of the fusion protein, and can provide additional binding or interaction with the Ig molecules. In some embodiments, an sBCMA variant—Fc fusion protein can facilitate purification, multimerization, binding and neutralizing other molecules as compared to a monomeric variant sBCMA polypeptide.

Fe domains can also contain Fc variants to alter function as needed. However, in accordance with many embodiments, Fc variants generally need to retain both the ability to form dimers as well as the ability to bind FcRn. Thus, while many of the embodiments herein rely on the use of a human IgG1 domain, Fc variants can be made to augment or abrogate function in other IgG domains. Thus, for example, ablation variants that reduce or eliminate effector function in IgG1 or IgG2 can be used, and/or FcRn variants that confer tighter binding to the FcRn receptor can be used, as will be appreciated by those in the art.

In one embodiment, an Fc domain is a human IgG Fc domain or a variant human IgG Fc domain.

In another embodiment, an Fc domain is human IgG1 Fc domain.

In a further embodiment, an Fc domain comprises the hinge-CH2-CH3 of human IgG1.

In another embodiment, an Fc domain comprises the CH2-CH3 of human IgG1.

In some embodiments, Fe domains can be the Fe domains from other IgGs than IgG1, such as human IgG2 or IgG3. In general, IgG2 is used more frequently than IgG3.

In an additional embodiment, an Fc domain is a variant human IgG Fc domain. However, the variant Fe domains herein still retain the ability to form a dimer with another Fc domain as measured using known, as well as the ability to bind to FcRn, as this contributes significantly to the increase in serum half life of the fusion proteins herein.

The variant IgG Fc domain can include an addition, deletion, substitution or any combination thereof compared with the parent human IgG Fc domain.

In some embodiments, variant human IgG Fe domains of the present invention can have at least about 80%, 85%, 90%, 95%, 95%, 97%, 98% or 99% identity to the corresponding parental human IgG Fc domain (using the identity algorithms discussed above, with one embodiment utilizing the BLAST algorithm as is known in the art, using default parameters).

In some embodiments, variant human IgG Fc domains of the present invention can have from 1, 2, 3, 4, 5, 6, 7, 8, 9, 10, 11, 12, 13, 14, 15, 16, 11, 12, 13, 14, 15, 16, 17, 18, 19 or 20 amino acid sequence modifications as compared to the parental human IgG Fc domains.

In some embodiments, the Fc domain as described herein is a human IgG Fc domain or a variant human IgG Fc domain.

In some embodiments, the Fc domain as described herein comprises the hinge-CH2-CH3 of human IgG1.

In some embodiments, the Fc domain as described herein is a variant human IgG Fc domain. 5. Linkers

The fusion proteins of the invention can include optional linkers to connect the sBCMA domain to the Fc domain.

By “linker” or “linker peptide” as used herein have a length that is adequate to link two molecules in such a way that they assume the correct conformation relative to one another so that they retain the desired activity. In one embodiment, the linker is from about 1 to 20 amino acids in length, preferably about 1 to 10 amino acids in length. In one embodiment, linkers of 4 to 10 amino acids in length may be used. Useful linkers include IEGRMD or glycine-serine polymers, including for example (GS)n, (GSGGS)n, (GGGGS)n, and (GGGS)n, where n is an integer of at least one (and generally from 3 to 4), glycine-alanine polymers, alanine-serine polymers, and other flexible linkers. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers.

In some embodiments, the linker is a “domain linker”, used to link any two domains as outlined herein together, such as to link the variant sBCMA domain with Fc domain. As discussed above, many suitable linkers can be used to allow for recombinant attachment of the two domains with sufficient length and flexibility to allow each domain to retain its biological function. As discussed herein, a particularly useful domain linker is an IEGRMD linker joined to the hinge domain of IgG1.

In various embodiments, two domains (e.g. the sBCMA variant domain and the Fc domain) are generally linked using a domain linker as described herein. In many embodiments, two domains are attached using a flexible linker in such a way that the two domains can act independently. Flexible linkage can be accomplished in a variety of ways, using traditional linkers and/or the hinge linker.

In some embodiments, the linker as described herein is IEGRMD (SEQ ID NO:87).

In some embodiments, the linker as described herein is GGGGS (SEQ ID NO:88).

In some embodiments, a hinge domain of a human IgG antibody is used. In some cases, a hinge domain can contain amino acid substitutions as well.

In some embodiments, a domain linker is a combination of a hinge domain and a flexible linker, such as an IgG1 hinge with an IEGRMD linker.

In one embodiment, a linker is from about 1 to 50 amino acids in length, preferably about 1 to 30 amino acids in length and more preferably about 4 to 10 amino acids. 6. Particular Embodiments of the Invention

In some embodiments, an sBCMA variant—Fc fusion protein exhibits at least 90%, 95%, 96%, 97%, 98% or 99% identity to SEQ ID NO:80.

In some embodiments, an sBCMA variant—Fc fusion protein exhibits at least 90%, 95%, 96%, 97%, 98% or 99% identity to SEQ ID NO:81.

In some embodiments, an sBCMA variant—Fc fusion protein exhibits at least 90%, 95%, 96%, 97%, 98% or 99% identity to SEQ ID NO:82.

In some embodiments, an sBCMA variant—Fc fusion protein exhibits at least 90%, 95%, 96%, 97%, 98% or 99% identity to SEQ ID NO:83.

In some embodiments, an sBCMA variant—Fc fusion protein exhibits at least 90%, 95%, 96%, 97%, 98% or 99% identity to SEQ ID NO:84.

In some embodiments, an sBCMA variant—Fc fusion protein has the amino acid sequence as set forth in SEQ ID NO:80.

In some embodiments, an sBCMA variant—Fc fusion protein has the amino acid sequence as set forth in SEQ ID NO:81.

In some embodiments, an sBCMA variant—Fc fusion protein has the amino acid sequence as set forth in SEQ ID NO:82.

In some embodiments, an sBCMA variant—Fc fusion protein has the amino acid sequence as set forth in SEQ ID NO:83.

In some embodiments, an sBCMA variant—Fc fusion protein has the amino acid sequence as set forth in SEQ ID NO:84.

### E. Nucleic Acids

The present disclosure also provides compositions including nucleic acids encoding a variant sBCMA and/or sBCMA variant—Fc fusion protein. Such nucleic acids encode any of the variant sBCMA and/or sBCMA variant—Fc fusion proteins recited herein.

Nucleic acids may be isolated and/purified. In various embodiments, nucleic acids, either as DNA or RNA, are substantially free of other naturally occurring nucleic acid sequences. In some embodiments, nucleic acids are at least about 50%, or at least about 90% pure. In a number of embodiments, nucleic acids are “recombinant,” i.e., flanked by one or more nucleotides with which it is not normally associated on a naturally occurring chromosome.

In some embodiments, a composition includes a nucleic acid encoding any variant sBCMA as described herein.

In some embodiments, a composition includes a nucleic acid encoding any sBCMA variant—Fc fusion protein as described herein.

In some embodiments, a nucleic acid encodes a BCMA variant-Fc fusion protein including a signal sequence. As is known in the art, signal sequences encode a short peptide that, when linked, directs a protein (or peptide) through the secretory pathway, often resulting in the protein being excreted from the cell. As will be appreciated by those in the art, suitable signal sequences for expression of the fusion proteins can be selected as appropriate for the host cell. That is, when the fusion proteins of the invention are to be expressed in mammalian host cells such as CHO cells, for example, signal sequences from CHO cells can be used.

In some embodiments, a composition includes a nucleic acid encoding the BCMA—Fc fusion protein as described herein.

In some embodiments, a composition includes a nucleic acid encoding the BCMA—Fc fusion protein exhibiting at least 90%, 95%, 96%, 97%, 98% or 99% identity to SEQ ID NO:80.

In some embodiments, a composition includes a nucleic acid encoding the BCMA—Fc fusion protein exhibiting at least 90%, 95%, 96%, 97%, 98% or 99% identity to SEQ ID NO:81.

In some embodiments, a composition includes a nucleic acid encoding the BCMA—Fc fusion protein exhibiting at least 90%, 95%, 96%, 97%, 98% or 99% identity to SEQ ID NO:82.

In some embodiments, a composition includes a nucleic acid encoding the BCMA—Fc fusion protein exhibiting at least 90%, 95%, 96%, 97%, 98% or 99% identity to SEQ ID NO:83.

In some embodiments, a composition includes a nucleic acid encoding the BCMA—Fc fusion protein exhibiting at least 90%, 95%, 96%, 97%, 98% or 99% identity to SEQ ID NO:84.

In some embodiments, a composition includes a nucleic acid encoding the BCMA—Fc fusion protein having the amino acid sequence of SEQ ID NO:80.

In some embodiments, a composition includes a nucleic acid encoding the BCMA—Fc fusion protein having the amino acid sequence of SEQ ID NO:81.

In some embodiments, a composition includes a nucleic acid encoding the BCMA—Fc fusion protein having the amino acid sequence of SEQ ID NO:82.

In some embodiments, a composition includes a nucleic acid encoding the BCMA-Fc fusion protein having the amino acid sequence of SEQ ID NO:83.

In some embodiments, a composition includes a nucleic acid encoding the BCMA-Fc fusion protein having the amino acid sequence of SEQ ID NO:84.

In some embodiments, a BCMA variant—Fc fusion protein encoding nucleic acid includes a codon optimized version or variant.

“Codon optimized” in this context is done in relation to a particular host organism and its generally preferred amino acid codons; that is, the host production organism, e.g. an Aspergillus species, may yield higher translation and/or secretion using Aspergillus preferred codons as compared to a yeast production organism.

Codon optimization can be employed with any of the sBCMA (variant)—Fc fusion protein, which may yield higher expression in the host cell employed.

An sBCMA (variant)—Fc fusion protein can be prepared generally by nucleic acid sequences encoding the fusion protein sequence using well known techniques, including site-directed mutagenesis of a parental gene and synthetic gene recombination techniques.

Expression nucleic acids can be regulated by their own or by other regulatory sequences known in the art. In various embodiments, expression is regulated by incorporating specific regulatory sequences to yield a desired expression effect, as understood in the art.

### 1. Regulatory Sequences

The present invention also relates to nucleic acid constructs comprising a polynucleotide encoding a variant sBCMA or an sBCMA variant—Fc fusion protein operably linked to one or more regulatory sequences that direct the expression of the coding sequence in a suitable host cell under conditions compatible with the control sequences. Regulatory sequences may include a promoter. A promoter contains transcriptional control sequences that mediate the expression levels of a protein, such as a variant protein, and a fusion protein as described herein. A promoter may be any polynucleotide that shows transcriptional activity in a host cell, including mutant, truncated, and hybrid promoters, and may be obtained from genes encoding extracellular or intracellular polypeptides either homologous or heterologous to the host cell.

### F. Expression Vectors

Also provided herein are expression vectors for in vitro or in vivo expression of one or more variant sBCMA and sBCMA variant—Fc fusion proteins, either constitutively or under one or more regulatory elements. In some embodiments, expression vectors include a polynucleotide encoding the variant sBCMA or sBCMA variant—Fc fusion protein, a promoter, and transcriptional and translational stop signals. Various nucleotide and control sequences may be joined together to produce a recombinant expression vector that may include one or more convenient restriction sites to allow for insertion or substitution of the polynucleotide encoding the Fc fusion protein at such sites. Alternatively, the polynucleotide may be expressed by inserting the polynucleotide or a nucleic acid construct comprising the polynucleotide into an appropriate vector for expression. In creating an expression vector, the coding sequence is located in the vector so that the coding sequence is operably linked with the appropriate control sequences for expression.

A recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector can be a linear or closed circular plasmid.

A vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. A vector may contain any means for assuring self-replication. Alternatively, a vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used. Vectors contemplated include both integrating and non-integrating vectors.

### G. Host Cells and Production Strains

As will be appreciated by those in the art, there are a wide variety of production host organisms for recombinant production of variant sBCMA proteins and sBCMA variant—Fc fusion proteins as described herein, including, but not limited to bacterial cells, mammalian cells and fungal cells including yeast.

The nucleic acids of the invention can be introduced into suitable host cells using a variety of techniques available in the art, such as transferrin polycation-mediated DNA transfer, transfection with naked or encapsulated nucleic acids, liposome-mediated DNA transfer, intracellular transportation of DNA-coated latex beads, protoplast fusion, viral infection, electroporation, gene gun, calcium phosphate-mediated transfection, and the like.

### H. Methods of Making the Fusion Proteins

The present invention also relates to methods of making a variant sBCMA, comprising: (a) cultivating a host cell of the present invention under conditions suitable for expression of the variant sBCMA; and (b) optionally recovering the variant sBCMA.

The present invention also relates to methods of making an sBCMA variant—Fc fusion protein, comprising: (a) cultivating a host cell of the present invention under conditions suitable for expression of the sBCMA variant—Fc fusion protein; and (b) optionally recovering the sBCMA variant—Fc fusion protein.

### I. Methods of Treatment

### 1. Subjects Amenable to Treatment

Various embodiments are directed to therapeutic methods, many of which include administering to a subject in need of treatment a therapeutically effective amount of one or more variant sBCMA proteins as described herein.

Various embodiments are directed to therapeutic methods, many of which include administering to a subject in need of treatment a therapeutically effective amount of one or more sBCMA variant—Fc fusion proteins as described herein.

In some embodiments, the present invention provides a method of treating one or more B-cell malignancies in a subject, wherein the method comprises administering to the subject a therapeutically effective dose of one or more said variant sBCMA proteins as described above. In some embodiments, the one or more B-cell malignancies are selected from the group consisting of multiple myeloma, diffuse large B-cell lymphoma, primary mediastinal B-cell lymphoma, follicular lymphoma, chronic lymphocytic leukemia, mantle cell lymphoma, extranodal marginal zone B-cell lymphoma—mucosa-associated lymphoid tissue (MALT) lymphoma, nodal marginal zone B-cell lymphoma, splenic marginal zone B-cell lymphoma, Burkitt lymphoma, lymphoplasmacytic lymphoma (Waldenstrom macroglobulinemia), hairy cell leukemia, primary central nervous system (CNS) lymphoma, and primary intraocular lymphoma (lymphoma of the eye). In some embodiments, the subject is a human subject.

In some embodiments, the present invention provides a method of treating one or more B-cell malignancies in a subject, wherein the method comprises administering to the subject a therapeutically effective dose of one or more said sBCMA variant—Fc fusion proteins as described above. In some embodiments, the one or more B-cell malignancies are selected from the group consisting of multiple myeloma, diffuse large B-cell lymphoma, primary mediastinal B-cell lymphoma, follicular lymphoma, chronic lymphocytic leukemia, mantle cell lymphoma, extranodal marginal zone B-cell lymphoma—mucosa-associated lymphoid tissue (MALT) lymphoma, nodal marginal zone B-cell lymphoma, splenic marginal zone B-cell lymphoma, Burkitt lymphoma, lymphoplasmacytic lymphoma (Waldenstrom macroglobulinemia), hairy cell leukemia, primary central nervous system (CNS) lymphoma, and primary intraocular lymphoma (lymphoma of the eye). In some embodiments, the subject is a human subject.

A number of embodiments are directed to a method of treating, reducing or preventing metastasis or invasion of a tumor that expresses APRIL in a subject, wherein the method comprises administering to the subject a therapeutically effective dose of one or more said variant sBCMA proteins as described above. In some embodiments, the tumor as disclosed herein is the B-cell malignancy as disclosed above. In some embodiments, the tumor as disclosed herein is a hematologic cancer. In some embodiments, the hematologic cancer as disclosed herein is multiple myeloma.

A number of embodiments are directed to a method of treating, reducing or preventing metastasis or invasion of a tumor that expresses APRIL in a subject, where the method comprises administering to the subject a therapeutically effective dose of one or more said sBCMA variant—Fc fusion proteins as described above. In some embodiments, the tumor as disclosed herein is the B-cell malignancy as disclosed above. In some embodiments, the tumor as disclosed herein is a hematologic cancer. In some embodiments, the hematologic cancer as disclosed herein is multiple myeloma.

A number of embodiments are directed to a method of treating, reducing or preventing metastasis or invasion of a tumor that expresses BCMA, TACI and/or other receptors that are activated through binding to APRIL in a subject, where the method comprises administering to the subject a therapeutically effective dose of one or more said variant sBCMA proteins as described above. In some embodiments, the tumor as disclosed herein is the B-cell malignancy as disclosed above. In some embodiments, the tumor as disclosed herein is a hematologic cancer. In some embodiments, the hematologic cancer as disclosed herein is multiple myeloma.

A number of embodiments are directed to a method of treating, reducing or preventing metastasis or invasion of a tumor that expresses BCMA, TACI and/or other receptors that are activated through binding to APRIL in a subject, where the method comprises administering to the subject a therapeutically effective dose of one or more said sBCMA variant—Fc fusion proteins as described above. In some embodiments, the tumor as disclosed herein is the B-cell malignancy as disclosed above. In some embodiments, the tumor as disclosed herein is a hematologic cancer. In some embodiments, the hematologic cancer as disclosed herein is multiple myeloma.

Some embodiments are directed to a method of inhibiting the activity of APRIL in a subject having a tumor that expresses APRIL, where the method comprises administering to the subject a therapeutically effective dose of one or more said variant sBCMA proteins as described above. In some embodiments, the tumor as disclosed herein is the B-cell malignancy as disclosed above. In some embodiments, the tumor as disclosed herein is a hematologic cancer. In some embodiments, the hematologic cancer as disclosed herein is multiple myeloma.

Some embodiments are directed to a method of inhibiting the activity of APRIL in a subject having a tumor that expresses APRIL, where the method comprises administering to the subject a therapeutically effective dose of one or more said sBCMA variant—Fc fusion proteins as described above. In some embodiments, the tumor as disclosed herein is the B-cell malignancy as disclosed above. In some embodiments, the tumor as disclosed herein is a hematologic cancer. In some embodiments, the hematologic cancer as disclosed herein is multiple myeloma.

Some embodiments are directed to a method of inhibiting the activity of APRIL in a subject having a tumor that expresses BCMA, TACI and/or other receptors that are activated through binding to APRIL, where the method comprises administering to the subject a therapeutically effective dose of one or more said variant sBCMA proteins as described above. In some embodiments, the tumor as disclosed herein is the B-cell malignancy as disclosed above. In some embodiments, the tumor as disclosed herein is a hematologic cancer. In some embodiments, the hematologic cancer as disclosed herein is multiple myeloma.

Some embodiments are directed to a method of inhibiting the activity of APRIL in a subject having a tumor that expresses BCMA, TACI and/or other receptors that are activated through binding to APRIL, where the method comprises administering to the subject a therapeutically effective dose of one or more said sBCMA variant—Fc fusion proteins as described above. In some embodiments, the tumor as disclosed herein is the B-cell malignancy as disclosed above. In some embodiments, the tumor as disclosed herein is a hematologic cancer. In some embodiments, the hematologic cancer as disclosed herein is multiple myeloma.

Some embodiments are directed to a method of inhibiting the activity of APRIL in a subject having an autoimmune disease that expresses APRIL, where the method comprises administering to the subject a therapeutically effective dose of one or more said variant sBCMA proteins as described above.

Some embodiments are directed to a method of inhibiting the activity of APRIL in a subject having an autoimmune disease that expresses APRIL, where the method comprises administering to the subject a therapeutically effective dose of one or more said sBCMA variant—Fc fusion proteins as described above.

Some embodiments are directed to a method of inhibiting the activity of APRIL in a subject having an autoimmune disease that expresses BCMA, TACI and/or other receptors that are activated through binding to APRIL, where the method comprises administering to the subject a therapeutically effective dose of one or more said variant sBCMA proteins as described above.

Some embodiments are directed to a method of inhibiting the activity of APRIL in a subject having an autoimmune disease that expresses BCMA, TACI and/or other receptors that are activated through binding to APRIL, where the method comprises administering to the subject a therapeutically effective dose of one or more said sBCMA variant—Fc fusion proteins as described above.

Some embodiments are directed to a method of inhibiting the activity of APRIL in a subject having fibrosis that expresses APRIL, where the method comprises administering to the subject a therapeutically effective dose of one or more said variant sBCMA proteins as described above.

Some embodiments are directed to a method of inhibiting the activity of APRIL in a subject having fibrosis that expresses APRIL, where the method comprises administering to the subject a therapeutically effective dose of one or more said sBCMA variant—Fc fusion proteins as described above.

Some embodiments are directed to a method of inhibiting the activity of APRIL in a subject having fibrosis that expresses BCMA, TACI and/or other receptors that are activated through binding to APRIL, where the method comprises administering to the subject a therapeutically effective dose of one or more said variant sBCMA proteins as described above.

Some embodiments are directed to a method of inhibiting the activity of APRIL in a subject having fibrosis that expresses BCMA, TACI and/or other receptors that are activated through binding to APRIL, where the method comprises administering to the subject a therapeutically effective dose of one or more said sBCMA variant—Fc fusion proteins as described above.

Some embodiments are directed to a method of inhibiting B-cell growth, immunoglobulin production, or both in a subject, where the method comprises administering to the subject a therapeutically effective dose of one or more said variant sBCMA proteins as described above, and where the variant sBCMA protein binds to BAFF.

Some embodiments are directed to a method of inhibiting B-cell growth, immunoglobulin production, or both in a subject, where the method comprises administering to the subject a therapeutically effective dose of one or more said sBCMA variant—Fc fusion proteins as described above, and where the sBCMA domain binds to BAFF.

Some embodiments are directed to a method of inhibiting the activity of BAFF in a subject having B cell hyperplasia or an autoimmune disease expressing BCMA, BAFFR, TACI and/or other receptors that are activated through binding to BAFF, where the method comprises administering to the subject a therapeutically effective dose of one or more said variant sBCMA proteins as described above.

Some embodiments are directed to a method of inhibiting the activity of BAFF in a subject having B cell hyperplasia or an autoimmune disease expressing BCMA, BAFFR, TACI and/or other receptors that are activated through binding to BAFF, where the method comprises administering to the subject a therapeutically effective dose of one or more said sBCMA variant—Fc fusion proteins as described above.

Some embodiments are directed to a method of treating an autoimmune disease expressing at least one receptor selected from the group consisting of BCMA, BAFFR, TACI and other receptor(s) that are activated through binding to BAFF in a subject, said method comprising administering to the subject a therapeutically effective dose of one or more said variant sBCMA proteins as described above.

Some embodiments are directed to a method of treating an autoimmune disease expressing at least one receptor selected from the group consisting of BCMA, BAFFR, TACI and other receptor(s) that are activated through binding to BAFF in a subject, said method comprising administering to the subject a therapeutically effective dose of one or more said sBCMA variant—Fc fusion proteins as described above.

Some embodiments are directed to a method of treating an autoimmune disease expressing BAFF and/or APRIL in a subject, said method comprising administering to the subject a therapeutically effective dose of one or more said variant sBCMA proteins as described above.

Some embodiments are directed to a method of treating an autoimmune disease expressing BAFF and/or APRIL in a subject, said method comprising administering to the subject a therapeutically effective dose of one or more said sBCMA variant—Fc fusion proteins as described above.

Some embodiments are directed to a method of treating fibrosis expressing BCMA, BAFFR and/or TACI in a subject, said method comprising administering to the subject a therapeutically effective dose of one or more said variant sBCMA proteins as described above.

Some embodiments are directed to a method of treating fibrosis expressing BCMA, BAFFR and/or TACI in a subject, said method comprising administering to the subject a therapeutically effective dose of one or more said sBCMA variant—Fc fusion proteins as described above.

Some embodiments are directed to a method of treating fibrosis expressing BAFF and/or APRIL in a subject, said method comprising administering to the subject a therapeutically effective dose of one or more said variant sBCMA proteins as described above.

Some embodiments are directed to a method of treating fibrosis expressing BAFF and/or APRIL in a subject, said method comprising administering to the subject a therapeutically effective dose of one or more said sBCMA variant—Fc fusion proteins as described above.

### 2. Therapeutic Administration

In certain embodiments, a therapeutically effective composition or formulation having one or more variant sBCMA proteins may be administered systemically to the individual or via any other route of administration known in the art.

In certain embodiments, a therapeutically effective composition or formulation having one or more sBCMA variant—Fc fusion proteins may be administered systemically to the individual or via any other route of administration known in the art.

### 3. Dosing

In some embodiments, an effective dose of the therapeutic entity of the present invention, e.g. for the treatment of cancers or immunomodulatory disorders, varies depending upon many different factors, including means of administration, target site, physiological state of the patient, whether the patient is human or an animal, other medications administered, and whether treatment is prophylactic or therapeutic. Treatment dosages can be titrated to optimize safety and efficacy.

### VI. EXAMPLES

### A. Example 1: Synthesis of Yeast-Displayed sBCMA Library

DNA encoding human BCMA extracellular domain, amino acids Met1-Ala54, was cloned into the pCT yeast display plasmid using NheI and BamHI restriction sites. Sequence numbering was done to match that used in Sasaki et al. to facilitate comparisons to their work with the wild-type proteins. An error-prone library was created using the BCMA extracellular domain DNA as a template and mutations were introduced by using low-fidelity Taq polymerase (Invitrogen) and the nucleotide analogs 8-oxo-dGTP and dPTP (TriLink Biotech). Six separate PCR reactions were performed in which the concentration of analogs and the number of cycles were varied to obtain a range of mutation frequencies; five cycles (200 μM), ten cycles (2, 20, or 200 μM), and 20 cycles (2 or 20 μM). Products from these reactions were amplified using forward and reverse primers each with 50 bp homology to the pCT plasmid in the absence of nucleotide analogs. Amplified DNA was purified using gel electrophoresis and pCT plasmid was digested with NheI and BamHI. Purified mutant cDNA and linearized plasmid were electroporated in a 5:1 ratio by weight into EBY100 yeast where they were assembled in vivo through homologous recombination. Library size was estimated to be 2×10⁸ by dilution plating.

### B. Example 2: Library Screening

Yeast displaying high affinity BCMA mutants were isolated from the library using fluorescence-activated cell sorting (FACS). For FACS round 1, equilibrium binding sorts were performed in which yeast were incubated at room temperature in phosphate buffered saline with 0.1% BSA (PBSA) with the 2 nM APRIL (Peprotech) for 24 h. After incubation with APRIL, yeast were pelleted, washed, and resuspended in PBSA with 1:100 mixture of anti-c-Myc FITC antibody (Abcam) and anti-HA AF647 (Invitrogen) for 1 h at 4° C. Yeast were then washed, pelleted and resuspended using PBSA followed by FACS analysis.

For FACS rounds 2-6, kinetic off-rate sorts were conducted in which yeast were incubated with 2 nM APRIL for 3 hours at room temperature, after which cells were washed twice to remove excess unbound APRIL, and resuspended in PBSA containing a ˜50 fold molar excess of BCMA to render unbinding events irreversible. The length of the unbinding step was as follows: sort 2) 48 h; sort 3, 4 & 5) 72 h; sort 6) 84 h, with all unbinding reactions performed at room temperature. During the last hour of the dissociation reaction, cells were mixed with 1:100 mixture of anti-c-Myc FITC antibody (Abcam) and anti-HA AF647 (Invitrogen) for 1 h at 4° C. Yeast were pelleted, washed, and resuspended in 0.1% BSA. Labeled yeast were sorted by FACS using a Vantage SE flow cytometer (Stanford FACS Core Facility) and CellQuest software (Becton Dickinson). Sorts were conducted such that the 1-3% of clones with the highest APRIL binding/c-Myc expression ratio were selected, enriching the library for clones with the highest binding affinity to APRIL. In sort 1, 10⁸ cells were screened and subsequent rounds analyzed a minimum of ten-fold the number of clones collected in the prior sort round to ensure adequate sampling of the library diversity. Selected clones were propagated and subjected to further rounds of FACS. Following sorts 3, 4, 5 and 6, plasmid DNA was recovered using a Zymoprep kit (Zymo Research Corp.), transformed into DH5a supercompetent cells, and isolated using plasmid miniprep kit (Qiagen). Sequencing was performed by MCLAB.

Analysis of yeast-displayed sort products was performed using the same reagents and protocols and described for the library sorts. Samples were analyzed on a FACS Calibur (BD Biosciences) and data was analyzed using FlowJo software (Treestar Inc.).

### C. Example 3: Binding Affinity Assay

Cells were cultured in standard tissue culture condition. Cells were harvested and the supernatant discarded then dispensed onto a staining plate at 3×10⁵ cells per well. The plate was centrifuged at 300 g at 4° C. for 5 minutes. Various concentrations of sBCMA mutants and negative control were diluted in FACS buffer containing 2% FBS, 100 μL/well was added. Cells were incubated for 1 hour at 4° C. and washed twice with 200 μL FACS buffer and centrifuged at 300 g for 5 minutes. The supernatant was discarded before and after each wash. Cells were re-suspended at 100 μL/well with 1:1000 diluent with anti-human IgG-Alexa 488 (#A28175, ThermoFisher, Waltham, MA). Plates were incubated for 1 hour at 4° C. Cells were washed twice with FACS buffer and centrifuged at 300 g for 5 minutes. Supernatant was discarded and cells were re-suspended in 100 μL cold PBS. The cells were kept in the dark and FACS analysis carried out on FACS CantoII, (BD Biosciences, San Jose, CA). Geometric mean (measure of binding affinity) of double positive population was determined by using FlowJo software. In order to determine the Kd (ligand concentration that binds to half the receptor sites at equilibrium) of the binding reaction, binding affinity was plotted against ligand concentration and the graph was analyzed using one site-specific binding in Graphpad Prism to get the Kd value.

### D. Example 4: Results

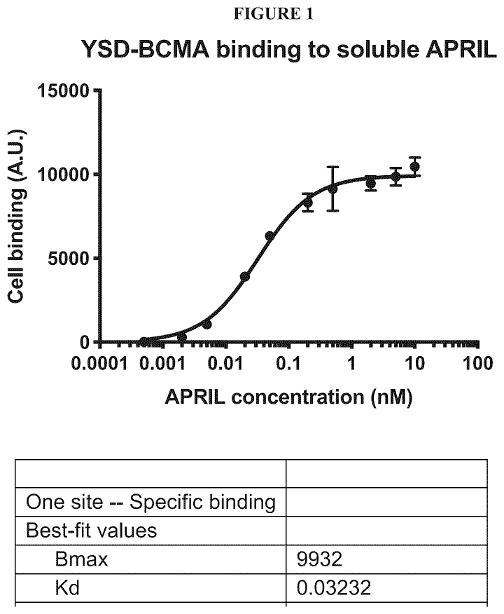

The binding kinetics of WT-BCMA binding to APRIL is showed in FIG. 1. The binding assay for WT-BCMA binding to APRIL shows a Kd of 32 μM.

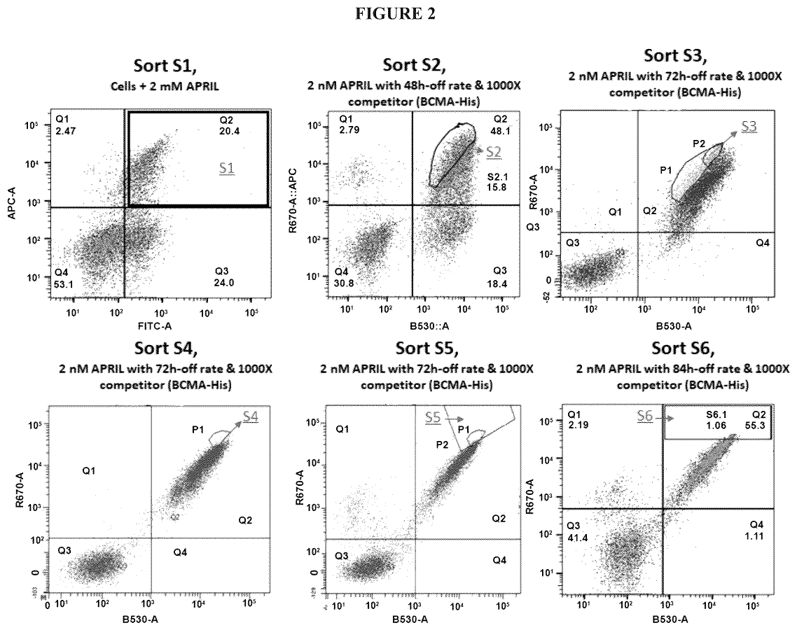

Various sorting conditions and gates for BCMA yeast display library are shown in FIG. 2.

FIG. 3 shows sequencing variant sBCMA clones to determine mutations as compared to wild type sBCMA.

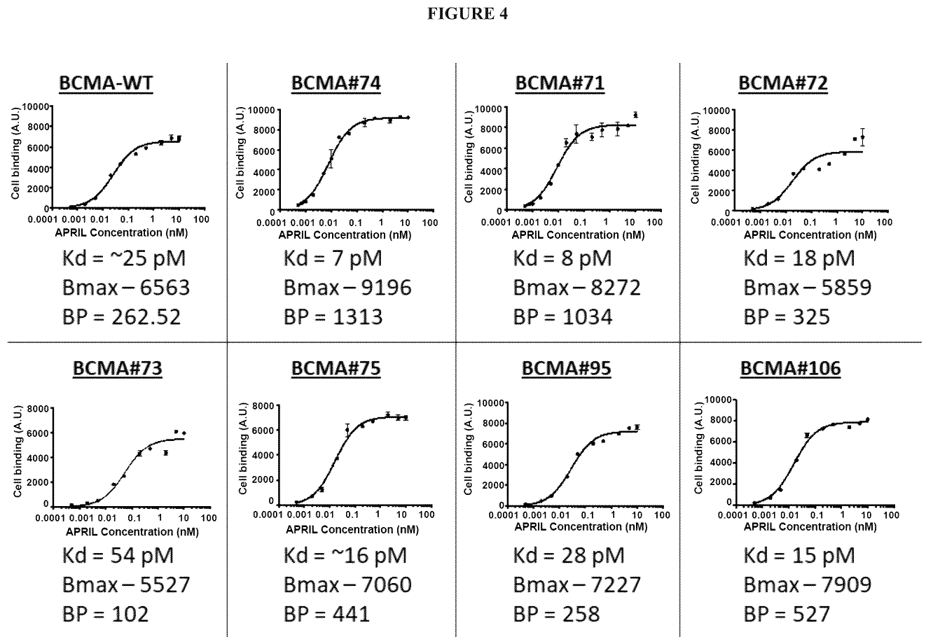

The binding curves and binding results for various variant sBCMA clones to APRIL are shown in FIG. 4.

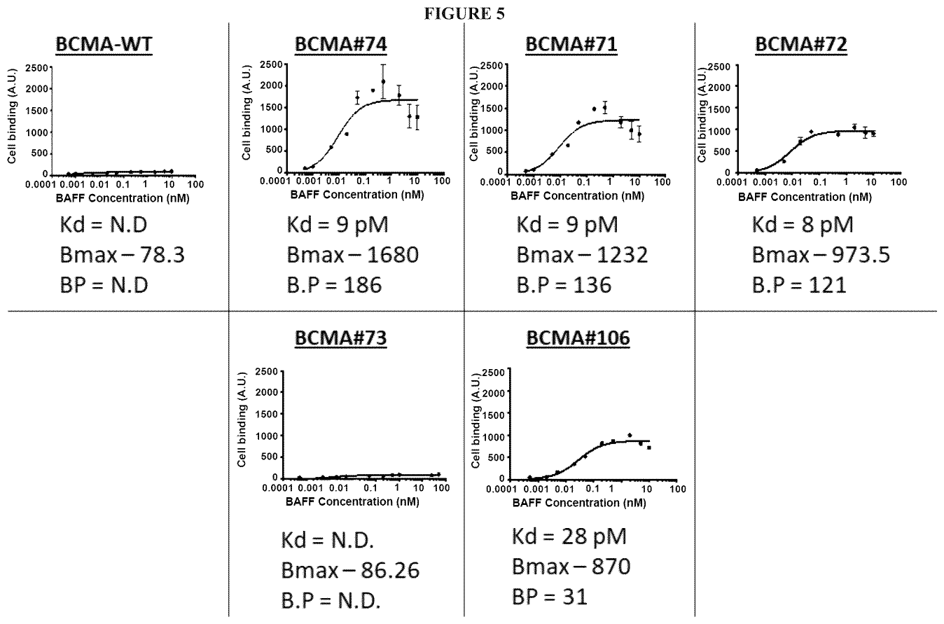

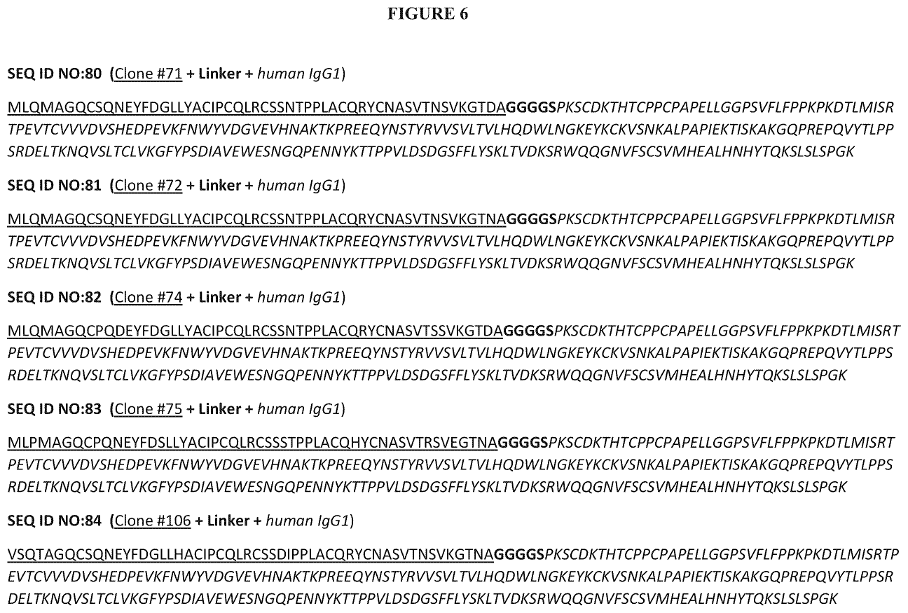

The binding curves and binding results for various variant sBCMA clones to BAFF are shown in FIG. 5.

### E. Example 5: In Vivo Tumor Efficacy Study

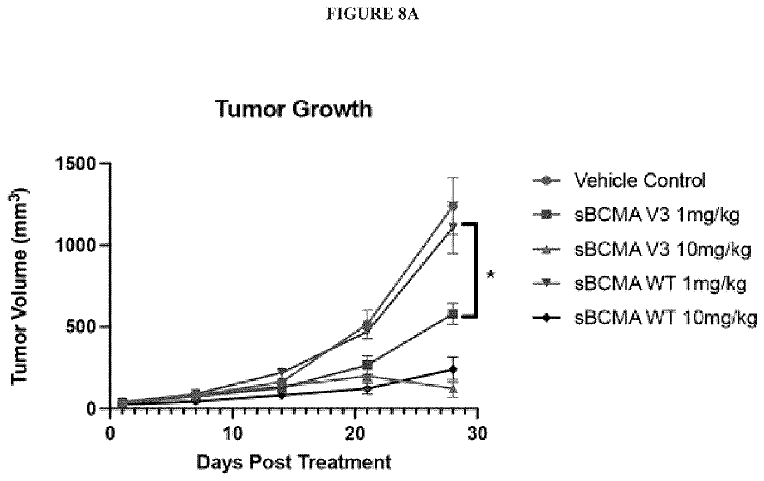

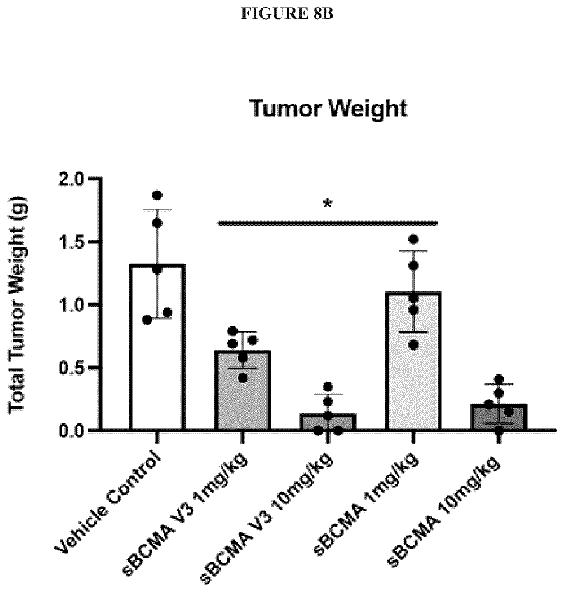

Anti-tumor activity of sBCMA variant—Fc fusion protein (hereinafter “variant sBCMA-Fc”) administered at 1 mg/kg and 10 mg/kg was analyzed and compared to the anti-tumor activity of wild type sBCMA—Fc fusion protein (hereinafter “wild type sBCMA-Fc”) administered at 1 mg/kg and 10 mg/kg. MM1.R Multiple Myeloma Cells were injected subcutaneously into NSG immunocompromised mice. Animals were treated with vehicle control, 1 mg/kg variant sBCMA-Fc, 10 mg/kg variant sBCMA-Fc, 1 mg/kg wild type sBCMA-Fc and 10 mg/kg wild type sBCMA-Fc. Tumor growths were compared between control and treated groups throughout experiments. Final tumor weight of harvested MM1.R Multiple Myeloma tumors were compared among control and treated groups. FIG. 8 shows results of tumor growth curves (FIG. 8A) and terminal tumor weights (FIG. 8B). This study showed significantly enhanced anti-tumor activity in animals treated with 1 mg/kg variant sBCMA-Fc, 10 mg/kg variant sBCMA-Fc and 10 mg/kg wild type sBCMA-Fc compared to vehicle treated animals. Specifically, animals treated with 1 mg/kg variant sBCMA-Fc showed significantly improved anti-tumor activity compared to mice treated with 1 mg/kg wild type sBCMA-Fc.

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains. All references cited in this disclosure are incorporated by reference to the same extent as if each reference had been incorporated by reference in its entirety individually.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only.

1. A composition comprising a variant soluble B-cell maturation antigen (sBCMA) comprising at least one amino acid substitution as compared to SEQ ID NO:1, wherein said amino acid substitution is at a position number selected from the group consisting of 1, 2, 3, 4, 5, 6, 7, 9, 10, 11, 12, 14, 16, 19, 20, 22, 23, 25, 26, 29, 31, 32, 35, 36, 38, 39, 42, 43, 44, 45, 46, 47, 48, 49, 50, 51, 52, 53, and 54, wherein the numbering is according to the EU index.

2. The composition according to claim 1, wherein said variant sBCMA has at least 80%, at least 85%, at least 90%, or at least 95% sequence identity to SEQ ID NO:1.

3. The composition according to any one of the preceding claims, wherein said amino acid substitution(s) occur at one of said positions, two of said positions, three of said positions, four of said positions, five of said positions, six of said positions, seven of said positions, eight of said positions, or nine of said positions.

4. The composition according to any one of the preceding claims, wherein said amino acid substitution(s) is selected from the group consisting of M1A, M1C, M1I, M1R, M1T, M1V, L2C, L2S, Q3P, Q3R, M4E, M4I, M4T, M4V, A5T, G6E, Q7R, S9A, S9F, S9P, Q10H, Q10P, Q10R, N11D, N11S, E12K, F14L, S16G, S16N, S16R, H19L, H19Y, A20V, A20T, I22M, I22V, P23S, Q25R, L26F, S29A, N31D, N31S, T32A, T32I, T32P, L35S, L35P, T36A, T36I, T36P, Q38R, R39H, N42D, N42R, N42S, A43T, A43V, S44D, S44G, S44N, S44R, V45A, V45M, T46A, T46I, N47D, N47K, N47R, N47S, S48L, S48P, S48T, V49A, V49M, K50E, K50G, K50R, K50T, G51E, T52A, T52M, N53D, N53K, N53S, A54V, and A54T.

5. The composition according to any one of the preceding claims, wherein said amino acid substitution(s) is selected from the group consisting of M1V, L2S, Q3P, M4T, S9P, N11D, S16G, H19Y, N31S, N31 D, T32I, T36A, R39H, N47S, K50E, and N53E.

6. The composition according to any one of the preceding claims, wherein said amino acid substitution(s) is selected from the group consisting of S16G, H19Y and T36A.

7. The composition according to any one of claims 1-4, wherein said amino acid substitutions are selected from the group consisting of L2S/S9P/E12K/N31D/T36A/N42S/N53S, M1V/T32P/T36A/T46I/N53D/A54V, Q3R/S16N/T36A/A43T, F14L/S16G/T36A/V45A/N47D, M1T/M4V/S9F/S16G/T32A/Q38R, M1A/S9A/Q38R, G6E/Q25R/Q38R, M1V/M4I/G6E/S9P/N11D/V49M/T52M/A54V, N11D/S16G/N31 S, N11D/H19Y/I22M/T32P/N47S/N53S, G6E/Q7R/H19Y/L35S, H19Y/N42D/S48P/T52A, M1V/N31D/T32I/T36A, M1V/A5T/H19L/T36A, M1T/N31D/T32A/T36A/Q38R/S44D/V49A/K50E, M1V/T36A/Q38R/A43V, M1V/L2S/S9P/Q10H/T36A/Q38R/K50G, T36A/Q38R/N53S, M1T/L2S/L35P/T36A/Q38R/T46A/K50R, A5T/A20V/T36A/Q38R, M1T/S16G/I22V/T36A/S44G/T46A/V49A, S16G/T36A, M1I/N11D/S16G/I22M/S29A/T36A/S44G/K50R, M1C/L2C/Q3R/M4E/N11D/S16G/T36P, M1I/N11D/S16G/I22M/S29A/T36A/S44G/K50R, N11D/N31D/T32I/T36A/S44N/N47D/N53D, M1R/L2C/Q3R, H19Y/T36A/S44G, H19Y/T32I/T36A/V49A, H19Y/N31 S/T36A/V45A, H19Y/N31S/T36A, H19Y/T36P/T52A, H19Y/N31D/T52M, M1V/H19Y/V45M, S16G/H19Y/N47D, S16G/H19Y/K50T, S16G/H19Y/S44N/K50R, N11D/H19Y/S48T, S9P/N11D/S16R/T32A/Q38R/S44G/T46I/T52A/N53D/A54T, N11D/S16G/S44R, H19L/T32A/S44G/G51E/T52A, S16N/H19Y/T36A/K50R, M1V/H19Y/T36A/R39H/T46A, M1V/H19Y/T36A, H19Y/T36A/N42D/N47S/S48P, M1V/H19Y/T36A/S44G/N47D, M1V/H19Y/T36A/N42R/N53S, H19Y/L35P/T36A/N42D/T46l/V49A, Q3P/S9P/H19Y/N31 S/T36A/R39H/N47R/K50E, M1V/H19Y/T36A/N42R/N53S, M1T/H19Y/T36A, M1V/S16N/H19Y/I22M/T36A, M1T/N11D/H19Y/T36A/N42S/V45A/N53S, N11D/S16G/H19Y/T36A/N47S/N53D, M1V/S9P/Q10P/S16G/H19Y/L26F/T36A/A43V/N53D, S16G/H19Y/T36A/V49A/N53D, S16G/T36A/A43T/S44G/V45M, M4V/S9P/S16G/T36A/Q38R, S9P/N11S/S16G/T36A/Q38R, N11D/E12K/S16R/T36A/T52M, M4V/T32I/T36A/Q38R/A43T/V45A/S48P, S9P/N11D/S16G/Q25R, M1T/A5T/S9P/S16G/Q25R/N31D/V49M, L2S/S9P/S16G/A20T/T32I/Q38R/N42D/T46A/S48L, S16G/Q25R/T46A, G6E/S9A/S16G/Q25R/N31D/N47S/T52M, H19Y/Q38R/T52M, N11D/H19Y/I22M/T32P/N47S/N53S, S16G/H19Y/T36A, S16G/H19Y/T36A/N53D, S9P/N11D/S16G/H19Y/T36A/N47S/N53D, Q3P/S9P/H19Y/N31 S/T36A/R39H/N47R/K50E, M1V/L2S/M4T/N11D/H19Y/T36A, M1V/L2S/M4T/N11D/T36A, M1V/L2S/M4T/H19Y/T36I/V45A/V49M, M1V/L2S/M4T/N11D/H19Y/T36A, M1V/L2S/M4T/S9P/Q10R/H19Y/T36A/T46A/N47S, M1V/L2S/M4T/S16G/N31D/T32I/T36A, M1V/M4T/T36A/Q38R/N53K, M1T/N11D/H19Y/T36A/N42S/V45A/N53S, M1T/N31D/T32A/T36A/A38R/S44D/V49A/K50E, M1T/S9P/P23S/Q38R/N42S/S48P/V49A/A54V, H19Y/T36A/S44G, H19Y/T36A, and M4T/T36A/Q38R/N42S/S44G/T46A/N47K/S48P/T52A.

8. The composition according to any one of claims 1-4 and 7, wherein said variant sBCMA comprises the amino acid substitutions S16G/H19Y/T36A, and at least one further amino acid substitution selected from the group consisting of M1A, M1C, M1I, M1R, M1T, M1V, L2C, L2S, Q3P, Q3R, M4E, M4I, M4T, M4V, A5T, G6E, Q7R, S9A, S9F, S9P, Q10H, Q10P, Q10R, N11D, N11S, E12K, F14L, S16N, S16R, H19L, A20V, A20T, I22M, I22V, P23S, Q25R, L26F, S29A, N31D, N31S, T32A, T32I, T32P, L35S, L35P, T36I, T36P, Q38R, R39H, N42D, N42R, N42S, A43T, A43V, S44D, S44G, S44N, S44R, V45A, V45M, T46A, T46I, N47D, N47K, N47R, N47S, S48L, S48P, S48T, V49A, V49M, K50E, K50G, K50R, K50T, G51E, T52A, T52M, N53D, N53K, N53S, A54V and A54T.

9. The composition according to any one of claims 1-4 and 7, wherein said variant sBCMA comprises the amino acid substitutions S16G/H19Y/T36A/N53D, and at least one further amino acid substitution selected from the group consisting of M1A, M1C, M1I, M1R, M1T, M1V, L2C, L2S, Q3P, Q3R, M4E, M4I, M4T, M4V, A5T, G6E, Q7R, S9A, S9F, S9P, Q10H, Q10P, Q10R, N11D, N11S, E12K, F14L, S16N, S16R, H19L, A20V, A20T, I22M, I22V, P23S, Q25R, L26F, S29A, N31D, N31S, T32A, T32I, T32P, L35S, L35P, T36I, T36P, Q38R, R39H, N42D, N42R, N42S, A43T, A43V, S44D, S44G, S44N, S44R, V45A, V45M, T46A, T46I, N47D, N47K, N47R, N47S, S48L, S48P, S48T, V49A, V49M, K50E, K50G, K50R, K50T, G51E, T52A, T52M, N53K, N53S, A54V, and A54T.

10. The composition according to any one of claims 1-4 and 7, wherein said variant sBCMA comprises the amino acid substitutions S9P/N11D/S16G/H19Y/T36A/N47S/N53D, and at least one further amino acid substitution selected from the group consisting of M1A, M1C, M1I, M1R, M1T, M1V, L2C, L2S, Q3P, Q3R, M4E, M4I, M4T, M4V, A5T, G6E, Q7R, S9A, S9F, Q10H, Q10P, Q10R, N11S, E12K, F14L, S16N, S16R, H19L, A20V, A20T, I22M, I22V, P23S, Q25R, L26F, S29A, N31D, N31S, T32A, T32I, T32P, L35S, L35P, T36I, T36P, Q38R, R39H, N42D, N42R, N42S, A43T, A43V, S44D, S44G, S44N, S44R, V45A, V45M, T46A, T46I, N47D, N47K, N47R, S48L, S48P, S48T, V49A, V49M, K50E, K50G, K50R, K50T, G51E, T52A, T52M, N53K, N53S, A54V, and A54T.

11.-12. (canceled)

13. The composition according to any one of claims 1-4 and 7, wherein said variant sBCMA has at least 90% sequence identity to SEQ ID NO: 67.

14. The composition according to any one of claims 1-4 and 7, wherein said variant sBCMA has at least 90% sequence identity to SEQ ID NO: 68.

15. The composition according to any one of claims 1-4 and 7, wherein said variant sBCMA has at least 90% sequence identity to SEQ ID NO: 69.

16.-17. (canceled)

18. The composition according to any one of claims 1-4 and 7, wherein said variant sBCMA has the amino acid sequence of SEQ ID NO: 67.

19. The composition according to any one of claims 1-4 and 7, wherein said variant sBCMA has the amino acid sequence of SEQ ID NO: 68.

20. The composition according to any one of claims 1-4 and 7, wherein said variant sBCMA has the amino acid sequence of SEQ ID NO: 69.

21.-22. (canceled)

23. The composition according to any one of the preceding claims, wherein said variant sBCMA exhibits enhanced binding affinity for A Proliferation Inducing Ligand (APRIL) or B-cell Activating Factor of the TNF family (BAFF) as compared to SEQ ID NO:1.

24. The composition according to any one of claims 1-22, wherein said variant sBCMA exhibits enhanced binding affinity for APRIL and BAFF as compared to SEQ ID NO:1.

25. A nucleic acid encoding said variant sBCMA according to any one of the preceding claims.

26. An expression vector comprising said nucleic acid of claim 25.

27. A host cell comprising said nucleic acid of claim 25 or said expression vector of claim 26.

28. A method of making a variant sBCMA protein comprising: a) culturing said host cell of claim 27 under conditions wherein said Fc fusion protein is expressed;
and b) recovering said variant sBCMA protein.

29. A composition comprising an sBCMA variant—Fc fusion protein comprising:
a) a variant sBCMA domain comprising at least one amino acid substitution as compared to SEQ ID NO:1, wherein said amino acid substitution is at a position number selected from the group consisting of 1, 2, 3, 4, 5, 6, 7, 9, 10, 11, 12, 14, 16, 19, 20, 22, 23, 25, 26, 29, 31, 32, 35, 36, 38, 39, 42, 43, 44, 45, 46, 47, 48, 49, 50, 51, 52, 53, and 54, wherein the numbering is according to the EU index;
b) an optional linker; and
c) an Fc domain.

30.-78. (canceled)


SEQUENCE LISTING (88 sequences)

SEQ ID NO: 1 - protein, 54 aa, Artificial Sequence
  Extracellular domain of wild type human BCMA
MLQMAGQCSQNEYFDSLLHACIPCQLRCSSNTPPLTCQRYCNASVTNSVKGTNA

SEQ ID NO: 2 - protein, 54 aa, Artificial Sequence
  Variant BCMA
MSQMAGQCPQNKYFDSLLHACIPCQLRCSSDTPPLACQRYCSASVTNSVKGTSA

SEQ ID NO: 3 - protein, 54 aa, Artificial Sequence
  Variant BCMA
VLQMAGQCSQNEYFDSLLHACIPCQLRCSSNPPPLACQRYCNASVINSVKGTDV

SEQ ID NO: 4 - protein, 54 aa, Artificial Sequence
  Variant BCMA
MLRMAGQCSQNEYFDNLLHACIPCQLRCSSNTPPLACQRYCNTSVTNSVKGTNA

SEQ ID NO: 5 - protein, 54 aa, Artificial Sequence
  Variant BCMA
MLQMAGQCSQNEYLDGLLHACIPCQLRCSSNTPPLACQRYCNASATDSVKGTNA

SEQ ID NO: 6 - protein, 54 aa, Artificial Sequence
  Variant BCMA
TLQVAGQCFQNEYFDGLLHACIPCQLRCSSNAPPLTCRRYCNASVTNSVKGTNA

SEQ ID NO: 7 - protein, 54 aa, Artificial Sequence
  Variant BCMA
ALQMAGQCAQNEYFDSLLHACIPCQLRCSSNTPPLTCRRYCNASVTNSVKGTNA

SEQ ID NO: 8 - protein, 54 aa, Artificial Sequence
  Variant BCMA
MLQMAEQCSQNEYFDSLLHACIPCRLRCSSNTPPLTCRRYCNASVTNSVKGTNA

SEQ ID NO: 9 - protein, 54 aa, Artificial Sequence
  Variant BCMA
VLQIAEQCPQDEYFDSLLHACIPCQLRCSSNTPPLTCQRYCNASVTNSMKGMNV

SEQ ID NO: 10 - protein, 54 aa, Artificial Sequence
  Variant BCMA
MLQMAGQCSQDEYFDGLLHACIPCQLRCSSSTPPLTCQRYCNASVTNSVKGTNA

SEQ ID NO: 11 - protein, 54 aa, Artificial Sequence
  Variant BCMA
MLQMAGQCSQDEYFDSLLYACMPCQLRCSSNPPPLTCQRYCNASVTSSVKGTSA

SEQ ID NO: 12 - protein, 54 aa, Artificial Sequence
  Variant BCMA
MLQMAERCSQNEYFDSLLYACIPCQLRCSSNTPPSTCQRYCNASVTNSVKGTNA

SEQ ID NO: 13 - protein, 54 aa, Artificial Sequence
  Variant BCMA
MLQMAGQCSQNEYFDSLLYACIPCQLRCSSNTPPLTCQRYCDASVTNPVKGANA

SEQ ID NO: 14 - protein, 54 aa, Artificial Sequence
  Variant BCMA
VLQMAGQCSQNEYFDSLLHACIPCQLRCSSDIPPLACQRYCNASVTNSVKGTNA

SEQ ID NO: 15 - protein, 54 aa, Artificial Sequence
  Variant BCMA
VLQMTGQCSQNEYFDSLLLACIPCQLRCSSNTPPLACQRYCNASVTNSVKGTNA

SEQ ID NO: 16 - protein, 54 aa, Artificial Sequence
  Variant BCMA
TLQMAGQCSQNEYFDSLLHACIPCQLRCSSDAPPLACRRYCNADVTNSAEGTNA

SEQ ID NO: 17 - protein, 54 aa, Artificial Sequence
  Variant BCMA
VLQMAGQCSQNEYFDSLLHACIPCQLRCSSNTPPLACRRYCNVSVTNSVKGTNA

SEQ ID NO: 18 - protein, 54 aa, Artificial Sequence
  Variant BCMA
VSQMAGQCPHNEYFDSLLHACIPCQLRCSSNTPPLACRRYCNASVTNSVGGTNA

SEQ ID NO: 19 - protein, 54 aa, Artificial Sequence
  Variant BCMA
MLQMAGQCSQNEYFDSLLHACIPCQLRCSSNTPPLACRRYCNASVTNSVKGTSA

SEQ ID NO: 20 - protein, 54 aa, Artificial Sequence
  Variant BCMA
TSQMAGQCSQNEYFDSLLHACIPCQLRCSSNTPPPACRRYCNASVANSVRGTNA

SEQ ID NO: 21 - protein, 54 aa, Artificial Sequence
  Variant BCMA
MLQMTGQCSQNEYFDSLLHVCIPCQLRCSSNTPPLACRRYCNASVTNSVKGTNA

SEQ ID NO: 22 - protein, 54 aa, Artificial Sequence
  Variant BCMA
TLQMAGQCSQNEYFDGLLHACVPCQLRCSSNTPPLACQRYCNAGVANSAKGTNA

SEQ ID NO: 23 - protein, 54 aa, Artificial Sequence
  Variant BCMA
MLQMAGQCSQNEYFDGLLHACIPCQLRCSSNTPPLACQRYCNASVTNSVKGTNA

SEQ ID NO: 24 - protein, 54 aa, Artificial Sequence
  Variant BCMA
ILQMAGQCSQDEYFDGLLHACMPCQLRCASNTPPLACQRYCNAGVTNSVRGTNA

SEQ ID NO: 25 - protein, 54 aa, Artificial Sequence
  Variant BCMA
CCREAGQCSQDEYFDGLLHACIPCQLRCSSNTPPLPCQRYCNASVTNSVKGTNA

SEQ ID NO: 26 - protein, 54 aa, Artificial Sequence
  Variant BCMA
MLQMAGQCSQDEYFDSLLHACIPCQLRCSSDIPPLACQRYCNANVTDSVKGTDA

SEQ ID NO: 27 - protein, 54 aa, Artificial Sequence
  Variant BCMA
RCRMAGQCSQDEYFDSLLYACIPCQLRCSSNTPPLACQRYCSASATNSVKGTSA

SEQ ID NO: 28 - protein, 54 aa, Artificial Sequence
  Variant BCMA
MLQMAGQCSQNEYFDSLLYACIPCQLRCSSNTPPLACQRYCNAGVTNSVKGTNA

SEQ ID NO: 29 - protein, 54 aa, Artificial Sequence
  Variant BCMA
MLQMAGQCSQNEYFDSLLYACIPCQLRCSSNIPPLACQRYCNASVTNSAKGTNA

SEQ ID NO: 30 - protein, 54 aa, Artificial Sequence
  Variant BCMA
MLQMAGQCSQNEYFDSLLYACIPCQLRCSSSTPPLACQRYCNASATNSVKGTNA

SEQ ID NO: 31 - protein, 54 aa, Artificial Sequence
  Variant BCMA
MLQMAGQCSQNEYFDSLLYACIPCQLRCSSSTPPLACQRYCNASVTNSVKGTNA

SEQ ID NO: 32 - protein, 54 aa, Artificial Sequence
  Variant BCMA
MLQMAGQCSQNEYFDSLLYACIPCQLRCSSNTPPLPCQRYCNASVTNSVKGANA

SEQ ID NO: 33 - protein, 54 aa, Artificial Sequence
  Variant BCMA
MLQMAGQCSQNEYFDSLLYACIPCQLRCSSDTPPLTCQRYCNASVTNSVKGMNA

SEQ ID NO: 34 - protein, 54 aa, Artificial Sequence
  Variant BCMA
VLQMAGQCSQNEYFDSLLYACIPCQLRCSSNTPPLTCQRYCNASMTNSVKGTNA

SEQ ID NO: 35 - protein, 54 aa, Artificial Sequence
  Variant BCMA
MLQMAGQCSQNEYFDGLLYACIPCQLRCSSNTPPLTCQRYCNASVTDSVKGTNA

SEQ ID NO: 36 - protein, 54 aa, Artificial Sequence
  Variant BCMA
MLQMAGQCSQNEYFDGLLYACIPCQLRCSSNTPPLTCQRYCNASVTNSVTGTNA

SEQ ID NO: 37 - protein, 54 aa, Artificial Sequence
  Variant BCMA
MLQMAGQCSQNEYFDGLLYACIPCQLRCSSNTPPLTCQRYCNANVTNSVRGTNA

SEQ ID NO: 38 - protein, 54 aa, Artificial Sequence
  Variant BCMA
MLQMAGQCSQDEYFDSLLYACIPCQLRCSSNTPPLTCQRYCNASVTNTVKGTNA

SEQ ID NO: 39 - protein, 54 aa, Artificial Sequence
  Variant BCMA
MLQMAGQCPQDEYFDRLLHACIPCQLRCSSNAPPLTCRRYCNAGVINSVKGADT

SEQ ID NO: 40 - protein, 54 aa, Artificial Sequence
  Variant BCMA
MLQMAGQCSQDEYFDGLLHACIPCQLRCSSNTPPLTCQRYCNARVTNSVKGTNA

SEQ ID NO: 41 - protein, 54 aa, Artificial Sequence
  Variant BCMA
MLQMAGQCSQNEYFDSLLLACIPCQLRCSSNAPPLTCQRYCNAGVTNSVKEANA

SEQ ID NO: 42 - protein, 54 aa, Artificial Sequence
  Variant BCMA
MLQMAGQCSQNEYFDNLLYACIPCQLRCSSNTPPLACQRYCNASVTNSVRGTNA

SEQ ID NO: 43 - protein, 54 aa, Artificial Sequence
  Variant BCMA
VLQMAGQCSQNEYFDSLLYACIPCQLRCSSNTPPLACQHYCNASVANSVKGTNA

SEQ ID NO: 44 - protein, 54 aa, Artificial Sequence
  Variant BCMA
VLQMAGQCSQNEYFDSLLYACIPCQLRCSSNTPPLACQRYCNASVTNSVKGTNA

SEQ ID NO: 45 - protein, 54 aa, Artificial Sequence
  Variant BCMA
MLQMAGQCSQNEYFDSLLYACIPCQLRCSSNTPPLACQRYCDASVTSPVKGTNA

SEQ ID NO: 46 - protein, 54 aa, Artificial Sequence
  Variant BCMA
VLQMAGQCSQNEYFDSLLYACIPCQLRCSSNTPPLACQRYCNAGVTDSVKGTNA

SEQ ID NO: 47 - protein, 54 aa, Artificial Sequence
  Variant BCMA
VLQMAGQCSQNEYFDSLLYACIPCQLRCSSNTPPLACQRYCRASVTNSVKGTSA

SEQ ID NO: 48 - protein, 54 aa, Artificial Sequence
  Variant BCMA
MLQMAGQCSQNEYFDSLLYACIPCQLRCSSNTPPPACQRYCDASVINSAKGTNA

SEQ ID NO: 49 - protein, 54 aa, Artificial Sequence
  Variant BCMA
MLPMAGQCPQNEYFDSLLYACIPCQLRCSSSTPPLACQHYCNASVTRSVEGTNA

SEQ ID NO: 50 - protein, 54 aa, Artificial Sequence
  Variant BCMA
TLQMAGQCSQNEYFDSLLYACIPCQLRCSSNTPPLACQRYCNASVTNSVKGTNA

SEQ ID NO: 51 - protein, 54 aa, Artificial Sequence
  Variant BCMA
VLQMAGQCSQNEYFDNLLYACMPCQLRCSSNTPPLACQRYCNASVTNSVKGTNA

SEQ ID NO: 52 - protein, 54 aa, Artificial Sequence
  Variant BCMA
TLQMAGQCSQDEYFDSLLYACIPCQLRCSSNTPPLACQRYCSASATNSVKGTSA

SEQ ID NO: 53 - protein, 54 aa, Artificial Sequence
  Variant BCMA
MLQMAGQCSQDEYFDGLLYACIPCQLRCSSNTPPLACQRYCNASVTSSVKGTDA

SEQ ID NO: 54 - protein, 54 aa, Artificial Sequence
  Variant BCMA
VLQMAGQCPPNEYFDGLLYACIPCQFRCSSNTPPLACQRYCNVSVTNSVKGTDA

SEQ ID NO: 55 - protein, 54 aa, Artificial Sequence
  Variant BCMA
MLQMAGQCSQNEYFDGLLYACIPCQLRCSSNTPPLACQRYCNASVTNSAKGTDA

SEQ ID NO: 56 - protein, 54 aa, Artificial Sequence
  Variant BCMA
MLQMAGQCSQNEYFDGLLHACIPCQLRCSSNTPPLACQRYCNTGMTNSVKGTNA

SEQ ID NO: 57 - protein, 54 aa, Artificial Sequence
  Variant BCMA
MLQVAGQCPQNEYFDGLLHACIPCQLRCSSNTPPLACRRYCNASVTNSVKGTNA

SEQ ID NO: 58 - protein, 54 aa, Artificial Sequence
  Variant BCMA
MLQMAGQCPQSEYFDGLLHACIPCQLRCSSNTPPLACRRYCNASVTNSVKGTNA

SEQ ID NO: 59 - protein, 54 aa, Artificial Sequence
  Variant BCMA
MLQMAGQCSQDKYFDRLLHACIPCQLRCSSNTPPLACQRYCNASVTNSVKGMNA

SEQ ID NO: 60 - protein, 54 aa, Artificial Sequence
  Variant BCMA
MLQVAGQCSQNEYFDSLLHACIPCQLRCSSNIPPLACRRYCNTSATNPVKGTNA

SEQ ID NO: 61 - protein, 54 aa, Artificial Sequence
  Variant BCMA
MLQMAGQCPQDEYFDGLLHACIPCRLRCSSNTPPLTCQRYCNASVTNSVKGTNA

SEQ ID NO: 62 - protein, 54 aa, Artificial Sequence
  Variant BCMA
TLQMTGQCPQNEYFDGLLHACIPCRLRCSSDTPPLTCQRYCNASVTNSMKGTNA

SEQ ID NO: 63 - protein, 54 aa, Artificial Sequence
  Variant BCMA
MSQMAGQCPQNEYFDGLLHTCIPCQLRCSSNIPPLTCRRYCDASVANLVKGTNA

SEQ ID NO: 64 - protein, 54 aa, Artificial Sequence
  Variant BCMA
MLQMAGQCSQNEYFDGLLHACIPCRLRCSSNTPPLTCQRYCNASVANSVKGTNA

SEQ ID NO: 65 - protein, 54 aa, Artificial Sequence
  Variant BCMA
MLQMAEQCAQNEYFDGLLHACIPCRLRCSSDTPPLTCQRYCNASVTSSVKGMNA

SEQ ID NO: 66 - protein, 54 aa, Artificial Sequence
  Variant BCMA
MLQMAGQCSQNEYFDSLLYACIPCQLRCSSNTPPLTCRRYCNASVTNSVKGMNA

SEQ ID NO: 67 - protein, 54 aa, Artificial Sequence
  Variant BCMA
MLQMAGQCSQNEYFDGLLYACIPCQLRCSSNTPPLACQRYCNASVTNSVKGTDA

SEQ ID NO: 68 - protein, 54 aa, Artificial Sequence
  Variant BCMA
MLQMAGQCSQNEYFDGLLYACIPCQLRCSSNTPPLACQRYCNASVTNSVKGTNA

SEQ ID NO: 69 - protein, 54 aa, Artificial Sequence
  Variant BCMA
MLQMAGQCPQDEYFDGLLYACIPCQLRCSSNTPPLACQRYCNASVTSSVKGTDA

SEQ ID NO: 70 - protein, 54 aa, Artificial Sequence
  Variant BCMA
VSQTAGQCSQDEYFDSLLYACIPCQLRCSSNTPPLACQRYCNASVTNSVKGTNA

SEQ ID NO: 71 - protein, 54 aa, Artificial Sequence; identical to SEQ ID NO: 70
  Variant BCMA

SEQ ID NO: 72 - protein, 54 aa, Artificial Sequence
  Variant BCMA
VSQTAGQCSQNEYFDSLLYACIPCQLRCSSNTPPLICQRYCNASATNSMKGTNA

SEQ ID NO: 73 - protein, 54 aa, Artificial Sequence
  Variant BCMA
VSQTAGQCPRNEYFDSLLYACIPCQLRCSSNTPPLACQRYCNASVASSVKGTNA

SEQ ID NO: 74 - protein, 54 aa, Artificial Sequence
  Variant BCMA
VSQTAGQCSQNEYFDGLLHACIPCQLRCSSDIPPLACQRYCNASVTNSVKGTNA

SEQ ID NO: 75 - protein, 54 aa, Artificial Sequence
  Variant BCMA
VLQTAGQCSQNEYFDSLLHACIPCQLRCSSNTPPLACRRYCNASVTNSVKGTKA

SEQ ID NO: 76 - protein, 54 aa, Artificial Sequence; identical to SEQ ID NO: 16
  Variant BCMA

SEQ ID NO: 77 - protein, 54 aa, Artificial Sequence
  Variant BCMA
TLQMAGQCPQNEYFDSLLHACISCQLRCSSNTPPLACRRYCSASVTNPAKGTNV

SEQ ID NO: 78 - protein, 54 aa, Artificial Sequence
  Variant BCMA
MLQMAGQCSQNEYFDSLLYACIPCQLRCSSNTPPLACQRYCNASVTNSVKGTNA

SEQ ID NO: 79 - protein, 54 aa, Artificial Sequence
  Variant BCMA
MLQTAGQCSQNEYFDSLLHACIPCQLRCSSNTPPLACRRYCSAGVAKPVKGANA

SEQ ID NO: 80 - protein, 290 aa, Artificial Sequence
  sBCMA variant - Fc fusion protein
MLQMAGQCSQNEYFDGLLYACIPCQLRCSSNTPPLACQRYCNASVTNSVKGTDAGGGGSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 290 aa, Artificial Sequence
  sBCMA variant - Fc fusion protein
MLQMAGQCSQNEYFDGLLYACIPCQLRCSSNTPPLACQRYCNASVTNSVKGTNAGGGGSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 290 aa, Artificial Sequence
  sBCMA variant - Fc fusion protein
MLQMAGQCPQDEYFDGLLYACIPCQLRCSSNTPPLACQRYCNASVTSSVKGTDAGGGGSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 290 aa, Artificial Sequence
  sBCMA variant - Fc fusion protein
MLPMAGQCPQNEYFDSLLYACIPCQLRCSSSTPPLACQHYCNASVTRSVEGTNAGGGGSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 290 aa, Artificial Sequence
  sBCMA variant - Fc fusion protein
VSQTAGQCSQNEYFDGLLHACIPCQLRCSSDIPPLACQRYCNASVTNSVKGTNAGGGGSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 152 aa, Artificial Sequence [sequence omitted]

SEQ ID NO: 86 - protein, 146 aa, Artificial Sequence
  Extracellular domain of APRIL
AVLTQKQKKQHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 87 - protein, 6 aa, Artificial Sequence
  linker domain
IEGRMD

SEQ ID NO: 88 - protein, 5 aa, Artificial Sequence
  linker domain
GGGGS